(19) United States
(12) Patent Application Publication
(10) Pub. No.: US 2026/0078448 A1
(43) Pub. Date: Mar. 19, 2026

(54) METHODS OF DETECTING SJÖGREN'S SYNDROME USING SALIVARY EXOSOMES

(71) Applicants: Exosome Diagnostics, Inc., Waltham, MA (US); Trustees of Tufts College, Medford, MA (US)

(72) Inventors: Johan Karl Olov Skog, Lincoln, MA (US); Athena S. Papas, Boston, MA (US); Sudipto Kumar Chakrabortty, Waltham, MA (US); Wei Yu, Waltham, MA (US); Benjamin D. Sawicki, Medford, MA (US); Brian C. Haynes, Austin, TX (US); Shuran Xing, Cambridge, MA (US); Timothy Jeffrey Cole, Austin, TX (US); Christian Ray, Austin, TX (US)

(21) Appl. No.: 19/109,403

(22) PCT Filed: Sep. 7, 2023
(86) PCT No.: PCT/US2023/032192

§ 371 Date: Mar. 6, 2025

Related U.S. Application Data
(60) Provisional application No. 63/404,242, filed on Sep. 7, 2022.

(51) Int. Cl. C12Q 1/6883; A61K 45/06

(52) U.S. Cl. CPC C12Q 1/6883; A61K 45/06; C12Q 2600/158

(57) ABSTRACT

The present disclosure is directed to methods of using salivary exosomes to detect and treat Sjögren's syndrome in a subject.

### RELATED APPLICATIONS

This application claims priority to, and the benefit of, U.S. Provisional Application No. 63/404,242, filed Sep. 7, 2022, the contents of which are incorporated herein by reference in their entireties.

### BACKGROUND

Sjögren's syndrome (SS) is a systemic autoimmune disease in which inflammation progressively damages the moisture-producing glands such as the salivary glands and tear glands. An estimated four million Americans are thought to suffer from the disease with estimated 2.5 million undiagnosed, 90% of which are women with an average age of 40. The symptoms of SS overlap with other health conditions and co-morbidities, including other inflammatory diseases and disorders, other auto-immune diseases, allergies, drug side effects and menopause. Moreover, the diagnostic criteria have changed multiple times because autoantibodies are not present in all patients and salivary gland biopsy material can be difficult to obtain, and pathologists may not correctly apply standard diagnostic criteria, making SS difficult to diagnose. In fact, the average time to diagnosis is currently three years. While several biomarker diagnostic tests have been developed for Sjögren's syndrome, the gold standard remains the biopsy of salivary glands, which is an invasive, expensive, highly skill dependent, and time-consuming procedure with a potential side effect of permanent lip numbness. Thus, there is a need in the art for improved methods for diagnosing Sjögren's syndrome.

### SUMMARY

The present disclosure provides methods of determining if a subject is Sjögren's syndrome positive or is Sjögren's syndrome negative. In some embodiments, the method comprises a) determining the expression level of at least one biomarker selected from at least one biomarker signature in microvesicular RNA isolated from a saliva sample from the subject. In some embodiments the method comprises b) inputting the expression levels from step (a) into an algorithm to generate a score. In some embodiments the method comprises c) identifying if the subject is Sjögren's syndrome positive or is Sjögren's syndrome negative syndrome based on the score.

The present disclosure provides methods of identifying the risk of Sjögren's syndrome in a subject. In some embodiments the method comprises a) determining the expression level of at least one biomarker selected from at least one biomarker signature in microvesicular RNA isolated from a saliva sample from the subject. In some embodiments the method comprises b) inputting the expression levels from step (a) into an algorithm to generate a score. In some embodiments the method comprises c) identifying the risk of Sjögren's syndrome based on the score.

The present disclosure provides methods of treating Sjögren's syndrome in a subject. In some embodiments the method comprises a) determining the expression level of at least one biomarker selected from at least one biomarker signature in microvesicular RNA isolated from a saliva sample from the subject. In some embodiments, the method comprises b) inputting the expression levels from step (a) into an algorithm to generate a score. In some embodiments, the method comprises c) administering at least one treatment to the subject based on the score.

The present disclosure provides methods of distinguishing between SSA positive Sjögren's syndrome and SSA negative Sjögren's syndrome in a subject. In some embodiments, the method comprises determining the expression level of at least one biomarker selected from at least one biomarker signature in microvesicular RNA isolated from a saliva sample from the subject. In some embodiments the method comprises b) inputting the expression levels from step (a) into an algorithm to generate a score. In some embodiments the method comprises c) distinguishing between SSA positive Sjögren's syndrome and SSA negative Sjögren's syndrome in the subject based on the score.

In some embodiments of the preceding methods, the at least one biomarker signature is selected from: i) ISG15, RSAD2, TRIM38, and IFI6; ii) IFIH1, DDX60, OAS3 and ZC3HAV1; iii) RSAD2, IFI6, IFIT5 and CMPK2; iv) DDX60, OAS3, IFI6 and RSAD2; v) CMPK2, OAS1, OASL and ISG15; vi) ISG15, IFI16, RSAD2 and OAS1; vii) IFIH1, ISG15, EPSTI1 and IFI16; viii) SERPING1, RTP4, SLC4A11 and MRAS; ix) NT5C3A, IFIH1, RTP4 and IFI44L; and x) ISG15, IFIH1, IFI16 and SLC4A11.

In some embodiments of the preceding methods, the at least one biomarker signature is selected from: i) ANKRD29, PRRX2, OAS1, and MUC2; ii) ARSL, NKX6-2, HTRA3, and BSN; and iii) ZCCHC4, UGT2A1, IFIT1, and CD101-AS1.

In some embodiments of the preceding methods, step (a) comprises determining the expression level of at least two, or at least three of the biomarkers in the at least one biomarker signature. In some embodiments, step (a) comprises determining the expression level of each of the biomarkers in the at least one biomarker signature.

In some embodiments of the preceding methods, the algorithm is the product of a feature selection wrapper algorithm, a machine learning algorithm, a trained classifier built from at least one predictive classification algorithm or any combination thereof. In some embodiments, the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

In some embodiments of the proceeding methods, the algorithm is the product of a feature selection wrapper algorithm, machine learning algorithm, trained classifier, logistic regression model or any combination thereof, that was trained to identify Sjögren's syndrome in a subject. In some embodiments, the algorithm was trained using a) the expression levels of the at least one biomarker selected from the at least one biomarker signature in at least one biological sample from at least one subject who does not have Sjögren's syndrome. In some embodiments, the algorithm was trained using b) the expression levels of the at least one biomarker selected from the at least one biomarker signature in at least one biological sample from at least one subject who has Sjögren's syndrome. In some embodiments, the algorithm was trained using c) the expression levels of the at least one biomarker selected from the at least one biomarker signature in at least one biological sample from at least one subject who has SSA positive Sjögren's syndrome. In some embodiments the algorithm is trained using d) the expression levels of the at least one biomarker selected from the at least one biomarker signature in at least one biological sample from at least one subject who has SSA negative Sjögren's syndrome. In some embodiments the algorithm is trained using e) the expression levels of the at least one biomarker selected from the at least one biomarker signature in at least one biological sample from a subject who does not have Sjögren's syndrome and who does not exhibit sicca symptoms. In some embodiments the algorithm is trained using f) the expression levels of the at least one biomarker selected from the at least one biomarker signature in at least one biological sample from a subject who does not have Sjögren's syndrome but exhibits sicca symptoms. In some embodiments the algorithm is trained using g) the expression levels of the at least one biomarker selected from the at least one biomarker signature in at least one biological sample from a subject who has at least on alternative disease/disorder. In some embodiments the algorithm is trained using h) any combination thereof.

In some embodiments of the preceding methods, the saliva sample is collected using sample home-collection device.

In some embodiments of the preceding methods, prior to step (a), the method comprises i) isolating a plurality of microvesicles from the saliva sample from the subject. In some embodiments prior to step (a), the method comprises ii) extracting microvesicular RNA from the plurality of isolated microvesicles. In some embodiments, the method comprises i) prior to step (i), adding at least one stabilizing agent to the saliva sample, preferably wherein the at least one stabilizing agent is an RNAse inhibitor. In some embodiments, the method comprises ii) filtering the saliva samples, preferably filtering comprises using a filter with an average pore size of about 0.8 μm. In some embodiments, the method comprises iii) fragmenting the extracted microvesicular RNA. In some embodiments, the method comprises iv) contacting the extracted microvesicular RNA with Solid-phase reversible immobilization (SPRI) beads. In some embodiments the method comprises v) amplifying the extracted microvesicular RNA is using PCR, preferably wherein the amplification is performed for about 18 cycles.

In some embodiments of the preceding methods, the plurality of microvesicles is isolated from the saliva sample by contacting the saliva sample with at least one affinity agent that binds to at least one surface marker present on the surface the at least one microvesicle.

In some embodiments of the preceding methods, step (a) further comprises (i) determining the expression level of at least one reference biomarker. In some embodiments, step (a) further comprises (ii) normalizing the expression level of the at least one biomarker to the expression level of the at least one reference biomarker.

In some embodiments of the preceding methods, the expression levels from step (a) into an algorithm to generate a score comprises inputting the normalized expression levels from step (a) into an algorithm to generate a score.

In some embodiments of the preceding methods, determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof. In some embodiments, determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof, correspond to subject's transcriptome.

In some embodiments of the preceding methods, the method i) has a negative predictive value of at least about 80%, or at least about 85%, or at least about 90%, or at least about 95%, or at least about 99%, or at least about 99.9%. In some embodiments, the method ii) has a positive predictive value of at least about 80%, or at least about 85%, or at least about 90%, or at least about 95%, or at least about 99%, or at least about 99.9%. In some embodiments, the method iii) has a sensitivity of at least about 80%, or at least about 85%, or at least about 90%, or at least about 95%, or at least about 99%, or at least about 99.9%. In some embodiments, the method iv) has a specificity of at least about 80%, or at least about 85%, or at least about 90%, or at least about 95%, or at least about 99%, or at least about 99.9%. In some embodiments, the method comprises v) any combination thereof.

In some embodiments of the preceding methods, measuring expression levels in step (a) further comprises selectively enriching for the at least one biomarker.

In some embodiments of the preceding methods, the at least one biomarker is selectively enriched by hybrid-capture. Preferably, in some embodiments i) the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome. Preferably, in some embodiments ii) the hybrid-capture results in a significant depletion in microbial nucleic acids

In some embodiments of the preceding methods, the method further comprises administering at least one treatment to a subject identified as having Sjögren's syndrome.

In some embodiments of the preceding methods, the at least one treatment comprises i) administering at least one therapeutically effective amount of cevimeline, pilocarpine, a supersaturated calcium phosphate rinse, cyclosporine, tacrolimus eye drops, abatacept, rituximab, tocilizumab, hydroxypropyl cellulose, lifitegrast, LO2A eye drops, rebamipide eye drops, topical autologous serum, intravenous immunoglobulins, dexamethasone eye drops, an immunosuppressive medication, a nonsteroidal anti-inflammatory medication, an arthritis medication, an antifungal medication, hydroxychloroquine, methotrexate, LOU064, INCB050465 or any combination thereof. In some embodiments, the at least one treatment comprises ii) surgery, preferably wherein the surgery comprises sealing the tear ducts of the subject. In some embodiments, the at least one treatment comprises iii) administering at least one therapeutically effective amount of UCB5857, CFZ533, AMG557, IL-2, a combination of rituximab and belimumab, tocilizumab, abatacept, RSLV-132, VIB4920, iscalimab, baricitinib, nipocalimab, dazodalibep, MHV370, S95011, efgartigimod, tofacitinib, iguratomid, anifrolumab, branebrutinib, telitacicept, or any combination thereof. In some embodiments the treatment comprises iv) at least one AAV-based therapy, preferably wherein the at least one AAV-based therapy comprises an AAV-based vector comprising a nucleic acid sequence encoding at least one aquaporin protein, or a functional fragment thereof. In some embodiments, the treatment comprises iv) any combination thereof. Any of the above aspects can be combined with any other aspect described above or herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of graphs showing the mapping statistics (i.e. the percentage of final sequencing reads that are mapped to either intergenic nucleic acid, intronic nucleic acid, transcriptome nucleic acid, other genomic nucleic acid, or nucleic acid that is unmappable) obtained from saliva samples that were analyzed without the addition of an RNase inhibitor (left panel) and from saliva samples that were analyzed with the addition of an RNAse inhibitor.

FIG. 2 is a graph showing the mapping statistics of final sequencing reads obtained from saliva samples that were analyzed without filtering and from saliva samples that were analyzed with filtering.

FIG. 3 is a series of graphs showing genes detected (left panel) and the Gene 80 coverage (number of genes that have reads covering ≥80%; right panel) in the final sequencing analysis using varying fragmentation times to process the extracted microvesicular RNA.

FIG. 4 is a series of graphs showing genes detected (left panel) and the Gene 80 coverage (right panel) in the final sequencing analysis using various ratios of solid-phase reversible immobilization (SPRI) beads.

FIG. 5 is a graph showing the mapping statistics for libraries that were produced with a final PCR amplification of 19 cycles or 18 cycles.

FIG. 6 is a series of graphs showing the mapping statistics for the final sequencing reads obtained in the analysis of the Sjögren's syndrome saliva samples and various healthy matched control samples, as described in Example 1.

FIG. 7 is a series of graphs showing the biotype distribution for the final sequencing reads obtained in the analysis of the Sjögren's syndrome saliva samples and various healthy matched control saliva samples, as described in Example 1.

FIG. 8 is a series of graphs showing the number of genes detected (left panel) and the Gene 80 coverage (right panel) in the final sequencing analysis for the Sjögren's syndrome saliva samples, the healthy matched control saliva samples, the RA saliva samples and the SLE saliva samples, as described in Example 1.

FIG. 9 shows a heatmap of genes that are differentially expressed between healthy control saliva samples and Sjögren's syndrome saliva samples.

FIG. 10 shows a heat map of genes that are implicated in the interferon alpha and interferon beta response pathways that are differentially expressed between healthy control saliva samples and Sjögren's syndrome saliva samples. The genes are part of the Moserle INFA Response gene set (see Moserle L, Indraccolo S, Ghisi M, Frasson C, Fortunato E, Canevari S, Miotti S, Tosello V, Zamarchi R, Corradin A, Minuzzo S, Rossi E, Basso G, Amadori A. The side population of ovarian cancer cells is a primary target of IFN-alpha antitumor effects. Cancer Res. 2008 Jul. 15; 68(14):5658-68. doi: 10.1158/0008-5472.CAN-07-6341. PMID: 18632618, incorporated herein by reference in its entirety for all purposes).

FIG. 11 shows a heat map of genes that are implicated in the interferon alpha and interferon beta response pathways that are differentially expressed between healthy control saliva samples and Sjögren's syndrome saliva samples. The genes are part of the EINAV Interferon gene set (see Einav U, Tabach Y, Getz G, Yitzhaky A, Ozbek U, Amariglio N, Izraeli S, Rechavi G, Domany E. Gene expression analysis reveals a strong signature of an interferon-induced pathway in childhood lymphoblastic leukemia as well as in breast and ovarian cancer. Oncogene. 2005 Sep. 22; 24(42):6367-75. doi: 10.1038/sj.one.1208797. PMID: 16007187, incorporated herein by reference in its entirety for all purposes).

FIG. 12 shows a heat map of genes that are implicated in the interferon alpha and interferon beta response pathways that are differentially expressed between healthy control saliva samples and Sjögren's syndrome saliva samples. The genes are part of the Hecker INFB1 Targets gene set (see Hecker M, Hartmann C, Kandulski O, Paap B K, Koczan D, Thiesen H J, Zettl U K. Interferon-beta therapy in multiple sclerosis: the short-term and long-term effects on the patients' individual gene expression in peripheral blood. Mol Neurobiol. 2013 December; 48 (3): 737-56. doi: 10.1007/s12035-013-8463-1. Epub 2013 May 1. PMID: 23636981, incorporated herein by reference in its entirety for all purposes).

FIG. 13 is a graph showing the expression of various aquaporin genes in healthy control saliva samples (left box plots in each group) and Sjögren's syndrome saliva samples (right box plot in each group).

FIG. 14 shows the variable importance for various genes identified by the feature selection described in Example 1.

FIG. 15 shows receiver-operating characteristic (ROC) curve analysis for ISG15, RSAD2, TRIM38 and IFI6, as well as all four genes together as a single biomarker signature, for use in identifying the presence Sjögren's syndrome in a subject based on the expression of the genes in microvesicular RNA extracted from a saliva sample from the cohort 1 subject.

FIG. 16 shows ROC curve analysis for IFIH1, DDX60, OAS3 and ZC3HAV1, as well as all four genes together as a single biomarker signature, for use in identifying the presence Sjögren's syndrome in a subject based on the expression of the genes in microvesicular RNA extracted from a saliva sample from the cohort 1 subject.

FIG. 17 shows ROC curve analysis for RSAD2, IFI6, IFIT5 and CMPK2, as well as all four genes together as a single biomarker signature, for use in identifying the presence Sjögren's syndrome in a subject based on the expression of the genes in microvesicular RNA extracted from a saliva sample from the cohort 1 subject.

FIG. 18 shows ROC curve analysis for a biomarker signature comprising the biomarkers DDX60, OAS3, IFI6 and RSAD2, for use in identifying the presence Sjögren's syndrome in a subject based on the expression of the genes in microvesicular RNA extracted from a saliva sample from the cohort 1 subject.

FIG. 19 shows ROC curve analysis for ISG15, RSAD2, IFI16 and OAS1, as well as all four genes together as a single biomarker signature for use in identifying the presence Sjögren's syndrome in a subject based on the expression of the genes in microvesicular RNA extracted from a saliva sample from the cohort 1 subject.

FIG. 20 shows ROC curve analysis SERPING1, RTP4, SLC4A11 and MRAS, as well as all four genes together as a single biomarker signature for use in identifying the presence Sjögren's syndrome in a subject based on the expression of the genes in microvesicular RNA extracted from a saliva sample from the cohort 1 subject.

FIG. 21 shows ROC curve analysis for ISG15, IFIH1, EPSTI1 and IFI16, as well as all four genes together as a single biomarker signature for use in identifying the presence Sjögren's syndrome in a subject based on the expression of the genes in microvesicular RNA extracted from a saliva sample from the cohort 1 subject.

FIG. 22 shows ROC curve analysis NT5C3A, IFIH1, RTP4 and IFI44L, as well as all four genes together as a single biomarker signature for use in identifying the presence Sjögren's syndrome in a subject based on the expression of the genes in microvesicular RNA extracted from a saliva sample from the cohort 1 subject.

FIG. 23 shows ROC curve analysis ISG15, IFIH1, IFI16 and SLC4A11, as well as all four genes together as a single biomarker signature for use in identifying the presence Sjögren's syndrome in a subject based on the expression of the genes in microvesicular RNA extracted from a saliva sample from the cohort 1 subject.

FIG. 24A shows receiver-operating characteristic (ROC) curve analysis for a biomarker signature comprising the biomarkers ISG15, RSAD2, TRIM38 and IFI6, for use in identifying the presence Sjögren's syndrome in a subject based on the expression of the genes in microvesicular RNA extracted from a saliva sample from the cohort 2 subject. FIG. 24B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 24C shows a Confusion Matrix summarizing the performance of the gene signature on the subjects from cohort 2.

FIG. 25A shows ROC curve analysis for a biomarker signature comprising the biomarkers IFIH1, DDX60, OAS3 and ZC3HAV1, for use in identifying the presence Sjögren's syndrome in a subject based on the expression of the genes in microvesicular RNA extracted from a saliva sample from the cohort 2 subject. FIG. 25B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 25C shows a Confusion Matrix summarizing the performance of the gene signature on the subjects from cohort 2.

FIG. 26A shows ROC curve analysis for a biomarker signature comprising the biomarkers RSAD2, IFI6, IFIT5 and CMPK2, for use in identifying the presence Sjögren's syndrome in a subject based on the expression of the genes in microvesicular RNA extracted from a saliva sample from the cohort 2 subject. FIG. 26B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 26C shows a Confusion Matrix summarizing the performance of the gene signature on the subjects from cohort 2.

FIG. 27A shows ROC curve analysis for a biomarker signature comprising the biomarkers DDX60, OAS3, IFI6 and RSAD2, for use in identifying the presence Sjögren's syndrome in a subject based on the expression of the genes in microvesicular RNA extracted from a saliva sample from the cohort 2 subject. FIG. 27B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 27C shows a Confusion Matrix summarizing the performance of the gene signature on the subjects from cohort 2.

FIG. 28A shows ROC curve analysis for a biomarker signature comprising the biomarkers CMPK2, OAS1, OASL and ISG15, for use in identifying the presence Sjögren's syndrome in a subject based on the expression of the genes in microvesicular RNA extracted from a saliva sample from the cohort 2 subject. FIG. 28B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 28C shows a Confusion Matrix summarizing the performance of the gene signature on the subjects from cohort 2.

FIG. 29A shows ROC curve analysis for a biomarker signature comprising the biomarkers ISG15, RSAD2, IFI16 and OAS1, for use in identifying the presence Sjögren's syndrome in a subject based on the expression of the genes in microvesicular RNA extracted from a saliva sample from the cohort 2 subject. FIG. 29B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 29C shows a Confusion Matrix summarizing the performance of the gene signature on the subjects from cohort 2.

FIG. 30A shows ROC curve analysis for a biomarker signature comprising the biomarkers ISG15, IFIH1, EPSTI1 and IFI16, for use in identifying the presence Sjögren's syndrome in a subject based on the expression of the genes in microvesicular RNA extracted from a saliva sample from the cohort 2 subject. FIG. 30B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 30C shows a Confusion Matrix summarizing the performance of the gene signature on the subjects from cohort 2.

FIG. 31A shows ROC curve analysis for a biomarker signature comprising the biomarkers ISG15, IFIH1, IFI16 and SLC4A11, for use in identifying the presence Sjögren's syndrome in a subject based on the expression of the genes in microvesicular RNA extracted from a saliva sample from the cohort 2 subject. FIG. 31B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 31C shows a Confusion Matrix summarizing the performance of the gene signature on the subjects from cohort 2.

FIG. 32 shows a heatmap summarizing the performance of various gene signatures used to train logistic regression models using cohort 1 data and tested using a pilot dataset and cohort 2 data (SSA positive Sjögren's syndrome vs healthy without sicca symptoms; SSA positive Sjögren's syndrome vs all healthy (with and without sicca symptoms)).

FIG. 33 shows a heatmap summarizing univariate analysis of Boruta selected biomarkers in all pairwise comparisons in cohort 2 (SSA positive Sjögren's syndrome subjects compared to healthy subjects without sicca symptoms, SSA positive Sjögren's syndrome subjects compared to healthy subjects with sicca symptoms, SSA negative Sjögren's syndrome subjects compared to healthy subjects without sicca symptoms, and SSA negative Sjögren's syndrome subjects compared to healthy subjects with sicca symptoms).

FIG. 34A shows the variable importance for various genes identified by the feature selection described in Example 3. FIG. 34B shows a heatmap summarizing univariate analysis of 28 Boruta selected genes. The 28 genes were selected from 56 genes that were differentially expressed between healthy control saliva samples (both with and without sicca symptoms) and SSA positive Sjögren's syndrome saliva samples.

FIG. 35A shows an ROC curve analysis for a biomarker signature comprising the biomarkers ANKRD29, PRRX2, OAS1, and MUC2 for use in identifying the presence SSA positive Sjögren's syndrome in a subject based on the expression of the genes in microvesicular RNA extracted from a saliva sample from the cohort 2 subject. FIG. 35B shows an ROC curve analysis for the biomarker signature for use in identifying the presence SSA positive Sjögren's syndrome in a subject based on the expression of the genes in microvesicular RNA extracted from a saliva sample from the subject from cohort 1. FIG. 35C shows a Principal Component Analysis (PCA) of the expression of the gene signature in sequencing analysis of microvesicular RNA isolated from salivary microvesicles from subjects with SSA positive Sjögren's syndrome and healthy subjects (with and without sicca symptoms) in cohort 2.

FIG. 36A shows the variable importance for various genes identified by the feature selection described in Example 3. FIG. 36B shows a heatmap summarizing univariate analysis of 5 Boruta selected genes. The 5 genes were selected from 11 genes that were differentially expressed between healthy control saliva samples (with and without sicca symptoms) and SSA negative Sjögren's syndrome saliva samples.

FIG. 37A shows an ROC curve analysis for a biomarker signature comprising the biomarkers ARSL, NKX6-2, HTRA3, and BSN for use in identifying the presence SSA negative Sjögren's syndrome in a subject based on the expression of the genes in microvesicular RNA extracted from a saliva sample from the subject from cohort 2. FIG. 37B shows a Principal Component Analysis (PCA) of the expression of the gene signature in sequencing analysis of microvesicular RNA isolated from salivary microvesicles from subjects with SSA negative Sjögren's syndrome and healthy subjects (with and without sicca symptoms) in cohort 2.

FIG. 38A shows the variable importance for various genes identified by the feature selection described in Example 3. FIG. 38B shows a heatmap summarizing univariate analysis of 16 Boruta selected genes. The 16 genes were selected from 32 genes that were differentially expressed between SSA positive Sjögren's syndrome saliva samples and SSA negative Sjögren's syndrome saliva samples.

FIG. 39A shows an ROC curve analysis for a biomarker signature comprising the biomarkers ZCCHC4, UGT2A1, IFIT1, CD101-AS1 for use in identifying the presence SSA negative Sjögren's syndrome and SSA positive Sjögren's syndrome in a subject based on the expression of the genes in microvesicular RNA extracted from a saliva sample from the subject from cohort 2. FIG. 39B shows a Principal Component Analysis (PCA) of the expression of the gene signature in sequencing analysis of microvesicular RNA isolated from salivary microvesicles from subjects with SSA negative Sjögren's syndrome and from salivary microvesicles from subjects with SSA positive Sjögren's syndrome in cohort 2. SSA negative Sjögren's syndrome was confirmed by lip biopsy.

### DETAILED DESCRIPTION

The present disclosure provides methods of identifying and treating Sjögren's syndrome in a subject in need thereof.

Sjögren's syndrome (SS) is a systemic autoimmune disease in which inflammation progressively damages the moisture producing glands such as the salivary glands and the tear glands. Symptoms can include dry, irritated and red eyes, dry mouth and difficulty swallowing. Four million Americans are estimated to be suffering from the disease, 90% of which are women with an average age of 40.

Overlapping symptoms with other health conditions and co-morbidities make SS particularly difficult to diagnose, with average time to diagnosis of 3 years. Currently, diagnosis of SS is performed by either: a) measuring levels of SS-A (Ro) protein in a biological sample from a subject (about 70% of subjects with SS test positive for SS-A (“SSA positive Sjögren's syndrome” or “SSA+SS”)); b) measuring levels of SS-B (La) protein in a biological sample from a subject (about 40% of subjects with SS test positive for SS-B); c) measuring levels of anti-nuclear antibody (ANA) in a biological sample from a subject (about 70% of subjects with SS test positive for ANA) but ANA is also a marker for other autoimmune diseases such as systemic lupus erythematosus; d) measuring levels of rheumatoid factor (RF) in a biological sample from a subject (about 60%-70% of subjects with SS test positive for RF, but RF is also a marker for other rheumatic diseases such as rheumatoid arthritis and systemic lupus erythematosus); or e) performing a salivary gland biopsy (typically from the lower lip) to confirm inflammatory cell infiltration of the minor salivary glands. Of all the current diagnostic methods, salivary gland biopsy is considered the “gold standard”. However, biopsy is an invasive, expensive, highly skill-dependent, and time-consuming procedure. Moreover, biopsy can could potentially lead to permanent lip numbness. Thus, there is a clear need in the art for a less-invasive, inexpensive and easy-to-administer diagnostic test for SS.

Further, despite the reliance on measurements of autoantibody levels, particularly antibodies to SSA, as a primary diagnostic criterion, standardization of autoantibody detection is a major challenge (see Veenbergen S, et al. J Transl Autoimmun. 2021 Dec. 27; 5:100138, incorporated herein by reference in its entirety for all purposes). In addition to being a key criterion for SS diagnosis, subjects who are positive for anti-SSA antibodies are also thought to be at a higher risk of developing lymphomas, neurological abnormalities, recurring prostatomegaly, more severe dysfunction of the exocrine glands, a more intense lymphocytic infiltration of the minor salivary glands, and longer disease duration (see Veenbergen et al. and Fan G, et al. 2021 Mar. 1; 27(2):50-55; Vilchez-Oya, F, et al. (2022). Frontiers in Immunology, 13, incorporated herein by reference in its entirety for all purposes). Therefore, there is a demonstrated need to accurately diagnose Sjögren's syndrome patients who are positive for anti-SSA antibodies (“SSA positive SS” or “SSA positive Sjögren's Syndrome”), not only for improving time to diagnosis, but also as a tool that may allow for early diagnosis of additional comorbidities.

Additionally, although the majority of subjects with Sjögren's Syndrome are positive for antibodies against SSA, a portion of subjects with SS are not (“SSA negative SS” or “SSA negative Sjögren's Syndrome”). Importantly, anti-Ro/SSA is remains the only autoantibody included in the American College of Rheumatology/European League Against Rheumatism (ACR/EULAR) classification criteria for diagnosing Sjögren's syndrome. As such, subjects with Sjögren's syndrome who are negative for antibodies against SSA may experience delays in diagnosis as the presence of anti-SSA antibodies is necessary for ACR/EULAR classification (see id.). Therefore, there is a clear need in the art to develop diagnostic tools which will be accurate, despite the heterogeneity in clinical phenotypes.

As described above, overlapping symptoms with other health conditions and co-morbidities make SS particularly difficult to diagnose. For example, symptoms of dry eyes and/or dry mouth, or sicca, are common in subjects with SS; however, such symptoms are not limited to SS (non-SS related sicca). As such there is a need to distinguish between Sjögren's syndrome and other causes of sicca so that subjects with sicca symptoms related to Sjögren's syndrome are able to receive the appropriate treatments.

Extracellular membrane vesicles called microvesicles are shed by eukaryotic and prokaryotic cells, or budded off from the plasma membrane, to the exterior of the cell. These extracellular membrane vesicles are heterogeneous in size with diameters ranging from about 10 nm to about 5000 nm. As used herein, the term “microvesicle” encompasses all extracellular membrane vesicles with diameters ranging from about 10 nm to about 5000 nm, including those with diameters <0.8 μm. These extracellular membrane vesicles can include, but are not limited to, microvesicles, microvesicle-like particles, prostasomes, dexosomes, texosomes, ectosomes, oncosomes, apoptotic bodies, retrovirus-like particles, and human endogenous retrovirus (HERV) particles. As used herein, the term “microvesicle” also encompasses small microvesicles (approximately 10 to 1000 nm, and more often 30 to 200 nm in diameter) that are released by exocytosis of intracellular multivesicular bodies. Such small microvesicles are also sometimes referred to in the art as exosomes. As such, the terms “exosomes”, “extracellular vesicles”, “extracellular membrane vesicles”, and “microvesicles” are used interchangeably herein.

Microvesicles are known to contain nucleic acids, including various DNA and RNA types such as mRNA (messenger RNA), miRNA (micro RNA), tRNA (transfer RNA), piRNA (piwi-interacting RNA), snRNA (small nuclear RNA), snoRNA (small nucleolar RNA), and rRNA (ribosomal RNA), various classes of long non-coding RNA, including long intergenic non-coding RNA (lincRNA) as well as proteins. Recent studies reveal that nucleic acids within microvesicles have a role as biomarkers. For example, WO 2009/100029 describes, among other things, the use of nucleic acids extracted from microvesicles in Glioblastoma multiforme (GBM, a particularly aggressive form of cancer) patient serum for medical diagnosis, prognosis and therapy evaluation. WO 2009/100029 also describes the use of nucleic acids extracted from microvesicles in human urine for the same purposes. The use of nucleic acids extracted from microvesicles is considered to potentially circumvent the need for biopsies, highlighting the enormous diagnostic potential of microvesicle biology (Skog et al. Nature Cell Biology, 2008, 10 (12): 1470-1476).

Microvesicles can be isolated from liquid biopsy samples from a subject, involving biofluids such as whole blood, serum, plasma, urine, saliva and cerebrospinal fluid (CSF). The nucleic acids contained within the microvesicles can subsequently be extracted. The extracted nucleic acids, e.g., microvesicular RNA (also referred to as exosomal RNA), can be further analyzed based on detection of a biomarker or a combination of biomarkers. The analysis can be used to generate a clinical assessment that diagnoses a subject with a disease, predicts the disease outcome of the subject, stratifies the subject within a larger population of subjects, predicts whether the subject will respond to a particular therapy, or determines if a subject is responding to an administered therapy.

Analysis of salivary exosomes has primarily focused on small RNAs and has been limited due to the large contribution of sequencing reads from the oral microbiome. In fact, previous studies that analyzed microvesicular RNA extracted from salivary microvesicles found that ˜60-95% of sequencing reads mapped to exogenous (i.e., microbial) genomes and transcriptomes. The methods of the present disclosure overcome these previous limitations and unexpectedly allows for the analysis of mRNAs and long intervening/intergenic noncoding RNAs (lincRNAs) in nucleic acids extracted from salivary microvesicles. For the purpose of the present disclosure microvesicular RNA, interchangeable with extracellular RNA (exRNA) or cell-free RNA, describes RNA species present outside of the cells in which they were transcribed. Carried within extracellular vesicles, lipoproteins, and protein complexes, exRNAs are protected from ubiquitous RNA-degrading enzymes. exRNAs may be found in the environment or, in multicellular organisms, within the tissues or biological fluids such as venous blood, saliva, breast milk, vaginal fluid, urine, semen, and menstrual blood.

### General Methods of the Present Disclosure

The present disclosure provides a method of identifying the presence or absence of Sjögren's syndrome in a subject, the method comprising analyzing the expression level of at least one biomarker selected from at least one biomarker signature in microvesicular RNA isolated from a saliva sample from the subject.

The present disclosure provides a method of identifying the risk of Sjögren's syndrome in a subject, the method comprising analyzing the expression level of at least one biomarker selected from at least one biomarker signature in microvesicular RNA isolated from a saliva sample from the subject.

The present disclosure provides a method of determining if a subject is Sjögren's syndrome positive or is Sjögren's syndrome negative, the method comprising analyzing the expression level of at least one biomarker selected from at least one biomarker signature in microvesicular RNA isolated from a saliva sample from the subject. In some aspects, a subject that is Sjögren's syndrome negative is a subject who does not have Sjögren's syndrome but has at least one alternative disease/disorder. In some aspects, the at least one alternative disease/disorder causes the subject to exhibit one or more symptoms that are also symptoms of Sjögren's syndrome. In some aspects, the at least one alternative disease/disorder is selected from rheumatoid arthritis (RA) and Systemic Lupus Erythematosus (SLE). In some aspects, a subject that is Sjögren's syndrome negative is a subject who does not have Sjögren's syndrome but has sicca symptoms, wherein sicca symptoms include, dry eyes, dry mouth and any combination thereof. In some aspects, a subject that is Sjögren's syndrome negative is a subject who does not have Sjögren's syndrome and does not have at least one alternative disease/disorder, but has sicca symptoms, wherein sicca symptoms include, dry eyes, dry mouth and any combination thereof. In some aspects, a subject that is Sjögren's syndrome negative is a subject who does not have Sjögren's syndrome, does not have at least one alternative disease/disorder, and does not have sicca symptoms.

The present disclosure provides a method of treating Sjögren's syndrome in a subject, the method comprising analyzing the expression level of at least one biomarker selected from at least one biomarker signature in microvesicular RNA isolated from a saliva sample from the subject and administering at least one treatment to a subject identified as having Sjögren's syndrome based on the analysis of the microvesicular RNA.

The present disclosure provides a method of monitoring a Sjögren's syndrome treatment in a subject that has been administered the Sjögren's syndrome treatment, the method comprising analyzing the expression level of at least one biomarker selected from at least one biomarker signature in microvesicular RNA isolated from a saliva sample from the subject and determining whether the patient is responding to the Sjögren's syndrome treatment based on the analysis of the microvesicular RNA.

The present disclosure provides a method of identifying the presence or absence of SSA positive Sjögren's syndrome in a subject, the method comprising analyzing the expression level of at least one biomarker selected from at least one biomarker signature in microvesicular RNA isolated from a saliva sample from the subject.

The present disclosure provides a method of identifying the presence or absence of SSA negative Sjögren's syndrome in a subject, the method comprising analyzing the expression level of at least one biomarker selected from at least one biomarker signature in microvesicular RNA isolated from a saliva sample from the subject.

The present disclosure provides a method of distinguishing between SSA positive Sjögren's syndrome and SSA negative Sjögren's syndrome in a subject, the method comprising analyzing the expression level of at least one biomarker selected from at least one biomarker signature in microvesicular RNA isolated from a saliva sample from the subject.

In each of the preceding methods, and any of the methods described herein, in addition to analyzing the expression level of the at least one biomarker in microvesicular RNA isolated from a saliva sample, the expression level of the at least one biomarker can be analyzed in both microvesicular RNA and cell-free DNA from a saliva sample from the subject. Thus, in a non-limiting example, the present disclosure provides a method of identifying the presence or absence of Sjögren's syndrome in a subject, the method comprising analyzing the expression level of at least one biomarker selected from at least one biomarker signature in microvesicular RNA and cell-free DNA (cfDNA) isolated from a saliva sample from the subject.

The present disclosure provides a method of identifying the presence or absence of Sjögren's syndrome in a subject, the method comprising: a) determining the expression level of at least one biomarker selected from at least one biomarker signature in microvesicular RNA isolated from a saliva sample from the subject; and b) identifying the presence or absence of Sjögren's syndrome based on the expression level of the at least one biomarker.

In some aspects, identifying the presence or absence of Sjögren's syndrome based on the expression level of the at least one biomarker selected from at least one biomarker signature can comprise comparing the one or more expression levels to corresponding predetermined cutoff value and determining the presence or absence of Sjögren's syndrome in the subject based on the relationship between the one or more expression levels and the corresponding predetermined cutoff values (e.g. greater than, less than, or equal to). For example, in step (b) of the preceding method, the presence of Sjögren's syndrome in a subject can be identified when the expression level of the at least one biomarker signature is greater than or equal to its corresponding predetermined cutoff value and the absence of Sjögren's syndrome in the subject can be identified when the expression level of the at least one biomarker in the signature is less than its corresponding predetermined cutoff value. Alternatively, the presence of Sjögren's syndrome in a subject can be identified when the expression level of the at least one biomarker signature is less than or equal to its corresponding predetermined cutoff value and the absence of Sjögren's syndrome in the subject can be identified when the expression level of the at least one biomarker in the signature is greater than its corresponding predetermined cutoff value.

The present disclosure provides a method of identifying the risk of Sjögren's syndrome in a subject, the method comprising: a) determining the expression level of at least one biomarker selected from at least one biomarker signature in microvesicular RNA isolated from a saliva sample from the subject; and b) identifying the risk of Sjögren's syndrome based on the expression level of the at least one biomarker.

In some aspects, identifying the risk of Sjögren's syndrome based on the expression level of the at least one biomarker selected from at least one biomarker signature can comprise comparing the one or more expression levels to corresponding predetermined cutoff value and determining the risk of Sjögren's syndrome in the subject based on the relationship between the one or more expression levels and the corresponding predetermined cutoff values (e.g. greater than, less than, or equal to). For example, in step (b) of the preceding method, the subject can be identified as being at high risk for Sjögren's syndrome when the expression level of the at least one biomarker is greater than or equal to its corresponding predetermined cutoff value and the subject can be identified as being at low risk for Sjögren's syndrome when the expression level of the at least one biomarker is less than its corresponding predetermined cutoff value. Alternatively, the subject can be identified as being at low risk for Sjögren's syndrome when the expression level of the at least one biomarker is greater than or equal to its corresponding predetermined cutoff value and the subject can be identified as being at high risk for Sjögren's syndrome when the expression level of the at least one biomarker is less than its corresponding predetermined cutoff value.

The present disclosure provides a method of determining if a subject is Sjögren's syndrome positive or is Sjögren's syndrome negative: a) determining the expression level of at least one biomarker selected from at least one biomarker signature in microvesicular RNA isolated from a saliva sample from the subject; and b) identifying that the subject is Sjögren's syndrome positive or Sjögren's syndrome negative based on the expression level of the at least one biomarker.

In some aspects, identifying that the subject is Sjögren's syndrome positive or Sjögren's syndrome negative based on the expression level of the at least one biomarker selected from at least one biomarker signature can comprise comparing the one or more expression levels to corresponding predetermined cutoff value and determining if the subject is Sjögren's syndrome positive or Sjögren's syndrome negative based on the relationship between the one or more expression levels and the corresponding predetermined cutoff values (e.g. greater than, less than, or equal to). For example, in step (b) of the preceding method, a subject can be identified as Sjögren's syndrome positive when the expression level of the at least one biomarker is greater than or equal to its corresponding predetermined cutoff value and the subject can be identified as Sjögren's syndrome negative when the expression level of the at least one biomarker is less than its corresponding predetermined cutoff value. Alternatively, a subject can be identified as Sjögren's syndrome negative when the expression level of the at least one biomarker is greater than or equal to its corresponding predetermined cutoff value and the subject can be identified as Sjögren's syndrome positive when the expression level of the at least one biomarker is less than its corresponding predetermined cutoff value.

The present disclosure provides a method of monitoring a Sjögren's syndrome treatment in a subject that has been administered the Sjögren's syndrome treatment, the method comprising: a) determining the expression level of at least one biomarker selected from at least one biomarker signature in microvesicular RNA isolated from a saliva sample from the subject; and b) determining whether the subject is responding to the Sjögren's syndrome treatment based on the expression level of the at least one biomarker.

In some aspects, determining whether the subject is responding to the Sjögren's syndrome treatment based on the expression level of the at least on biomarker selected from at least one biomarker signature can comprise comparing the one or more expression levels to predetermined cutoff values and determining if the subject is responding based on the relationship between the one or more expression levels and the corresponding predetermined cutoff values (e.g. greater than, less than, or equal to). For example, in step (b) of the preceding method, a subject can be identified as responding to the Sjögren's syndrome treatment when the expression level of the at least one biomarker is greater than or equal to its corresponding predetermined cutoff value and the subject can be identified as not responding to the Sjögren's syndrome treatment when the expression level of the at least one biomarker is less than its corresponding predetermined cutoff value. Alternatively, a subject can be identified as not responding to the Sjögren's syndrome treatment when the expression level of the at least one biomarker is greater than or equal to its corresponding predetermined cutoff value and the subject can be identified as responding to the Sjögren's syndrome treatment when the expression level of the at least one biomarker is less than its corresponding predetermined cutoff value

The present disclosure provides a method of treating Sjögren's syndrome in a subject, the method comprising: a) determining the expression level of at least one biomarker selected from at least one biomarker signature in microvesicular RNA isolated from a saliva sample from the subject; and b) administering at least one treatment to the subject based on the expression level of the at least one biomarker selected from at least one biomarker signature.

In some aspects, administering at least one treatment to the subject based on the expression level of the at least one biomarker selected from at least one biomarker signature can further comprise comparing the one or more expression levels to corresponding predetermined cutoff values and determining if the treatment is needed based on the relationship between the one or more expression levels and the corresponding predetermined cutoff values (e.g. greater than, less than, or equal to). For example, in step (b) of the preceding method, the subject can be administered the at least one treatment when the expression level of the at least one biomarker is greater than or equal to its corresponding predetermined cutoff value. Alternatively, the subject can be administered the at least one treatment when the expression level of the at least one biomarker is less than its corresponding predetermined cutoff value.

The present disclosure provides a method of identifying the presence or absence of SSA positive Sjögren's syndrome in a subject, the method comprising: a) determining the expression level of at least one biomarker selected from at least one biomarker signature in microvesicular RNA isolated from a saliva sample from the subject; and b) identifying the presence or absence of SSA positive Sjögren's syndrome based on the expression level of the at least one biomarker.

In some aspects, identifying the presence or absence of SSA positive Sjögren's syndrome based on the expression level of the at least one biomarker selected from at least one biomarker signature can comprise comparing the one or more expression levels to corresponding predetermined cutoff value and determining the presence or absence of SSA positive Sjögren's syndrome in the subject based on the relationship between the one or more expression levels and the corresponding predetermined cutoff values (e.g. greater than, less than, or equal to). For example, in step (b) of the preceding method, the presence of SSA positive Sjögren's syndrome can be identified in the subject when the expression level of the at least one biomarker is greater than or equal to its corresponding predetermined cutoff value and the absence of SSA positive Sjögren's syndrome can be identified in the subject when the expression level of the at least one biomarker is less than its corresponding predetermined cutoff value. Alternatively, the absence of SSA positive Sjögren's syndrome can be identified in the subject when the expression level of the at least one biomarker is greater than or equal to its corresponding predetermined cutoff value and the presence of SSA positive Sjögren's syndrome can be identified in the subject when the expression level of the at least one biomarker is less than its corresponding predetermined cutoff value.

The present disclosure provides a method of identifying the presence or absence of SSA negative Sjögren's syndrome in a subject, the method comprising: a) determining the expression level of at least one biomarker selected from at least one biomarker signature in microvesicular RNA isolated from a saliva sample from the subject; and b) identifying the presence or absence of SSA negative Sjögren's syndrome based on the expression level of the at least one biomarker.

In some aspects, identifying the presence or absence of SSA negative Sjögren's syndrome based on the expression level of the at least one biomarker selected from at least one biomarker signature can comprise comparing the one or more expression levels to corresponding predetermined cutoff value and determining the presence or absence of SSA negative Sjögren's syndrome in the subject based on the relationship between the one or more expression levels and the corresponding predetermined cutoff values (e.g. greater than, less than, or equal to). For example, in step (b) of the preceding method, the presence of SSA negative Sjögren's syndrome can be identified in the subject when the expression level of the at least one biomarker is greater than or equal to its corresponding predetermined cutoff value and the absence of SSA negative Sjögren's syndrome can be identified in the subject when the expression level of the at least one biomarker is less than its corresponding predetermined cutoff value. Alternatively, the absence of SSA negative Sjögren's syndrome can be identified in the subject when the expression level of the at least one biomarker is greater than or equal to its corresponding predetermined cutoff value and the presence of SSA negative Sjögren's syndrome can be identified in the subject when the expression level of the at least one biomarker is less than its corresponding predetermined cutoff value.

The present disclosure provides a method of distinguishing between SSA positive Sjögren's syndrome and SSA negative Sjögren's syndrome in a subject, the method comprising: a) determining the expression level of at least one biomarker selected from at least one biomarker signature in microvesicular RNA isolated from a saliva sample from the subject; and b) distinguishing between SSA positive Sjögren's syndrome and SSA negative Sjögren's syndrome based on the expression level of the at least one biomarker.

In some aspects, distinguishing between SSA positive Sjögren's syndrome and SSA negative Sjögren's syndrome based on the expression level of the at least one biomarker selected from at least one biomarker signature can comprise comparing the one or more expression levels to corresponding predetermined cutoff value; and distinguishing between SSA positive Sjögren's syndrome and SSA negative Sjögren's syndrome based on the relationship between the one or more expression levels and the corresponding predetermined cutoff values (e.g. greater than, less than, or equal to). For example, in step (b) of the preceding method, SSA positive Sjögren's syndrome can be identified when the expression level of the at least one biomarker is greater than or equal to its corresponding predetermined cutoff value and SSA negative Sjögren's syndrome can be identified when the expression level of the at least one biomarker is less than its corresponding predetermined cutoff value. Alternatively, SSA negative Sjögren's syndrome can be identified when the expression level of the at least one biomarker is greater than or equal to its corresponding predetermined cutoff value and SSA positive Sjögren's syndrome can be identified when the expression level of the at least one biomarker is less than its corresponding predetermined cutoff value.

The present disclosure provides a method of identifying the presence or absence of Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of Sjögren's syndrome based on the score.

In some aspects, identifying the presence or absence of Sjögren's syndrome based on the score can comprise comparing the score to a predetermined cutoff value and determining the presence or absence of Sjögren's syndrome in the subject based on the relationship between the score and the predetermined cutoff value (e.g. is the score greater than the predetermined cutoff value, less than the predetermined cutoff value, or equal to the predetermined cutoff value). For example, in step (b) of the preceding method, the presence of Sjögren's syndrome in a subject can be identified when the score is greater than or equal to the corresponding predetermined cutoff value and the absence of Sjögren's syndrome in the subject can be identified when the score is less than the predetermined cutoff value. Alternatively, the presence of Sjögren's syndrome in a subject can be identified when the score is less than or equal to the predetermined cutoff value and the absence of Sjögren's syndrome in the subject can be identified when the score is greater than its corresponding predetermined cutoff value.

The present disclosure provides a method of identifying the risk of Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the risk of Sjögren's syndrome based on the score.

In some aspects, identifying the risk of Sjögren's syndrome based on the score can comprise comparing the score to a predetermined cutoff value and determining the risk of Sjögren's syndrome in the subject based on the relationship between the score and the predetermined cutoff value (e.g. is the score greater than the predetermined cutoff value, less than the predetermined cutoff value, or equal to the predetermined cutoff value). For example, in step (b) of the preceding method, the subject can be identified as being at high risk for Sjögren's syndrome when the score is greater than or equal to the predetermined cutoff value and the subject can be identified as being at low risk for Sjögren's syndrome when the score is less than the predetermined cutoff value. Alternatively, the subject can be identified as being at low risk for Sjögren's syndrome when the score is greater than or equal to the predetermined cutoff value and the subject can be identified as being at high risk for Sjögren's syndrome when the score is less than the predetermined cutoff value.

The present disclosure provides a method of determining if a subject is Sjögren's syndrome positive or is Sjögren's syndrome negative, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying if the subject is Sjögren's syndrome positive or is Sjögren's syndrome negative syndrome based on the score.

In some aspects, identifying if the subject is Sjögren's syndrome positive or is Sjögren's syndrome negative based on the score can comprise comparing the score to a predetermined cutoff value and determining if the subject is Sjögren's syndrome positive or is Sjögren's syndrome negative based on the relationship between the score and the predetermined cutoff value (e.g. is the score greater than the predetermined cutoff value, less than the predetermined cutoff value, or equal to the predetermined cutoff value). For example, in step (b) of the preceding method, a subject can be identified as Sjögren's syndrome positive when the score is greater than or equal to the predetermined cutoff value and the subject can be identified as Sjögren's syndrome negative when the score is less than the predetermined cutoff value. Alternatively, a subject can be identified as Sjögren's syndrome negative when the score is greater than or equal to the predetermined cutoff value and the subject can be identified as Sjögren's syndrome positive when the score is less than the predetermined cutoff value.

The present disclosure provides a method of monitoring a Sjögren's syndrome treatment in a subject that has been administered the Sjögren's syndrome treatment, the method comprising: a) determining the expression level of at least one biomarker selected from at least one biomarker signature in microvesicular RNA isolated from a saliva sample from the subject; b) inputting the expression levels from step (a) into an algorithm to generate a score; c) determining whether the subject is responding to the Sjögren's syndrome treatment based on the score.

In some aspects, determining whether the subject is responding to the Sjögren's syndrome treatment based on the score can comprise comparing the score to a predetermined cutoff value and determining if the subject is responding based on the relationship between the score and the predetermined cutoff value (e.g. is the score greater than the predetermined cutoff value, less than the predetermined cutoff value, or equal to the predetermined cutoff value). For example, in step (b) of the preceding method, a subject can be identified as responding to the Sjögren's syndrome treatment when the score is greater than or equal to the predetermined cutoff value and the subject can be identified as not responding to the Sjögren's syndrome treatment when the score is less than the predetermined cutoff value. Alternatively, a subject can be identified as not responding to the Sjögren's syndrome treatment when the score is greater than or equal to the predetermined cutoff value and the subject can be identified as responding to the Sjögren's syndrome treatment when the score is less than the predetermined cutoff value.

The present disclosure provides a method of treating Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) administering at least one treatment to the subject based on the score.

In some aspects, administering at least one treatment to the subject based on the score can further comprise comparing the score to a predetermined cutoff value and determining if the treatment is needed based on the relationship between the score and the predetermined cutoff value (e.g. is the score greater than the predetermined cutoff value, less than the predetermined cutoff value, or equal to the predetermined cutoff value). For example, in step (b) of the preceding method, the subject can be administered the at least one treatment when the score is greater than or equal to the predetermined cutoff value. Alternatively, the subject can be administered the at least one treatment when the score is less than the predetermined cutoff value.

The present disclosure provides a method of identifying the presence or absence of SSA positive Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of SSA positive Sjögren's syndrome based on the score.

In some aspects, identifying the presence or absence of SSA positive Sjögren's syndrome based on the score can comprise comparing the score to a predetermined cutoff value and determining the presence or absence of SSA positive Sjögren's syndrome in the subject based on the relationship between the score and the predetermined cutoff value (e.g. is the score greater than the predetermined cutoff value, less than the predetermined cutoff value, or equal to the predetermined cutoff value). For example, in step (b) of the preceding method, the presence of SSA positive Sjögren's syndrome can be identified in the subject when the score is greater than or equal to the predetermined cutoff value and the absence of SSA positive Sjögren's syndrome can be identified in the subject when the score is less than the predetermined cutoff value. Alternatively, the absence of SSA positive Sjögren's syndrome can be identified in the subject when the score is greater than or equal to the predetermined cutoff value and the presence of SSA positive Sjögren's syndrome can be identified in the subject when the score is less than the predetermined cutoff value.

The present disclosure provides a method of identifying the presence or absence of SSA negative Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of SSA negative Sjögren's syndrome based on the score.

In some aspects, identifying the presence or absence of SSA negative Sjögren's syndrome based on the score can comprise comparing the score to a predetermined cutoff value and determining the presence or absence of SSA negative Sjögren's syndrome in the subject based on the relationship between the score and the predetermined cutoff value (e.g. is the score greater than the predetermined cutoff value, less than the predetermined cutoff value, or equal to the predetermined cutoff value). For example, in step (b) of the preceding method, the presence of SSA negative Sjögren's syndrome can be identified in the subject when the score is greater than or equal to the predetermined cutoff value and the absence of SSA negative Sjögren's syndrome can be identified in the subject when the score is less than the predetermined cutoff value. Alternatively, the absence of SSA negative Sjögren's syndrome can be identified in the subject when the score is greater than or equal to the predetermined cutoff value and the presence of SSA negative Sjögren's syndrome can be identified in the subject when the score is less than the predetermined cutoff value.

The present disclosure provides a method of distinguishing between SSA positive Sjögren's syndrome and SSA negative Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) distinguishing between SSA positive Sjögren's syndrome and SSA negative Sjögren's syndrome in the subject based on the score.

In some aspects, distinguishing between SSA positive Sjögren's syndrome and SSA negative Sjögren's syndrome in the subject based on the score can comprise comparing the score to a predetermined cutoff value and distinguishing between SSA positive Sjögren's syndrome and SSA negative Sjögren's syndrome in the subject based on the relationship between the score and the predetermined cutoff value (e.g. is the score greater than the predetermined cutoff value, less than the predetermined cutoff value, or equal to the predetermined cutoff value). For example, in step (b) of the preceding method, SSA positive Sjögren's syndrome can be identified when the score is greater than or equal to the predetermined cutoff value and SSA negative Sjögren's syndrome can be identified when the score is less than the predetermined cutoff value. Alternatively, SSA negative Sjögren's syndrome can be identified when the score is greater than or equal to the predetermined cutoff value and SSA positive Sjögren's syndrome can be identified when the score is less than the predetermined cutoff value.

The present disclosure provides a method of identifying if a subject has Rheumatoid Arthritis (RA) or Sjögren's syndrome, the method comprising: a) determining the expression level of at least one biomarker selected from at least one biomarker signature in microvesicular RNA isolated from a saliva sample from the subject; b) identifying the subject as having either RA or Sjögren's syndrome based on the expression level(s) measured in step (a). In some aspects, identifying if a subject has Rheumatoid Arthritis (RA) or Sjögren's syndrome based on the expression level of the at least one biomarker selected from at least one biomarker signature can comprise comparing the one or more expression levels to corresponding predetermined cutoff value; and identifying if a subject has Rheumatoid Arthritis (RA) or Sjögren's syndrome based on the relationship between the one or more expression levels and the corresponding predetermined cutoff values (e.g. greater than, less than, or equal to).

The present disclosure provides a method of identifying if a subject has Systemic Lupus Erythematosus (SLE) or Sjögren's syndrome, the method comprising: a) determining the expression level of at least one biomarker selected from at least one biomarker signature in microvesicular RNA isolated from a saliva sample from the subject; b) identifying the subject as having either SLE or Sjögren's syndrome based on the expression level(s) measured in step (a). In some aspects, identifying if a subject has SLE or Sjögren's syndrome based on the expression level of the at least one biomarker selected from at least one biomarker signature can comprise comparing the one or more expression levels to corresponding predetermined cutoff value; and identifying if a subject has SLE or Sjögren's syndrome based on the relationship between the one or more expression levels and the corresponding predetermined cutoff values (e.g. greater than, less than, or equal to).

The present disclosure provides a method of identifying if a subject has non-Sjögren's syndrome related sicca symptoms or Sjögren's syndrome, the method comprising: a) determining the expression level of at least one biomarker selected from at least one biomarker signature in microvesicular RNA isolated from a saliva sample from the subject; b) identifying the subject as having either non-Sjögren's syndrome related sicca symptoms or Sjögren's syndrome based on the expression level(s) measured in step (a). In some aspects, identifying if a subject has non-Sjögren's syndrome related sicca symptoms or Sjögren's syndrome based on the expression level of the at least one biomarker selected from at least one biomarker signature can comprise comparing the one or more expression levels to corresponding predetermined cutoff value; and identifying if a subject has non-Sjögren's syndrome related sicca symptoms or Sjögren's syndrome based on the relationship between the one or more expression levels and the corresponding predetermined cutoff values (e.g. greater than, less than, or equal to).

In some aspects of the proceeding methods the Sjögren's syndrome is SSA positive Sjögren's syndrome. In some aspects of the proceeding methods the Sjögren's syndrome is SSA negative Sjögren's syndrome.

The present disclosure provides a method of identifying if a subject has Rheumatoid Arthritis (RA) or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either RA or Sjögren's syndrome based on the score. In some aspects, identifying if a subject has Rheumatoid Arthritis (RA) or Sjögren's syndrome based on the score can comprise comparing the score to a predetermined cutoff value; and identifying if a subject has Rheumatoid Arthritis (RA) or Sjögren's syndrome based on the relationship between the score and the predetermined cutoff value (e.g. greater than, less than, or equal to).

The present disclosure provides a method of identifying if a subject has Systemic Lupus Erythematosus (SLE) or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either SLE or Sjögren's syndrome based on the score. In some aspects, identifying if a subject has SLE or Sjögren's syndrome based on the score can comprise comparing the score to a predetermined cutoff value; and identifying if a subject has SLE or Sjögren's syndrome based on the relationship between the score and the predetermined cutoff value (e.g., greater than, less than, or equal to).

The present disclosure provides a method of identifying if a subject has non-Sjögren's syndrome related sicca symptoms or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either non-Sjögren's syndrome related sicca symptoms or Sjögren's syndrome based on the score. In some aspects, identifying if a subject has non-Sjögren's syndrome related sicca symptoms or Sjögren's syndrome based on the score can comprise comparing the score to a predetermined cutoff value; and identifying if a subject has non-Sjögren's syndrome related sicca symptoms or Sjögren's syndrome based on the relationship between the score and the predetermined cutoff value (e.g. greater than, less than, or equal to).

In some aspects of the proceeding methods the Sjögren's syndrome is SSA positive Sjögren's syndrome. In some aspects of the proceeding methods the Sjögren's syndrome is SSA negative Sjögren's syndrome.

The present disclosure provides a method of identifying the presence or absence of Sjögren's syndrome in a subject, the method comprising: a) determining the expression level of at least one upregulated biomarker and the expression level of at least one downregulated biomarker in microvesicular RNA isolated from a saliva sample from the subject; b) comparing the expression level of each of the biomarkers to a corresponding predetermined cutoff value for each biomarker; and c) identifying the presence of Sjögren's syndrome in the subject when the expression level of the at least one upregulated biomarker is greater than or equal to its corresponding predetermined cutoff value and the expression level of the at least one downregulated biomarker is less than or equal to its corresponding predetermined cutoff value or identifying the absence of Sjögren's syndrome in the subject when the expression level of the at least one upregulated biomarker is less than its corresponding predetermined cutoff value and the expression level of the at least one downregulated biomarker is greater than its corresponding predetermined cutoff value. In some aspects, the Sjögren's syndrome is SSA positive Sjögren's syndrome. In some aspects, the Sjögren's syndrome is SSA negative Sjögren's syndrome.

The present disclosure provides a method of treating Sjögren's syndrome in a subject, the method comprising: a) determining the expression level of at least one upregulated biomarker and the expression level of at least one downregulated biomarker in microvesicular RNA isolated from a saliva sample from the subject; b) comparing the expression level of each of the biomarkers to a corresponding predetermined cutoff value for each biomarker; and c) administering at least one treatment to the subject when the expression level of the at least one upregulated biomarker is greater than or equal to its corresponding predetermined cutoff value and the expression level of the at least one downregulated biomarker is less than or equal to its corresponding predetermined cutoff value. In some aspects, the Sjögren's syndrome is SSA positive Sjögren's syndrome. In some aspects, the Sjögren's syndrome is SSA negative Sjögren's syndrome.

The present disclosure provides a method of identifying the presence or absence of Sjögren's syndrome in a subject, the method comprising: a) determining the expression level of at least one upregulated biomarker in microvesicular RNA isolated from a saliva sample from the subject; b) comparing the expression level of each of the biomarkers to a corresponding predetermined cutoff value for each biomarker; and c) identifying the presence of Sjögren's syndrome in the subject when the expression level of the at least one upregulated biomarker is greater than or equal to its corresponding predetermined cutoff value or identifying the absence of Sjögren's syndrome in the subject when the expression level of the at least one upregulated biomarker is less than its corresponding predetermined cutoff value. In some aspects, the Sjögren's syndrome is SSA positive Sjögren's syndrome. In some aspects, the Sjögren's syndrome is SSA negative Sjögren's syndrome.

The present disclosure provides a method of treating Sjögren's syndrome in a subject, the method comprising: a) determining the expression level of at least one upregulated biomarker in microvesicular RNA isolated from a saliva sample from the subject; b) comparing the expression level of each of the biomarkers to a corresponding predetermined cutoff value for each biomarker; and c) administering at least one treatment to the subject when the expression level of the at least one upregulated biomarker is greater than or equal to its corresponding predetermined cutoff value. In some aspects, the Sjögren's syndrome is SSA positive Sjögren's syndrome. In some aspects, the Sjögren's syndrome is SSA negative Sjögren's syndrome.

The present disclosure provides a method of identifying the presence or absence of Sjögren's syndrome in a subject, the method comprising: a) determining the expression level of at least one downregulated biomarker in microvesicular RNA isolated from a saliva sample from the subject; b) comparing the expression level of each of the biomarkers to a corresponding predetermined cutoff value for each biomarker; and c) identifying the presence of Sjögren's syndrome in the subject when the expression level of the at least one downregulated biomarker is less than or equal to its corresponding predetermined cutoff value or identifying the absence of Sjögren's syndrome in the subject when the expression level of the at least one downregulated biomarker is greater than its corresponding predetermined cutoff value.

The present disclosure provides a method of treating Sjögren's syndrome in a subject, the method comprising: a) determining the expression level of at least one downregulated biomarker in microvesicular RNA isolated from a saliva sample from the subject; b) comparing the expression level of each of the biomarkers to a corresponding predetermined cutoff value for each biomarker; and c) administering at least one treatment to the subject when the expression level of the at least one downregulated biomarker is less than or equal to its corresponding predetermined cutoff value. In some aspects, the Sjögren's syndrome is SSA positive Sjögren's syndrome. In some aspects, the Sjögren's syndrome is SSA negative Sjögren's syndrome.

### Biomarker Signatures of the Present Disclosure

In some aspects, a biomarker signature comprises, consists essentially of, or consists of the biomarkers: RSAD2, OAS3, OAS2, IFIT1, OAS1, IFIT5, ISG15, IFIT3, IFI6, DDX60, OASL, USP18, GRAMD1B, LY6E, TRIM38, IFI44L, SLC4A11, PML, MX1, EPSTI1, IFIH1, EIF2AK2, XAF1, IFIT2, TRIM22, RFLNB, RTP4, KPTN, IFITM1, TMEM123, LINC01473, OTOF, GPRC5C, ISY1-RAB43, ZBP1, DDX58, IFITM3, NT5C3A, CMPK2, TBC1D16, IFI16, SHISA5, SERPING1, SP100, HERC5, BATF2, SHC2, UBE2L6, GLIS2, ZC3HAV1, GRAMD1A, TNFSF10, APOBEC3F, SNHG15, PDCD4-AS1, TOX, VAMP5, ERICH1, MRAS, SAMHD1 and TP5313.

In some aspects, a biomarker signature comprises, consists essentially of, or consists of the biomarkers: DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22, RFLNB and PML.

In some aspects, a biomarker signature comprises, consists essentially of, or consists of the biomarkers: DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22 and PML.

In some aspects, a biomarker signature comprises, consists essentially of, or consists of the biomarkers: DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5, BATF2 and DEFA1.

In some aspects, a biomarker signatures comprises, consists essentially of, or consists of the biomarkers: DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5 and BATF2.

In some aspects, a biomarker signature comprises, consists essentially of, or consists of the biomarkers: ISG15, RSAD2, TRIM38, and IFI6.

In some aspects, a biomarker signature comprises, consists essentially of, or consists of the biomarkers: TP5313, NT5C3A, SAMHD1, IFITM3, XAF1, GRAMD1A, SHC2, TBC1D16, ERICH1, OTOF, APOBEC3F, SP100, GLIS2, RTP4, SERPING1, TMEM123, EIF2AK2, HERC5, LINC01473, KPTN, IFITM1, IFIT2, DDX58, SHISA5, IFI44L, IFIT1, TNFSF10, UBE2L6, USP18, BATF2, VAMP5, OAS2, GPRC5C, ZBP1, SNHG15, TOX, LY6E, IFIT3, RFLNB, MX1, PML, TRIM22, IFI16, SLC4A11, EPSTI1, MRAS, ISG15, GRAMD1B, OAS1, OASL, TRIM38, IFI6, IFIT5, RSAD2, CMPK2, ZC3HAV1, IFIH1, OAS3 and DDX60.

In some aspects, a biomarker signature comprises, consists essentially of, or consists of the biomarkers: IFIH1, DDX60, OAS3 and ZC3HAV1.

In some aspects, a biomarker signature comprises, consists essentially of, or consists of the biomarkers: RSAD2, IFI6, IFIT5 and CMPK2.

In some aspects, a biomarker signature comprises, consists essentially of, or consists of the biomarkers: DDX60, OAS3, IFI6 and RSAD2.

In some aspects, a biomarker signature comprises, consists essentially of, or consists of the biomarkers: CMPK2, OAS1, OASL and ISG15.

In some aspect, a biomarker signature comprises, consists essentially of, or consists of the biomarkers: IFIH1, ISG15, EPSTI1, IFI16, RSAD2 and OAS1.

In some aspect, a biomarker signature comprises, consists essentially of, or consists of the biomarkers: ISG15, IFI16, RSAD2 and OAS1.

In some aspects, a biomarker signature comprises, consists essentially of, or consists of the biomarkers: IFIH1, ISG15, EPSTI1 and IFI16.

In some aspects, a biomarker signature comprises, consists essentially of, or consists of the biomarkers: ISG15, RSAD2, IFI16 and OAS1.

In some aspects, a biomarker signature comprises, consists essentially of, or consists of the biomarkers: SERPING1, RTP4, SLC4A11 and MRAS.

In some aspects, a biomarker signature comprises, consists essentially of, or consists of the biomarkers: ISG15, IFIH1, EPSTI1 and IFI16.

In some aspects, a biomarker signature comprises, consists essentially of, or consists of the biomarkers: NT5C3A, IFIH1, RTP4 and IFI44L.

In some aspects, a biomarker signature comprises, consists essentially of, or consists of the biomarkers: ISG15, IFIH1, IFI16 and SLC4A11.

In some aspects, a biomarker signature comprises, consists essentially of, or consists of the biomarkers: AQP1, AQP3, AQP4, AQP4-AS1, AQP5 and AQP7.

In some aspects, a biomarker signature comprises, consists essentially of, or consists of the biomarkers: AQP9.

In some aspects, a biomarker signature comprises, consists essentially of, or consists of the biomarkers: AQP9 and AQP1.

In some aspects, a biomarker signature is a biomarker signature related to response to interferons.

In some aspects, a biomarker signature related to response to interferons comprises, consists essentially of, or consists of the biomarkers: ISG15, IFI6, RXRA, IFIT1, STAT1, APOBEC3G, SP110, ERG, MORC3, IFI44L, MX1, SP100, LY6E, IFI44, ADAR, OAS1, IRF9, IFIT3, EIF2AK2, TGIF1, BST2, OAS2, CMTR1, UBE2L6, BRD3, IFI35 and IFI30.

In some aspects, a biomarker signature related to response to interferons is a biomarker signature related to response to interferon alpha. In some aspects, a biomarker signature related to response to interferon alpha comprises, consists essentially of, or consists of the biomarkers: IFIH1, MX1, GBP1, TNFSF10, OAS1, IFIT1, IFIT5, IFI44L, CXCL10, IFIT3, OAS2, OASL, IFI16, STAT1, ZC3HAV1, TRIM22, RSAD2, IFITM1, IFI44, IFIT2, DDX58, DDX60, USP18, RTP4, SAMD9, HERC5, SAMD9L, CMPK2, CD274, EPSTI1 and DDX60L.

In some aspects, a biomarker signature related to response to interferons is a biomarker signature related to response to interferon beta. In some aspects, a biomarker signature related to response to interferon alpha comprises, consists essentially of, or consists of the biomarkers: PDZK1IP1, MYL9, SPON2, IFI44, IFI44L, LILRA3, CHI3L1, CMTM5, CLU, CMPK2, CMTM2, DTX3L, TYMP, EGR1, EGR2, APOBEC3A, SAMD9L, FCER1A, DDX58, IFIT5, IFI6, STAP1, GBP1, GGTA1, LAMP3, GP9, TRBV27, FFAR2, GZMB, TREML1, IFI27, IFI35, IFIT2, IFIT1, IFIT3, CXCL8, CXCL10, IRF7, ITGA2B, JUP, KCNJ15, KLRD1, ARG1, IFITM3P7, LGALS3BP, CYP4F3, LY6E, MMP9, MX1, MX2, OAS1, OAS2, OAS3, GOS2, LAP3, HERC5, MS4A4A, PLSCR1, XAF1, SAMD9, HERC6, TMEM140, DDX60, EIF2AK2, PROS1, CABP5, HES4, RPS23, CCL2, CCL8, RTP4, MMP25, PARP12, SIGLEC1, STAT1, SERPING1, C1QA, C1QB, TNFAIP6, C1QC, C3AR1, DHX58, ZBP1, TCL1A, PARP9, SH3BGRL2, OASL, PNPT1, MGAM, RSAD2, CD163, EPSTI1, APOBEC3B, ISG15 and SCO2.

In some aspects, a biomarker signature comprises, consists essentially of, or consists of the biomarkers: C19orf48, RNF26, NOS2, ZNF775, ANKRD29, OAS1, ARSL, LAMB1, and TUBB3, ARSL, CTSC, ZCCHC4, UGT2A1, IFIT1, CD101-AS1, ANKRD29, PRRX2, OAS1, MUC2, ARSL, NKX6-2, HTRA3, BSN, ZCCHC4, UGT2A1, IFIT1, and CD101-AS1.

In some aspects, a biomarker signature comprises, consists essentially of, or consists of the biomarkers: C19orf48, HNRNPA2B1, RNF26, ZNF542P, CHRFAM7A, ENSG00000285818, CENPO, POLR1G, RASL11A, RPRM, DDR2, SSPN, ACP2, ZNF688, PLEK2, CHST13, SERPINF2, NOS2, EFCAB12, LAMB1, FGF7P7, KLC4, ABCA13, RTKN2, TPPP, and BPIFB4.

In some aspects, a biomarker signature comprises, consists essentially of, or consists of the biomarkers: ZNF775, OAS1, TAF6L, MUC2, SLC17A9, OAS2, THBS1, ENSG00000260989, NOS2, EPSTI1, KPNB1, NRSN2, MX1, RSAD2, PARP9, S100A7, IFIT1, DPYSL4, ISG15, ANKRD29, PRRX2, TXLNB, RAD9B, ENSG00000276490, TNFRSF25 and SCAND2P.

In some aspects, a biomarker signature comprises, consists essentially of, or consists of the biomarkers: AP4E1, ARSL, TUBB3, CBARP, CRACDL, LAMB1, LHPP, ZNF846 and SLC35F3.

In some aspects, a biomarker signature comprises, consists essentially of, or consists of the biomarkers: ARSL and CTSC.

In some aspects, a biomarker signature comprises, consists essentially of, or consists of the biomarkers: DCUNID2, KCNC3, NRP2, IKZF4, OLFML2A, CNTN4-AS1, CCDC177, CARNS1, OR6B3, CEACAM22P, HECTD3, ENSG00000227678, NPR3, NOS3, SPAG5-AS1, FBF1, TRIM22, IFI44L, IFIT2, THBD, KPNB1, DOX60, ENSG00000259732, GBP5, OASL, SIX1, MX1, SCNM1, ENSG00000259345, OAS3, KLK14, LY6E, S100A7, IFIT3, IFI6, FMO2, RSAD2, NOS2, SPRR2F, IFIT1, ISG15, PARP9, ANOS2P, THBS1, TNFRSF25, HERC5, MUC2, TXLNB, OAS1, CHRFAM7A, EPSTI1, SLC17A9, OAS2, PRRX2, ANKRD29, and ENSG00000260989.

In some aspects, a biomarker signature comprises, consists essentially of, or consists of the biomarkers: OAS1, MUC2, NOS2, ENSG00000260989, OAS2, SLC17A9, SLC17A9, CHRFAM7A, EPSTI1, LY6E, THBS1, RSAD2, PARP9, IFI6, S100A7, OAS3, SPRR2F, IFIT1, HERC5, IFIT3, ANKRD29, ANOS2P, ISG15, TXLNB, PRRX2, TNFRSF25, FMO2, ENSG00000259345, and KLK14.

In some aspects, a biomarker signature comprises, consists essentially of, or consists of the biomarkers: SMAD4, ENSG00000279159, MAP3K15, DLC1, CB84, NOX4, CTSC, BSN, HTRA3, NKX6-2, and ARSL.

In some aspects, a biomarker signature comprises, consists essentially of, or consists of the biomarkers: ARSL, NKX6-2, CTSC, BSN and HTRA3.

In some aspects, a biomarker signature comprises, consists essentially of, or consists of the biomarkers: RNF157-AS1, MYO3A, LY6E, HECTD3, CDCA2, TRIM22, CCL17, DDX58, OTOF, RTP4, IGSF9, DDX60L, ISG15, RSAD2, RNF213, OASL, SIGLEC1, IFI44, IFIT2, OAS2, OAS3, IFI4L, IFIT5, IFIT3, CD101-AS1, OAS1, FAM111A-DT, IFIT1, MX1, HERC5, UGT2A1, and ZCCHC4.

In some aspects, a biomarker signature comprises, consists essentially of, or consists of the biomarkers: ZCCHC4, OAS1, IFIT5, IFI44L, MX1, HERC5, OAS2, IFIT3, IFIT1, FAM111A-DT, SIGLEC1, IFIT2, OAS3, IFI44, CD101-AS1, UGT2A1.

In some aspects, a biomarker signature comprises, consists essentially of, or consists of the biomarkers: C19orf48, RNF26, and NOS2.

In some aspects, a biomarker signature comprises, consists essentially of, or consists of the biomarkers: ZNF775, ANKRD29, and OAS1.

In some aspects, a biomarker signature comprises, consists essentially of, or consists of the biomarkers: ARSL, LAMB1, and TUBB3.

In some aspects, a biomarker signature comprises, consists essentially of, or consists of the biomarkers: ARSL and CTSC.

In some aspects, a biomarker signature comprises, consists essentially of, or consists of the biomarkers: ZCCHC4, UGT2A1, IFIT1, and CD101-AS1.

In some aspects, a biomarker signature comprises, consists essentially of, or consists of the biomarkers: ANKRD29, PRRX2, OAS1, and MUC2.

In some aspects, a biomarker signature comprises, consists essentially of, or consists of the biomarkers: ARSL, NKX6-2, HTRA3, and BSN.

In some aspects of the preceding methods, step (a) can comprise determining the expression level of at least two, or at least three, or at least four, or at least five, or at least six, or at least seven, or at least eight, or at least nine, or at least 10, or at least 11, or at least 12, or at least 13, or at least four of the 14 biomarkers, or at least 15, or at least 16, or at least 17, or at least 18, or at least 19, or at least 20, or at least 21, or at least 22, or at least 23, or at least 24, or at least 25, or at least 26, or at least 27, or at least 28, or at least 29, or at least 30, or at least 31, or at least 32, or at least 33, or at least 34, or at least 35, or at least 36, or at least 37, or at least 38, or at least 39, or at least 40, or at least 41, or at least 42, or at least 43, or at least 44, or at least 45, or at least 46, or at least 47, or at least 48, or at least 49, or at least 50, or at least 51, or at least 52, or at least 53, or at least 54, or at least 55, or at least 56, or at least 57, or at least 58, or at least 59, or at least 60, or at least 61, or at least 62, or at least 63, or at least 64, or at least 65, or at least 66, or at least 67, or at least 68, or at least 69, or at least 70, or at least 71, or at least 72, or at least 73, or at least 74, or at least 75, or at least 76, or at least 77, or at least 78, or at least 79, or at least 80, or at least 81, or at least 82, or at least, 83, or at least 84, or at least 85, or at least 86, or at least 87, or at least 88, or at least 89, or at least 90, or at least 91, or at least 92, or at least 93, or at least 94, or at least 95, or at least 96, or at least 97, or each of the biomarkers in the at least one biomarker signature.

In some aspects, an upregulated biomarker can be selected from DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22, and PML.

In some aspects, an upregulated biomarker can be selected from DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5, and BATF2.

In some aspects, an upregulated biomarker can be selected from AQP9.

In some aspects, a downregulated biomarker can be RFLNB.

In some aspects, a downregulated biomarker can be DEFA1.

In some aspects, a downregulated biomarker can be selected from AQP1, AQP3, AQP4, AQP4-AS1, AQP5 and AQP7

The following are non-limiting examples of methods of the present disclosure based on the methods and biomarker signatures described above.

### General Methods and Definitions

Any of the following general methods and definitions can be applied to any of the methods and associated biomarker signatures described above.

In some aspects of the present disclosure, a biomarker can be an mRNA. In some aspects of the present disclosure, a biomarker can be a long intervening/intergenic non-coding RNA (lincRNA).

In each of the preceding methods, and any of the methods described herein, in addition to analyzing the expression level of the at least one biomarker in microvesicular RNA isolated from a saliva sample, the expression level of the at least one biomarker can be analyzed in both microvesicular RNA and cell-free DNA from a saliva sample from the subject. Thus, in a non-limiting example, the present disclosure provides a method of identifying the presence or absence of Sjögren's syndrome in a subject, the method comprising analyzing the expression level of at least one biomarker selected from at least one biomarker signature in microvesicular RNA and cell-free DNA (cfDNA) isolated from a saliva sample from the subject.

In some aspects, any method of the present disclosure can further comprise administering at least one treatment to a subject identified as having Sjögren's syndrome.

In some aspects, any method of the present disclosure, prior to step (a), can further comprise: i) isolating a plurality of microvesicles from a saliva sample from the subject and ii) extracting at least one microvesicular RNA from the plurality of isolated microvesicles.

In some aspects, any method of the present disclosure, prior to step (a), can further comprise: i) isolating a microvesicle fraction from a saliva sample from the subject, wherein the microvesicle fraction comprises a plurality of microvesicles and cfDNA: ii) extracting at least one microvesicular RNA and at least one cfDNA molecule from the isolated microvesicle fraction.

In some aspects, prior to the isolation of microvesicles, RNAse inhibitor can be added to a saliva sample.

In some aspects, RNase inhibitor is added to the saliva sample with at least about 1 minute, or at least about 1 hour, or at least about 24 hours of collecting the saliva sample.

In some aspects of the methods of the present disclosure, isolating a plurality of microvesicles from a biological sample from the subject can comprise a processing step to remove cells, cellular debris or a combination of cells and cellular debris. A processing step can comprise filtering the sample, centrifuging the sample, or a combination of filtering the sample and centrifuging the sample. Centrifuging can comprise centrifuging at about 2000×g. Filtering can comprise filtering the sample through a filter with a pore size of about 0.8 microns.

In some aspects of the methods of the present disclosure, isolating a plurality of microvesicles can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating a microvesicle fraction, wherein the microvesicle fraction comprises a plurality of microvesicles and cfDNA can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating an at least one microvesicle is from a saliva sample can comprise contacting the saliva sample with at least one affinity agent that binds to at least one surface marker present on the surface the at least one microvesicle.

In some aspects, the microvesicular RNA can be isolated from a saliva sample using an extraction-free method. In some aspects, the extraction-free method comprises direct lysis of microvesicles in the saliva sample without prior isolation of the microvesicles to yield microvesicular RNA. Without wishing to be bound by theory, the extraction-free method that does not include a microvesicle isolation step is more easily adapted to automated methods, particular for high-throughput sample processing. In some aspects, an extraction-free method can comprise directly adding a lysis solution to a saliva sample.

Other microvesicle and microvesicle fraction isolation procedures are described in US 2017-0088898 A1, US 2016-0348095 A1, US 2016-0237422 A1, US 2015-0353920 A1, U.S. Pat. No. 10,465,183 and US 2019-0284548 A1, the contents of each of which are incorporated herein by reference in their entireties. The methods of the present disclosure can comprise any of the methods described in the aforementioned United States patenttent Publications and United States patents.

Other microvesicle and microvesicle fraction isolation procedures are described in WO 2018/076018, the contents of which are incorporated herein by reference in their entireties. The methods of the present disclosure can comprise any of the methods described in the aforementioned PCT Application Publication.

In some aspects of the methods of the present disclosure, determining the expression level of a biomarker can comprise quantitative PCR (qPCR), quantitative real-time PCR, semi-quantitative real-time PCR, digital PCR (dPCR), reverse transcription PCR (RT-PCR), reverse transcription quantitative PCR (qRT-PCR), microarray analysis, sequencing, next-generation sequencing (NGS), high-throughput sequencing, direct-analysis or any combination thereof.

In some aspects of the methods of the present disclosure, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

In some aspects of the methods of the present disclosure, an expression level of a biomarker or endogenous control gene can correspond to a cycle threshold (Ct) value when the expression level is determined using quantitative PCR (qPCR), quantitative real-time PCR, semi-quantitative real-time PCR, reverse transcription PCR (RT-PCR) or reverse transcription quantitative PCR (qRT-PCR).

In some aspects, any of the expression levels of a biomarker can be normalized using methods known in the art. For example, expression levels of biomarkers measured in the methods disclosed herein can be normalized to the expression level of an endogenous control gene and/or a reference biomarker. In some aspects, normalizing the expression level of a biomarker to the expression level of an endogenous control gene and/or a reference biomarker can comprise subtracting the expression level of the endogenous control gene and/or a reference biomarker from the expression level of the biomarker. Accordingly, in aspects wherein the expression levels are measured as Ct values, the normalized expression value of a biomarker can be the Ct value of the biomarker minus the Ct value of the endogenous control gene and/or a reference biomarker. In some aspects, normalizing the expression level of a biomarker to the expression level of an endogenous control gene can comprise dividing the expression level of the biomarker by the expression level of the endogenous control gene and/or a reference biomarker.

In some aspects of the methods of the present disclosure wherein determining the expression level of a biomarker comprises sequencing, next-generation sequencing (NGS), high-throughput sequencing, or any combination thereof, at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, or any combination thereof can correspond to subject's transcriptome.

In some aspects of the methods of the present disclosure, microvesicular RNA and/or cell-free DNA that has been extracted from a plurality of isolated microvesicles and/or an isolated microvesicle fraction, and/or isolated from an extraction-free process can be subjected to library preparation procedures that are known in the art for the preparation of a library for sequencing, including next-generation sequencing and/or high-throughput sequencing.

In some aspects, microvesicular RNA and/or cfDNA isolated from a plurality of isolated microvesicles or a microvesicle fraction may be further processed in one or more steps, as described herein. These one or more steps can be performed concurrently or in any order.

Extracted microvesicular RNA can be further processed by fragmentation. Extracted cfDNA can be further processed by fragmentation. In some aspects, fragmentation can be performed at about 85° C. In some aspects, fragmentation can be performed for about 1 minute, or about 2 minutes, or about 3 minutes.

Extracted microvesicular RNA can be further processed by contacting the extracted microvesicular RNA with solid-phase reversible immobilization (SPRI) beads. Extracted cfDNA can be further processed by contacting the extracted microvesicular RNA with solid-phase reversible immobilization (SPRI) beads.

Extracted microvesicular RNA can be amplified using PCR. In some aspects amplification by PCR can be performed for about 18 cycles.

Extracted microvesicular RNA can be further processed to selectively remove ribosomal DNA and/or RNA sequences from the extracted microvesicular RNA. In some aspects, selectively removing ribosomal DNA and/or RNA sequences can comprise the use of enzymatic reagents, including, but not limited to, RNase H or any other restriction enzyme. In some aspects, selectively removing ribosomal DNA and/or RNA sequences can comprise contacting the extracted microvesicular RNA with at least one affinity agent that binds to the ribosomal DNA and/or sequences. In some aspects, selectively removing ribosomal DNA and/or RNA sequences can comprise: i) contacting the extracted microvesicular RNA with biotinylated probes that hybridize to ribosomal DNA and/or RNA sequences; and ii) removing the hybridized probes using streptavidin conjugated paramagnetic beads.

Extracted cfDNA can be further processed to selectively remove ribosomal DNA and/or RNA sequences from the extracted cfDNA. In some aspects, selectively removing ribosomal DNA and/or RNA sequences can comprise the use of enzymatic reagents, including, but not limited to, RNase H or any other restriction enzyme. In some aspects, selectively removing ribosomal DNA and/or RNA sequences can comprise contacting the extracted cfDNA with at least one affinity agent that binds to the ribosomal DNA and/or sequences. In some aspects, selectively removing ribosomal DNA and/or RNA sequences can comprise: i) contacting the extracted cfDNA with biotinylated probes that hybridize to ribosomal DNA and/or RNA sequences; and ii) removing the hybridized probes using streptavidin conjugated paramagnetic beads.

Extracted microvesicular RNA can be further processed to reverse transcribe the extracted microvesicular RNA into cDNA. Reverse transcription can be performed using methods known in the art.

cDNA and/or cfDNA can be further processed to construct a double-stranded DNA sequencing library from the reverse transcribed cDNA and/or cfDNA. The double-stranded DNA sequencing library can be further amplified prior to sequencing. In some aspects, the amplification can be performed using PCR. In some aspects, the PCR amplification of the library can be performed for about 17 cycles, or about 18 cycles, or about 19 cycles. In some aspects, the PCR amplification can be performed for about 18 cycles.

In some aspects, the amplification can be selective amplification of at least one biomarker. Selective amplification can be performed by PCR, wherein the PCR comprises the use of PCR primers that selectively hybridize to the at least one biomarker. A double-stranded DNA sequencing library or an amplified double-stranded DNA sequencing library can further comprise selectively enriching for at least one biomarker from the double-stranded DNA sequencing library or the amplified double-stranded DNA sequencing library. Selectively enriching at least one biomarker from the double-stranded DNA sequencing library or the amplified double-stranded DNA sequencing library can comprise the use of hybrid capture methods known in the art. Hybrid capture methods can comprise contacting the double-stranded DNA sequencing library or the amplified double-stranded DNA sequencing library with at least one affinity agent that binds to the at least one biomarker to be enriched. In a non-limiting example, selectively enriching at least one biomarker from the double-stranded DNA sequencing library or the amplified double-stranded DNA sequencing library can comprise: i) contacting the double-stranded DNA sequencing library or the amplified double-stranded DNA sequencing library with at least one biotinylated probe that binds to the at least one biomarker; and ii) enriching the hybridized probes using streptavidin conjugated paramagnetic beads.

cDNA can be further processed to amplify the cDNA. The amplification can be selective amplification of at least one biomarker. Selective amplification can be performed by PCR, wherein the PCR comprises the use of PCR primers that selectively hybridize to the at least one biomarker. cDNA or amplified cDNA can be further processed to selectively enrich at least one biomarker. Selectively enriching at least one biomarker from cDNA or amplified cDNA can comprise the use of hybrid capture methods known in the art. The hybrid capture methods can comprise contacting the cDNA or amplified cDNA with at least one affinity agent that binds to the at least one biomarker to be enriched. In a non-limiting example, selectively enriching at least one biomarker from cDNA or amplified cDNA can comprise: i) contacting the cDNA or amplified cDNA with at least one biotinylated probe that binds to the at least one biomarker; and ii) enriching the hybridized probes using streptavidin conjugated paramagnetic beads.

cfDNA can be further processed to amplify the cfDNA. The amplification can be selective amplification of at least one biomarker. Selective amplification can be performed by PCR, wherein the PCR comprises the use of PCR primers that selectively hybridize to the at least one biomarker. cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads.

Extracted microvesicular RNA can be further processed to amplify the extracted microvesicular RNA. The amplification can be selective amplification of at least one biomarker. Selective amplification can be performed by PCR, wherein the PCR comprises the use of PCR primers that selectively hybridize to the at least one biomarker. Extracted microvesicular RNA or amplified microvesicular RNA can be further processed to selectively enrich at least one biomarker. Selectively enriching at least one biomarker from extracted microvesicular RNA or amplified microvesicular RNA can comprise the use of hybrid capture methods known in the art. The hybrid capture methods can comprise contacting the extracted microvesicular RNA or amplified microvesicular RNA with at least one affinity agent that binds to the at least one biomarker to be enriched. In a non-limiting example, selectively enriching at least one biomarker from extracted microvesicular RNA or amplified microvesicular RNA can comprise: i) contacting the extracted microvesicular RNA or amplified microvesicular RNA with at least one biotinylated probe that binds to the at least one biomarker; and ii) enriching the hybridized probes using streptavidin conjugated paramagnetic beads.

In some aspects, determining the expression level of at least one biomarker in microvesicular RNA, or in a mixture of microvesicular RNA and cfDNA can comprise fragmenting the microvesicular RNA.

In some aspects, determining the expression level of at least one biomarker in microvesicular RNA, or in a mixture of microvesicular RNA and cfDNA, can comprise reverse-transcribing the microvesicular RNA into cDNA. In some aspects, the cDNA can be amplified. The amplification can be a selective amplification of at least one biomarker. In some aspects, cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads. In some aspects, following the selective enrichment of at least one biomarker, the enriched at least one biomarker can be amplified. The amplification can be a selective amplification of the at least one biomarker.

In some aspects, the cDNA or amplified cDNA can be used to construct a double-stranded DNA sequencing library using techniques known in the art. cDNA or amplified cDNA that has been selectively enriched for at least one biomarker can be used to construct a double-stranded DNA sequencing library. cDNA or amplified cDNA that has been selectively enriched for at least one biomarker and then amplified again can be used to construct a double-stranded DNA sequencing library. Constructing a double-stranded DNA sequencing can be performed using methods known in the art.

In some aspects, determining the expression level of at least one biomarker in cfDNA, or in a mixture of microvesicular RNA and cfDNA, can comprise fragmenting the cfDNA.

In some aspects, determining the expression level of at least one biomarker in microvesicular RNA, or in a mixture of microvesicular RNA and cfDNA, can comprise amplifying the cfDNA. The amplification can be a selective amplification of at least one biomarker. In some aspects, cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads. In some aspects, following the selective enrichment of at least one biomarker, the enriched at least one biomarker can be amplified. The amplification can be a selective amplification of the at least one biomarker.

In some aspects, the cfDNA or amplified cfDNA can be used to construct a double-stranded DNA sequencing library using techniques known in the art. cfDNA or amplified cfDNA that has been selectively enriched for at least one biomarker can be used to construct a double-stranded DNA sequencing library. cfDNA or amplified cfDNA that has been selectively enriched for at least one biomarker and then amplified again can be used to construct a double-stranded DNA sequencing library. Constructing a double-stranded DNA sequencing can be performed using methods known in the art.

In some aspects of the methods of the present disclosure, the hybrid-capture methods described herein substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome. In some aspects of the methods of the present disclosure, the hybrid-capture methods described herein result in a significant depletion in microbial nucleic acids.

In some aspects, any of the methods described herein can be performed in an automation-compatible instrument. Automation-compatible instruments include, but are not limited to, Tecan liquid handling device, a Hamilton liquid handling device, or any other platforms capable of performing high-throughput specimen processing in a research or diagnostic setting.

The present disclosure provides a method of purifying nucleic acid transcripts that correspond to the human transcriptome from a saliva sample from a human subject, the method comprising: a) isolating a plurality of microvesicles from the saliva sample; b) extracting microvesicular RNA from the plurality of isolated microvesicles; c) purifying nucleic acid transcripts that correspond to the human transcriptome from the extracted microvesicular RNA by performing hybrid-capture, wherein the product of the hybrid-capture is substantially enriched for nucleic acid transcripts that correspond to the human transcriptome and is substantially depleted of nucleic acids that are derived from microbes. In some aspects, the product of the hybrid-capture can comprise at least about 70%, or at least about 75%, or at least about 80%, or at least about 85%, or at least about 90%, or at least about 95%, or at least about 99%, or at least about 99.5% nucleic acid transcripts that correspond to the human transcriptome. In some aspects, the product of the hybrid-capture can comprise no more than about 25%, or about 20%, or about 15%, or about 10%, or about 5%, or about 2.5%, or about 1%, or about 0.5% nucleic acid transcripts that are derived from a microbe.

The present disclosure provides a method of purifying nucleic acid transcripts that correspond to the human transcriptome from a saliva sample from a human subject, the method comprising: a) isolating a plurality of microvesicles and cfDNA from the saliva sample; b) extracting microvesicular RNA from the plurality of isolated microvesicles; c) purifying nucleic acid transcripts that correspond to the human transcriptome from the extracted microvesicular RNA and isolated cfDNA by performing hybrid-capture, wherein the product of the hybrid-capture is substantially enriched for nucleic acid transcripts that correspond to the human transcriptome and is substantially depleted of nucleic acids that are derived from microbes. In some aspects, the product of the hybrid-capture can comprise at least about 70%, or at least about 75%, or at least about 80%, or at least about 85%, or at least about 90%, or at least about 95%, or at least about 99%, or at least about 99.5% nucleic acid transcripts that correspond to the human transcriptome. In some aspects, the product of the hybrid-capture can comprise no more than about 25%, or about 20%, or about 15%, or about 10%, or about 5%, or about 2.5%, or about 1%, or about 0.5% nucleic acid transcripts that are derived from a microbe.

In some aspects, the subject is human.

In some aspects, the subject can have been previously diagnosed with Sjögren's syndrome based on the presence of anti-Ro autoantibody (also referred to as Anti-Sjögren's syndrome-related antigen A autoantibodies [anti-SSA]) in at least one biological sample from the subject.

In some aspects, the subject can have been previously diagnosed with SSA positive Sjögren's syndrome based on the presence of anti-Ro autoantibody (also referred to as Anti-Sjögren's syndrome-related antigen A autoantibodies [anti-SSA]) in at least one biological sample from the subject.

In some aspects, the subject can have been previously identified as anti-Ro autoantibody negative based on the absence of the anti-Ro autoantibody in at least one biological sample from the subject.

In some aspects, the subject can have been previously diagnosed with Sjögren's syndrome based on the presence of anti-La autoantibody (also referred to as Anti-Sjögren's syndrome-related antigen B autoantibodies [anti-SSB]) in at least one biological sample from the subject.

In some aspects, the subject can have previously undergone a lip biopsy.

In some aspects, the subject can have been previously identified as anti-Ro autoantibody negative based on the absence of the anti-Ro autoantibody in at least one biological sample from the subject, but can have been diagnosed with SSA negative Sjögren's syndrome based on the results of a lip biopsy.

In some aspects, the subject can have been previously identified as anti-La autoantibody negative based on the absence of the anti-La autoantibody in at least one biological sample from the subject.

In some aspects, the methods described herein can be used in combination with standard lip biopsy methods currently used for the diagnosis of Sjögren's syndrome.

In some aspects, the methods described herein can be used in combination with anti-Ro autoantibody (also referred to as Anti-Sjögren's syndrome-related antigen A autoantibodies [anti-SSA]) assays and/or the results from such anti-Ro autoantibody assays.

In some aspects, the methods described herein can be used in combination with anti-La autoantibody (also referred to as Anti-Sjögren's syndrome-related antigen B autoantibodies [anti-SSB]) assays and/or the results from such anti-La autoantibody assays.

In some aspects, the method described herein can be used in combination with methods based on one or more proteomic signatures derived for the diagnosis, monitoring or prognosis of Sjögren's syndrome. In some aspects, the proteomic signatures are derived from proteins extracted from microvesicles isolated from saliva samples from a subject.

In some aspects, the method described herein can be used in combination with methods based on one or more microbiome signatures derived for the diagnosis, monitoring or prognosis of Sjögren's syndrome. In some aspects, the microbiome signatures are derived from microbes isolated from saliva samples from a subject.

In some aspects, the methods described herein can be used in combination with methods based on one or more genetic signatures derived for the diagnosis, monitoring or prognosis of Sjögren's syndrome. Exemplary genetic signatures can include, but are not limited to, genetic signatures based on HLA genes, the IRF5 gene and STAT4 gene (see Imgenberg-Kreuz J, Rasmussen A, Sivils K, Nordmark G. Genetics and epigenetics in primary Sjögren's syndrome. Rheumatology. 2021 May 14; 60 (5): 2085-2098. doi: 10.1093/rheumatology/key330. PMID: 30770922; PMCID: PMC8121440, incorporated herein by reference in its entirety for all purposes).

In some aspects of the methods of the present disclosure, a predetermined cutoff value can be selected to have a negative predictive value (NPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a positive predictive value (PPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a sensitivity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a specificity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, an algorithm can be the product of a feature selection wrapper algorithm. In some aspects of the methods of the present disclosure, an algorithm can be the product of a machine learning algorithm. In some aspects of the methods of the present disclosure, an algorithm can be the product of a trained classifier built from at least one predictive classification algorithm. In some aspects of the methods of the present disclosure, an algorithm can be the product of a of a logistic regression model. A logistic regression model can comprise LASSO regularization.

In some aspects of the methods of the present disclosure a predictive classification algorithm, a feature selection wrapper algorithm, and/or a machine learning algorithm can comprise XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta (see Kursa M B, Rudnicki W R. Feature Selection with the Boruta Package. J Stat Softw 2010; 36 (11), incorporated herein by reference in its entirety) or any combination thereof.

In some aspects of the methods of the present disclosure, a predetermined cutoff value can be calculated using at least one receiver operating characteristic (ROC) curve. In some aspects of the methods of the present disclosure, a predetermined cutoff value can be calculated and/or selected to have any of the features described herein (e.g., a specific sensitivity, specificity, PPV, NPV or any combination thereof) using any method known in the art, as would be appreciated by the skilled artisan.

In some aspects of the methods of the present disclosure, an algorithm can a product of a feature selection wrapper algorithm, machine learning algorithm, trained classifier, logistic regression model or any combination thereof, that was trained to identify Sjögren's syndrome in a subject using: a) the expression levels of the at least one biomarker selected from the at least one biomarker signature in at least one biological sample from at least one subject who does not have Sjögren's syndrome; and b) the expression levels of the at least one biomarker selected from the at least one biomarker signature in at least one biological sample from at least one subject who has Sjögren's syndrome. In some aspects, the biological sample(s) is/are saliva samples.

In some aspects of the methods of the present disclosure, an algorithm can a product of a feature selection wrapper algorithm, machine learning algorithm, trained classifier, logistic regression model or any combination thereof, that was trained to identify Sjögren's syndrome in a subject using: a) the expression levels of the at least one biomarker selected from the at least one biomarker signature in at least one biological sample from at least one subject who does not have Sjögren's syndrome; b) the expression levels of the at least one biomarker selected from the at least one biomarker signature in at least one biological sample from at least one subject who has Sjögren's syndrome; c) the expression levels of the at least one biomarker selected from the at least one biomarker signature in at least one biological sample from at least one subject who has SSA positive Sjögren's syndrome, d) the expression levels of the at least one biomarker selected from the at least one biomarker signature in at least one biological sample from at least one subject who has SSA negative Sjögren's syndrome; e) the expression levels of the at least one biomarker selected from the at least one biomarker signature in at least one biological sample from a subject who does not have Sjögren's syndrome and who does not exhibit sicca symptoms; f) the expression levels of the at least one biomarker selected from the at least one biomarker signature in at least one biological sample from a subject who does not have Sjögren's syndrome but exhibits sicca symptoms; g) the expression levels of the at least one biomarker selected from the at least one biomarker signature in at least one biological sample from a subject who has at least on alternative disease/disorder; or h) any combination thereof. In some aspects, the biological sample(s) is/are saliva samples.

In some aspects, an alternative disease/disorder can be Systemic Lupus Erythematosus (SLE).

In some aspects, an alternative disease/disorder can be Rheumatoid Arthritis.

As would be appreciated by the skilled artisan, sicca symptoms include, but are not limited to dry eyes and dry mouth.

In some aspects, a predetermined cutoff value can be the expression level of a biomarker in a biological sample collected from a subject who does not have Sjögren's syndrome. In some aspects, a predetermine cutoff value can be the mean (average) expression level of a biomarker from a plurality of samples collected from a plurality of subjects who do not have Sjögren's syndrome.

In some aspects, a predetermined cutoff value can be the expression level of a biomarker in a biological sample collected from a subject who has Sjögren's syndrome. In some aspects, a predetermine cutoff value can be the mean (average) expression level of a biomarker from a plurality of samples collected from a plurality of subjects who have Sjögren's syndrome.

A treatment can comprise at least one therapeutically effective amount of an artificial tear, cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof.

A treatment can comprise at least one therapeutically effective amount of UCB5857 (targeting PI3K8 by selectively inhibiting PI3K8 preventing transmission of cell surface receptor signaling); CFZ533 (targeting CD40 by being Fc silent antibody to CD40 preventing B cell stimulation and differentiation without depletion); AMG557 (targeting ICOS by inhibiting activation of TFH); VAY736 (ianalumab; targeting BAFF-R by being an antibody to BAFF-R preventing BAFF-mediated B cell proliferation and survival); IL-2 (targeting CD4⁺CD25⁺ T cells by expanding Treg cells); a combination of rituximab and belimumab (targeting CD20 B cells and BAFF by eliciting anti-CD20-dependent depletion of B cells combined with BAFF blockade to decrease survival of self-reactive B cells); tocilizumab (targeting IL-6R by causing blockade of IL-6R preventing IL-6-dependent TH17 and TFH cell differentiation); abatacept (targeting CD80/86 through CTLA4-Ig binding of CD80/86 prevents co-stimulation-dependent activation of CD4 T cells); RSLV-132 (a fully human biologic Fc fusion protein, targeting extracellular RNA to prevent activation of the immune system via Toll-like receptors and the interferon pathway); VIB4920 (a fusion protein binding to CD40L on activated T cells, blocking their interaction with CD40-expressing B cells), iscalimab (anti-CD40); baricitinib (JAK1/2 inhibitor); nipocalimab (CD40L blockade); dazodalibep (CD40L blockade), MHV370; S95011 (OSE-127; anti-IL 7R antibody); efgartigimod (anti-FcRn antibody); tofacitinib (JAK inhibitor); iguratomid (NF-κB blocker); anifrolumab (anti-IFNAR antibody); branebrutinib (BTK inhibitor); telitacicept (BAFF/APRIL neutralization), SAR441344; or any combination thereof.

A treatment can comprise surgery. A surgery can comprise a surgery to seal the tear ducts that drain tears from the subject's eyes (also referred to as a punctal occlusion). The tear ducts may be sealed, for example, by inserting collagen or silicone plugs into the ducts.

A treatment can be a gene therapy-based treatment. In some aspects, a gene therapy-based treatment can comprise the administration of at least one Adeno-associated virus (AAV)-based therapy. In some aspects, the AAV-based therapy comprises administering to a subject a therapeutically effective amount of an AAV-based vector comprising a nucleic acid sequence encoding at least one aquaporin protein, or a functional fragment thereof. In some aspects, the at least one aquaporin protein can be selected from AQP1, AQP3, AQP4, AQP5, AQP7 and AQP9. In some aspects the at least one aquaporin protein is AQP1. In some aspects, the at least one aquaporin protein is AQP5.

In some aspects, a treatment can comprise and of the treatments are described in Suzanne Arends et al. (2023), Expert Review of Clinical Immunology; the contents of which are incorporated herein by reference in their entireties.

In some aspects of the methods of the present disclosure, a saliva sample can be collected at the subject's home through the use of a sample home-collection device.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

In some aspects, the methods of the present disclosure, or portions thereof, can be performed within the home of the subject. In some aspects of the methods of the present disclosure, the saliva sample is collected at the home of the subject.

In some aspects, the methods of the present disclosure, or portions thereof can be performed at about room temperature.

In some aspects, the samples used in the methods of the present disclosure can be stored at about 4° C. for any length of time. In some aspects, the samples used in the methods of the present disclosure can be stored at about −20° C. for any length of time. In some aspects, the samples used in the methods of the present disclosure can be stored at about −80° C. for any length of time.

### Kits of the Present Disclosure

The present disclosure provides kits comprising plurality of agents specific to detect the expression levels of one or more biomarkers selected from one or more of the biomarker signatures described herein. In this way, the kit can be used in combination with any of the methods described herein to effectuate the method using a saliva sample collected from a subject. That is, the kits of the present disclosure can be used to identify the presence or absence of Sjögren's syndrome, monitoring a Sjögren's syndrome treatment in a subject, and treating Sjögren's syndrome in a subject using the methods described herein.

In some aspects of the kits of the present disclosure, the plurality of agents specific to detect the expression levels of one or more biomarkers can be oligonucleotide primers. In some aspects, the oligonucleotide primers can be labeled.

In some aspects of the kits of the present disclosure, the plurality of agents specific to detect the expression levels of one or more biomarkers can be antibodies, or antigen-binding fragments thereof. In some aspects, the antibodies, or antigen-binding fragments thereof, can be labeled.

The kits of the present disclosure can comprise a plurality of agents suitable for enriching one or more biomarkers selected from one or more of the biomarker signatures described herein. Agents suitable for enriching the one or more biomarkers include, but are not limited to, oligonucleotide probes that specifically bind to the one or more biomarkers and that comprise at least one affinity label. In a non-limiting example, agents suitable for enriching the one or more biomarkers include any reagents known in the art to be useful in nucleic acid hybrid capture methods.

Suitable labels include, but are not limited to fluorescent labels, calorimetric labels, radioactive labels or any other label known in the art.

Accordingly, the kits of the present disclosure can further provide instructions for performing the methods of the present disclosure.

The kits of the present disclosure can further comprise a saliva sample collection device. Any saliva sample collection device known in the art, including, but not limited to, saliva sample collection devices that allow a subject to collect a sample without the supervision of a medical professional (e.g., a sample home-collection device and/or a saliva home collection device/kit).

As would be appreciated by the skilled artisan, examples of sample home-collection devices and saliva home collection kits include, but are not limited to, DNA/RNA Shield SafeCollect Saliva Collection Kit (Zymo Research), SpeciMax™ Stabilized Saliva Collection Kit (ThermoFisher Scientific), PAxgene Saliva Collector (Qiagen), ORAcollect RNA (DNAGenotek), Saliva DNA Collection and Preservation Devices (Norgen Biotek), or any other saliva home collection kits known in the art. As would be appreciated by the skilled artisan, saliva home collection kits can comprise saliva home collection kits wherein the microvesicles present in the saliva sample are not lysed at the time of collection. As would be appreciated by the skilled artisan, any of the saliva home collection kits described above can be further supplemented with one or more aliquots of a sample stabilizing agents. As would be appreciated by the skilled artisan, any of the saliva home collection kits described above can be further supplemented with one or more aliquots of a microvesicular RNA stabilizing agent.

The kits of the present disclosure can further comprise a device for the isolation of exosomes using any of the methods described herein.

The kits of the present disclosure can further comprise one or more reagents for the extraction of microvesicular RNA, microvesicular cell-free DNA, microvesicular protein or any combination thereof from a saliva sample.

The kits of the present disclosure can further comprise one or more aliquots of an RNAse inhibitor.

The kits of the present disclosure can further comprise one or more aliquots of a sample stabilization agent. As would be appreciated by the skilled artisan, any sample stabilization agent known in the art would be suitable for use in the kits of the present disclosure.

### EXEMPLARY EMBODIMENTS

Embodiment 1a. A method of identifying the presence or absence of Sjögren's syndrome in a subject, the method comprising: a) determining the expression level of at least one biomarker selected from at least one biomarker signature in microvesicular RNA isolated from a saliva sample from the subject; and b) identifying the presence or absence of Sjögren's syndrome based on the expression level of the at least one biomarker.

Embodiment 1b. A method of identifying the risk of Sjögren's syndrome in a subject, the method comprising: a) determining the expression level of at least one biomarker selected from at least one biomarker signature in microvesicular RNA isolated from a saliva sample from the subject; and b) identifying the risk of Sjögren's syndrome based on the expression level of the at least one biomarker.

Embodiment 1c. A method of determining if a subject is Sjögren's syndrome positive or is Sjögren's syndrome negative: a) determining the expression level of at least one biomarker selected from at least one biomarker signature in microvesicular RNA isolated from a saliva sample from the subject; and b) identifying that the subject is Sjögren's syndrome positive or Sjögren's syndrome negative based on the expression level of the at least one biomarker.

Embodiment 1d. A method of monitoring a Sjögren's syndrome treatment in a subject that has been administered the Sjögren's syndrome treatment, the method comprising: a) determining the expression level of at least one biomarker selected from at least one biomarker signature in microvesicular RNA isolated from a saliva sample from the subject; and b) determining whether the subject is responding to the Sjögren's syndrome treatment based on the expression level of the at least one biomarker.

Embodiment 1e. A method of treating Sjögren's syndrome in a subject, the method comprising: a) determining the expression level of at least one biomarker selected from at least one biomarker signature in microvesicular RNA isolated from a saliva sample from the subject; and b) administering at least one treatment to the subject based on the expression level of the at least one biomarker selected from at least one biomarker signature.

Embodiment 1f. A method of identifying the presence or absence of SSA positive Sjögren's syndrome in a subject, the method comprising: a) determining the expression level of at least one biomarker selected from at least one biomarker signature in microvesicular RNA isolated from a saliva sample from the subject; and b) identifying the presence or absence of SSA positive Sjögren's syndrome based on the expression level of the at least one biomarker.

Embodiment 1g. A method of identifying the presence or absence of SSA negative Sjögren's syndrome in a subject, the method comprising: a) determining the expression level of at least one biomarker selected from at least one biomarker signature in microvesicular RNA isolated from a saliva sample from the subject; and b) identifying the presence or absence of SSA negative Sjögren's syndrome based on the expression level of the at least one biomarker.

Embodiment 1h. A method of distinguishing between SSA positive Sjögren's syndrome and SSA negative Sjögren's syndrome in a subject, the method comprising: a) determining the expression level of at least one biomarker selected from at least one biomarker signature in microvesicular RNA isolated from a saliva sample from the subject; and b) distinguishing between SSA positive Sjögren's syndrome and SSA negative Sjögren's syndrome based on the expression level of the at least one biomarker.

Embodiment 1i. The method of any one of the preceding embodiments, wherein identifying the presence or absence of Sjögren's syndrome based on the expression level of the at least one biomarker selected from at least one biomarker signature comprises comparing the one or more expression levels to corresponding predetermined cutoff value and determining the presence or absence of Sjögren's syndrome in the subject based on the relationship between the one or more expression levels and the corresponding predetermined cutoff values.

Embodiment 1j. The method of any one of the preceding embodiments, wherein identifying the risk of Sjögren's syndrome based on the expression level of the at least one biomarker selected from at least one biomarker signature comprises comparing the one or more expression levels to corresponding predetermined cutoff value and determining the risk of Sjögren's syndrome in the subject based on the relationship between the one or more expression levels and the corresponding predetermined cutoff values.

Embodiment 1k. The method of any one of the preceding embodiments, wherein identifying that the subject is Sjögren's syndrome positive or Sjögren's syndrome negative based on the expression level of the at least one biomarker selected from at least one biomarker signature comprises comparing the one or more expression levels to corresponding predetermined cutoff value and determining if the subject is Sjögren's syndrome positive or Sjögren's syndrome negative based on the relationship between the one or more expression levels and the corresponding predetermined cutoff values

Embodiment 1l. The method of any one of the preceding embodiments, wherein determining whether the subject is responding to the Sjögren's syndrome treatment based on the expression level of the at least on biomarker selected from at least one biomarker signature comprises comparing the one or more expression levels to predetermined cutoff values and determining if the subject is responding based on the relationship between the one or more expression levels and the corresponding predetermined cutoff values

Embodiment 1m. The method of any one of the preceding embodiments, wherein administering at least one treatment to the subject based on the expression level of the at least one biomarker selected from at least one biomarker signature further comprises comparing the one or more expression levels to corresponding predetermined cutoff values and determining if the treatment is needed based on the relationship between the one or more expression levels and the corresponding predetermined cutoff values.

Embodiment 1n. The method of any one of the preceding embodiments, wherein identifying the presence or absence of SSA positive Sjögren's syndrome based on the expression level of the at least one biomarker selected from at least one biomarker signature comprises comparing the one or more expression levels to corresponding predetermined cutoff value and determining the presence or absence of SSA positive Sjögren's syndrome in the subject based on the relationship between the one or more expression levels and the corresponding predetermined cutoff values

Embodiment 1o. The method of any one of the preceding embodiments, wherein identifying the presence or absence of SSA negative Sjögren's syndrome based on the expression level of the at least one biomarker selected from at least one biomarker signature comprises comparing the one or more expression levels to corresponding predetermined cutoff value and determining the presence or absence of SSA negative Sjögren's syndrome in the subject based on the relationship between the one or more expression levels and the corresponding predetermined cutoff values.

Embodiment 1p. The method of any one of the preceding embodiments, wherein distinguishing between SSA positive Sjögren's syndrome and SSA negative Sjögren's syndrome based on the expression level of the at least one biomarker selected from at least one biomarker signature comprises comparing the one or more expression levels to corresponding predetermined cutoff value; and distinguishing between SSA positive Sjögren's syndrome and SSA negative Sjögren's syndrome based on the relationship between the one or more expression levels and the corresponding predetermined cutoff values.

Embodiment 2a. A method of identifying the presence or absence of Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of Sjögren's syndrome based on the score.

Embodiment 2b. A method of identifying the risk of Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the risk of Sjögren's syndrome based on the score.

Embodiment 2c. A method of determining if a subject is Sjögren's syndrome positive or is Sjögren's syndrome negative, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying if the subject is Sjögren's syndrome positive or is Sjögren's syndrome negative syndrome based on the score.

Embodiment 2d. A method of monitoring a Sjögren's syndrome treatment in a subject that has been administered the Sjögren's syndrome treatment, the method comprising: a) determining the expression level of at least one biomarker selected from at least one biomarker signature in microvesicular RNA isolated from a saliva sample from the subject; b) inputting the expression levels from step (a) into an algorithm to generate a score; c) determining whether the subject is responding to the Sjögren's syndrome treatment based on the score.

Embodiment 2e. A method of treating Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) administering at least one treatment to the subject based on the score.

Embodiment 2f. A method of identifying the presence or absence of SSA positive Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of SSA positive Sjögren's syndrome based on the score.

Embodiment 2g. A method of identifying the presence or absence of SSA negative Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) identifying the presence or absence of SSA negative Sjögren's syndrome based on the score.

Embodiment 2h. A method of distinguishing between SSA positive Sjögren's syndrome and SSA negative Sjögren's syndrome in a subject, the method comprising: a) determining the expression level of at least one biomarker selected from at least one biomarker signature in microvesicular RNA isolated from a saliva sample from the subject; b) inputting the expression levels from step (a) into an algorithm to generate a score; c) distinguishing between SSA positive Sjögren's syndrome and SSA negative Sjögren's syndrome in the subject based on the score.

Embodiment 2i. The method of any one of the preceding embodiments, wherein identifying the presence or absence of Sjögren's syndrome based on the score comprises comparing the score to a predetermined cutoff value and determining the presence or absence of Sjögren's syndrome in the subject based on the relationship between the score and the predetermined cutoff value.

Embodiment 2j. The method of any one of the preceding embodiments, wherein identifying the risk of Sjögren's syndrome based on the score comprises comparing the score to a predetermined cutoff value and determining the risk of Sjögren's syndrome in the subject based on the relationship between the score and the predetermined cutoff value.

Embodiment 2k. The method of any one of the preceding embodiments, wherein identifying if the subject is Sjögren's syndrome positive or is Sjögren's syndrome negative based on the score comprises comparing the score to a predetermined cutoff value and determining if the subject is Sjögren's syndrome positive or is Sjögren's syndrome negative based on the relationship between the score and the predetermined cutoff value.

Embodiment 2l. The method of any one of the preceding embodiments, wherein determining whether the subject is responding to the Sjögren's syndrome treatment based on the score comprises comparing the score to a predetermined cutoff value and determining if the subject is responding based on the relationship between the score and the predetermined cutoff value.

Embodiment 2m. The method of any one of the preceding embodiments, wherein administering at least one treatment to the subject based on the score further comprises comparing the score to a predetermined cutoff value and determining if the treatment is needed based on the relationship between the score and the predetermined cutoff value.

Embodiment 2n. The method of any one of the preceding embodiments, wherein identifying the presence or absence of SSA positive Sjögren's syndrome based on the score comprises comparing the score to a predetermined cutoff value and determining the presence or absence of SSA positive Sjögren's syndrome in the subject based on the relationship between the score and the predetermined cutoff value.

Embodiment 2o. The method of any one of the preceding embodiments, wherein identifying the presence or absence of SSA negative Sjögren's syndrome based on the score comprises comparing the score to a predetermined cutoff value and determining the presence or absence of SSA negative Sjögren's syndrome in the subject based on the relationship between the score and the predetermined cutoff value.

Embodiment 2p. The method of any one of the preceding embodiments, wherein distinguishing between SSA positive Sjögren's syndrome and SSA negative Sjögren's syndrome in the subject based on the score comprises comparing the score to a predetermined cutoff value and distinguishing between SSA positive Sjögren's syndrome and SSA negative Sjögren's syndrome in the subject based on the relationship between the score and the predetermined cutoff value.

Embodiment 3a. A method of identifying if a subject has Rheumatoid Arthritis (RA) or Sjögren's syndrome, the method comprising: a) determining the expression level of at least one biomarker selected from at least one biomarker signature in microvesicular RNA isolated from a saliva sample from the subject; b) identifying the subject as having either RA or Sjögren's syndrome based on the expression level(s) measured in step (a).

Embodiment 3b. A method of identifying if a subject has Rheumatoid Arthritis (RA) or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either RA or Sjögren's syndrome based on the score.

Embodiment 3c. The method of any one of the preceding embodiments, wherein identifying if a subject has Rheumatoid Arthritis (RA) or Sjögren's syndrome based on the expression level of the at least one biomarker selected from at least one biomarker signature comprises comparing the one or more expression levels to corresponding predetermined cutoff value; and identifying if a subject has Rheumatoid Arthritis (RA) or Sjögren's syndrome based on the relationship between the one or more expression levels and the corresponding predetermined cutoff values.

Embodiment 3d. The method of any one of the preceding embodiments, wherein identifying if a subject has Rheumatoid Arthritis (RA) or Sjögren's syndrome based on the score comprises comparing the score to a predetermined cutoff value; and identifying if a subject has Rheumatoid Arthritis (RA) or Sjögren's syndrome based on the relationship between the score and the predetermined cutoff value.

Embodiment 4a. A method of identifying if a subject has Systemic Lupus Erythematosus (SLE) or Sjögren's syndrome, the method comprising: a) determining the expression level of at least one biomarker selected from at least one biomarker signature in microvesicular RNA isolated from a saliva sample from the subject; b) identifying the subject as having either SLE or Sjögren's syndrome based on the expression level(s) measured in step (a).

Embodiment 4b. A method of identifying if a subject has Systemic Lupus Erythematosus (SLE) or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either SLE or Sjögren's syndrome based on the score.

Embodiment 4c. The method of any one of the preceding embodiments, wherein identifying if a subject has SLE or Sjögren's syndrome based on the expression level of the at least one biomarker selected from at least one biomarker signature comprises comparing the one or more expression levels to corresponding predetermined cutoff value; and identifying if a subject has SLE or Sjögren's syndrome based on the relationship between the one or more expression levels and the corresponding predetermined cutoff values.

Embodiment 4d. The method of any one of the preceding embodiments, wherein identifying if a subject has SLE or Sjögren's syndrome based on the score comprises comparing the score to a predetermined cutoff value; and identifying if a subject has SLE or Sjögren's syndrome based on the relationship between the score and the predetermined cutoff value.

Embodiment 5a. A method of identifying if a subject has non-Sjögren's syndrome related sicca symptoms or Sjögren's syndrome, the method comprising: a) determining the expression level of at least one biomarker selected from at least one biomarker signature in microvesicular RNA isolated from a saliva sample from the subject; b) identifying the subject as having either non-Sjögren's syndrome related sicca symptoms or Sjögren's syndrome based on the expression level(s) measured in step (a).

Embodiment 5b. A method of identifying if a subject has non-Sjögren's syndrome related sicca symptoms or Sjögren's syndrome, the method comprising: a) determining the expression level of at least one biomarker selected from at least one biomarker signature in microvesicular RNA isolated from a saliva sample from the subject; b) inputting the expression levels from step (a) into an algorithm to generate a score; and c) identifying the subject as having either non-Sjögren's syndrome related sicca symptoms or Sjögren's syndrome based on the score.

Embodiment 5c. The method of any one of the preceding embodiments, wherein identifying if a subject has non-Sjögren's syndrome related sicca symptoms or Sjögren's syndrome based on the expression level of the at least one biomarker selected from at least one biomarker signature comprises comparing the one or more expression levels to corresponding predetermined cutoff value; and identifying if a subject has non-Sjögren's syndrome related sicca symptoms or Sjögren's syndrome based on the relationship between the one or more expression levels and the corresponding predetermined cutoff values.

Embodiment 5d. The method of any one of the preceding embodiments, wherein identifying if a subject has non-Sjögren's syndrome related sicca symptoms or Sjögren's syndrome based on the score comprises comparing the score to a predetermined cutoff value; and identifying if a subject has non-Sjögren's syndrome related sicca symptoms or Sjögren's syndrome based on the relationship between the score and the predetermined cutoff value

Embodiment 6a. The method of any one of the preceding embodiments, wherein the at least one biomarker signature comprises: RSAD2, OAS3, OAS2, IFIT1, OAS1, IFIT5, ISG15, IFIT3, IFI6, DDX60, OASL, USP18, GRAMD1B, LY6E, TRIM38, IFI44L, SLC4A11, PML, MX1, EPSTI1, IFIH1, EIF2AK2, XAF1, IFIT2, TRIM22, RFLNB, RTP4, KPTN, IFITM1, TMEM123, LINC01473, OTOF, GPRC5C, ISY1-RAB43, ZBP1, DDX58, IFITM3, NT5C3A, CMPK2, TBC1D16, IFI16, SHISA5, SERPING1, SP100, HERC5, BATF2, SHC2, UBE2L6, GLIS2, ZC3HAV1, GRAMD1A, TNFSF10, APOBEC3F, SNHG15, PDCD4-AS1, TOX, VAMP5, ERICH1, MRAS, SAMHD1 and TP5313.

Embodiment 6b. The method of any one of the preceding embodiments, wherein the at least one biomarker signature comprises: DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22, RFLNB and PML.

Embodiment 6c. The method of any one of the preceding embodiments, wherein the at least one biomarker signature comprises: DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22 and PML.

Embodiment 6d. The method of any one of the preceding embodiments, wherein the at least one biomarker signature comprises: DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5, BATF2 and DEFA1.

Embodiment 6e. The method of any one of the preceding embodiments, wherein the at least one biomarker signature comprises: DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5 and BATF2.

Embodiment 6f. The method of any one of the preceding embodiments, wherein the at least one biomarker signature comprises: ISG15, RSAD2, TRIM38, and IFI6.

Embodiment 6g. The method of any one of the preceding embodiments, wherein the at least one biomarker signature comprises: TP5313, NT5C3A, SAMHD1, IFITM3, XAF1, GRAMD1A, SHC2, TBC1D16, ERICH1, OTOF, APOBEC3F, SP100, GLIS2, RTP4, SERPING1, TMEM123, EIF2AK2, HERC5, LINC01473, KPTN, IFITM1, IFIT2, DDX58, SHISA5, IFI44L, IFIT1, TNFSF10, UBE2L6, USP18, BATF2, VAMP5, OAS2, GPRC5C, ZBP1, SNHG15, TOX, LY6E, IFIT3, RFLNB, MX1, PML, TRIM22, IFI16, SLC4A11, EPSTI1, MRAS, ISG15, GRAMD1B, OAS1, OASL, TRIM38, IFI6, IFIT5, RSAD2, CMPK2, ZC3HAV1, IFIH1, OAS3 and DDX60.

Embodiment 6 h. The method of any one of the preceding embodiments, wherein the at least one biomarker signature comprises: IFIH1, DDX60, OAS3 and ZC3HAV1.

Embodiment 6i. The method of any one of the preceding embodiments, wherein the at least one biomarker signature comprises: RSAD2, IFI6, IFIT5 and CMPK2.

Embodiment 6j. The method of any one of the preceding embodiments, wherein the at least one biomarker signature comprises: DDX60, OAS3, IFI6 and RSAD2.

Embodiment 6k. The method of any one of the preceding embodiments, wherein the at least one biomarker signature comprises: CMPK2, OAS1, OASL and ISG15.

Embodiment 6l. The method of any one of the preceding embodiments, wherein the at least one biomarker signature comprises: IFIH1, ISG15, EPSTI1, IFI16, RSAD2 and OAS1.

Embodiment 6m. The method of any one of the preceding embodiments, wherein the at least one biomarker signature comprises: ISG15, IFI16, RSAD2 and OAS1.

Embodiment 6n. The method of any one of the preceding embodiments, wherein the at least one biomarker signature comprises: IFIH1, ISG15, EPSTI1 and IFI16.

Embodiment 6o. The method of any one of the preceding embodiments, wherein the at least one biomarker signature comprises: ISG15, RSAD2, IFI16 and OAS1.

Embodiment 6p. The method of any one of the preceding embodiments, wherein the at least one biomarker signature comprises: SERPING1, RTP4, SLC4A11 and MRAS.

Embodiment 6q. The method of any one of the preceding embodiments, wherein the at least one biomarker signature comprises: ISG15, IFIH1, EPSTI1 and IFI16.

Embodiment 6r. The method of any one of the preceding embodiments, wherein the at least one biomarker signature comprises: NT5C3A, IFIH1, RTP4 and IFI44L.

Embodiment 6s. The method of any one of the preceding embodiments, wherein the at least one biomarker signature comprises: ISG15, IFIH1, IFI16 and SLC4A11.

Embodiment 6t. The method of any one of the preceding embodiments, wherein the at least one biomarker signature comprises: AQP1, AQP3, AQP4, AQP4-AS1, AQP5 and AQP7.

Embodiment 6u. The method of any one of the preceding embodiments, wherein the at least one biomarker signature comprises: AQP9.

Embodiment 6v. The method of any one of the preceding embodiments, wherein the at least one biomarker signature comprises: AQP9 and AQP1.

Embodiment 6w. The method of any one of the preceding embodiments, wherein the at least one biomarker signature comprises: ISG15, IFI6, RXRA, IFIT1, STAT1, APOBEC3G, SP110, ERG, MORC3, IFI44L, MX1, SP100, LY6E, IFI44, ADAR, OAS1, IRF9, IFIT3, EIF2AK2, TGIF1, BST2, OAS2, CMTR1, UBE2L6, BRD3, IFI35 and IFI30.

Embodiment 6x. The method of any one of the preceding embodiments, wherein the at least one biomarker signature comprises: IFIH1, MX1, GBP1, TNFSF10, OAS1, IFIT1, IFIT5, IFI44L, CXCL10, IFIT3, OAS2, OASL, IFI16, STAT1, ZC3HAV1, TRIM22, RSAD2, IFITM1, IFI44, IFIT2, DDX58, DDX60, USP18, RTP4, SAMD9, HERC5, SAMD9L, CMPK2, CD274, EPSTI1 and DDX60L.

Embodiment 6y. The method of any one of the preceding embodiments, wherein the at least one biomarker signature comprises: PDZK1IP1, MYL9, SPON2, IFI44, IFI44L, LILRA3, CHI3L1, CMTM5, CLU, CMPK2, CMTM2, DTX3L, TYMP, EGR1, EGR2, APOBEC3A, SAMD9L, FCER1A, DDX58, IFIT5, IFI6, STAP1, GBP1, GGTA1, LAMP3, GP9, TRBV27, FFAR2, GZMB, TREML1, IFI27, IFI35, IFIT2, IFIT1, IFIT3, CXCL8, CXCL10, IRF7, ITGA2B, JUP, KCNJ15, KLRD1, ARG1, IFITM3P7, LGALS3BP, CYP4F3, LY6E, MMP9, MX1, MX2, OAS1, OAS2, OAS3, GOS2, LAP3, HERC5, MS4A4A, PLSCR1, XAF1, SAMD9, HERC6, TMEM140, DDX60, EIF2AK2, PROS1, CABP5, HES4, RPS23, CCL2, CCL8, RTP4, MMP25, PARP12, SIGLEC1, STAT1, SERPING1, C1QA, C1QB, TNFAIP6, C1QC, C3AR1, DHX58, ZBP1, TCL1A, PARP9, SH3BGRL2, OASL, PNPT1, MGAM, RSAD2, CD163, EPSTI1, APOBEC3B, ISG15 and SCO2.

Embodiment 7a. The method of any one of the preceding embodiments, wherein the at least one biomarker signature comprises: C19orf48, RNF26, NOS2, ZNF775, ANKRD29, OAS1, ARSL, LAMB1, and TUBB3, ARSL, CTSC, ZCCHC4, UGT2A1, IFIT1, CD101-AS1, ANKRD29, PRRX2, OAS1, MUC2, ARSL, NKX6-2, HTRA3, BSN, ZCCHC4, UGT2A1, IFIT1, and CD101-AS1.

Embodiment 7b. The method of any one of the preceding embodiments, wherein the at least one biomarker signature comprises: C19orf48, HNRNPA2B1, RNF26, ZNF542P, CHRFAM7A, ENSG00000285818, CENPO, POLR1G, RASL11A, RPRM, DDR2, SSPN, ACP2, ZNF688, PLEK2, CHST13, SERPINF2, NOS2, EFCAB12, LAMB1, FGF7P7, KLC4, ABCA13, RTKN2, TPPP, and BPIFB4.

Embodiment 7c. The method of any one of the preceding embodiments, wherein the at least one biomarker signature comprises: ZNF775, OAS1, TAF6L, MUC2, SLC17A9, OAS2, THBS1, ENSG00000260989, NOS2, EPSTI1, KPNB1, NRSN2, MX1, RSAD2, PARP9, S100A7, IFIT1, DPYSL4, ISG15, ANKRD29, PRRX2, TXLNB, RAD9B, ENSG00000276490, TNFRSF25 and SCAND2P.

Embodiment 7d. The method of any one of the preceding embodiments, wherein the at least one biomarker signature comprises: AP4E1, ARSL, TUBB3, CBARP, CRACDL, LAMB1, LHPP, ZNF846 and SLC35F3.

Embodiment 7e. The method of any one of the preceding embodiments, wherein the at least one biomarker signature comprises: ARSL and CTSC.

Embodiment 7f. The method of any one of the preceding embodiments, wherein the at least one biomarker signature comprises: DCUNID2, KCNC3, NRP2, IKZF4, OLFML2A, CNTN4-AS1, CCDC177, CARNS1, OR6B3, CEACAM22P, HECTD3, ENSG00000227678, NPR3, NOS3, SPAG5-AS1, FBF1, TRIM22, IFI44L, IFIT2, THBD, KPNB1, DOX60, ENSG00000259732, GBP5, OASL, SIX1, MX1, SCNM1, ENSG00000259345, OAS3, KLK14, LY6E, S100A7, IFIT3, IFI6, FMO2, RSAD2, NOS2, SPRR2F, IFIT1, ISG15, PARP9, ANOS2P, THBS1, TNFRSF25, HERC5, MUC2, TXLNB, OAS1, CHRFAM7A, EPSTI1, SLC17A9, OAS2, PRRX2, ANKRD29, and ENSG00000260989.

Embodiment 7g. The method of any one of the preceding embodiments, wherein the at least one biomarker signature comprises: OAS1, MUC2, NOS2, ENSG00000260989, OAS2, SLC17A9, SLC17A9, CHRFAM7A, EPSTI1, LY6E, THBS1, RSAD2, PARP9, IFI6, S100A7, OAS3, SPRR2F, IFIT1, HERC5, IFIT3, ANKRD29, ANOS2P, ISG15, TXLNB, PRRX2, TNFRSF25, FMO2, ENSG00000259345, and KLK14.

Embodiment 7h. The method of any one of the preceding embodiments, wherein the at least one biomarker signature comprises: SMAD4, ENSG00000279159, MAP3K15, DLC1, CB84, NOX4, CTSC, BSN, HTRA3, NKX6-2, and ARSL.

Embodiment 7i. The method of any one of the preceding embodiments, wherein the at least one biomarker signature comprises: ARSL, NKX6-2, CTSC, BSN and HTRA3.

Embodiment 7j. The method of any one of the preceding embodiments, wherein the at least one biomarker signature comprises: RNF157-AS1, MYO3A, LY6E, HECTD3, CDCA2, TRIM22, CCL17, DDX58, OTOF, RTP4, IGSF9, DDX60L, ISG15, RSAD2, RNF213, OASL, SIGLEC1, IFI44, IFIT2, OAS2, OAS3, IFI4L, IFIT5, IFIT3, CD101-AS1, OAS1, FAM111A-DT, IFIT1, MX1, HERC5, UGT2A1, and ZCCHC4.

Embodiment 7k. The method of any one of the preceding embodiments, wherein the at least one biomarker signature comprises: ZCCHC4, OAS1, IFIT5, IFI44L, MX1, HERC5, OAS2, IFIT3, IFIT1, FAM111A-DT, SIGLEC1, IFIT2, OAS3, IFI44, CD101-AS1, UGT2A1.

Embodiment 71. The method of any one of the preceding embodiments, wherein the at least one biomarker signature comprises: C19orf48, RNF26, and NOS2.

Embodiment 7m. The method of any one of the preceding embodiments, wherein the at least one biomarker signature comprises: ZNF775, ANKRD29, and OAS1.

Embodiment 7n. The method of any one of the preceding embodiments, wherein the at least one biomarker signature comprises: ARSL, LAMB1, and TUBB3.

Embodiment 7o. The method of any one of the preceding embodiments, wherein the at least one biomarker signature comprises: ARSL and CTSC.

Embodiment 7p. The method of any one of the preceding embodiments, wherein the at least one biomarker signature comprises: ZCCHC4, UGT2A1, IFIT1, and CD101-AS1.

Embodiment 7q. The method of any one of the preceding embodiments, wherein the at least one biomarker signature comprises: ANKRD29, PRRX2, OAS1, and MUC2.

Embodiment 7r. The method of any one of the preceding embodiments, wherein the at least one biomarker signature comprises: ARSL, NKX6-2, HTRA3, and BSN.

Embodiment 8a. The method of any one of the preceding embodiments, wherein, step (a) comprises determining the expression level of at least two, or at least three, or at least four, or at least five, or at least six, or at least seven, or at least eight, or at least nine, or at least 10, or at least 11, or at least 12, or at least 13, or at least four of the 14 biomarkers, or at least 15, or at least 16, or at least 17, or at least 18, or at least 19, or at least 20, or at least 21, or at least 22, or at least 23, or at least 24, or at least 25, or at least 26, or at least 27, or at least 28, or at least 29, or at least 30, or at least 31, or at least 32, or at least 33, or at least 34, or at least 35, or at least 36, or at least 37, or at least 38, or at least 39, or at least 40, or at least 41, or at least 42, or at least 43, or at least 44, or at least 45, or at least 46, or at least 47, or at least 48, or at least 49, or at least 50, or at least 51, or at least 52, or at least 53, or at least 54, or at least 55, or at least 56, or at least 57, or at least 58, or at least 59, or at least 60, or at least 61, or at least 62, or at least 63, or at least 64, or at least 65, or at least 66, or at least 67, or at least 68, or at least 69, or at least 70, or at least 71, or at least 72, or at least 73, or at least 74, or at least 75, or at least 76, or at least 77, or at least 78, or at least 79, or at least 80, or at least 81, or at least 82, or at least, 83, or at least 84, or at least 85, or at least 86, or at least 87, or at least 88, or at least 89, or at least 90, or at least 91, or at least 92, or at least 93, or at least 94, or at least 95, or at least 96, or at least 97, or each of the biomarkers in the at least one biomarker signature.

Embodiment 8b. The method of any one of the preceding embodiments, wherein, step (a) comprises determining the expression level of each of the biomarkers in the at least one biomarker signature.

Embodiment 9a. The method of any one of the preceding embodiments, wherein the algorithm is the product of a feature selection wrapper algorithm, a machine learning algorithm, a trained classifier built from at least one predictive classification algorithm or any combination thereof.

Embodiment 9b. The method of any one of the preceding embodiments, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

Embodiment 9c. The method of any one of the preceding embodiments, wherein the algorithm is the product of a feature selection wrapper algorithm, machine learning algorithm, trained classifier, logistic regression model or any combination thereof, that was trained to identify Sjögren's syndrome in a subject using: i) the expression level of the at least one biomarker in at least one biological sample from at least one subject who does not have Sjögren's syndrome; and ii) the expression levels of the at least one biomarker in at least one biological sample from at least one subject who has Sjögren's syndrome.

Embodiment 9d. The method of any one of the preceding embodiments, wherein the algorithm is the product of a feature selection wrapper algorithm, machine learning algorithm, trained classifier, logistic regression model or any combination thereof, that was trained to identify Sjögren's syndrome in a subject using: a) the expression levels of the at least one biomarker selected from the at least one biomarker signature in at least one biological sample from at least one subject who does not have Sjögren's syndrome; b) the expression levels of the at least one biomarker selected from the at least one biomarker signature in at least one biological sample from at least one subject who has Sjögren's syndrome; c) the expression levels of the at least one biomarker selected from the at least one biomarker signature in at least one biological sample from at least one subject who has SSA positive Sjögren's syndrome; d) the expression levels of the at least one biomarker selected from the at least one biomarker signature in at least one biological sample from at least one subject who has SSA negative Sjögren's syndrome; e) the expression levels of the at least one biomarker selected from the at least one biomarker signature in at least one biological sample from a subject who does not have Sjögren's syndrome and who does not exhibit sicca symptoms; f) the expression levels of the at least one biomarker selected from the at least one biomarker signature in at least one biological sample from a subject who does not have Sjögren's syndrome but exhibits sicca symptoms; g) the expression levels of the at least one biomarker selected from the at least one biomarker signature in at least one biological sample from a subject who has at least on alternative disease/disorder; or h) any combination thereof.

Embodiment 9e. The method of any one of the preceding embodiments, wherein the algorithm is the product of a trained classifier.

Embodiment 10. The method of any one of the preceding embodiments, wherein the saliva sample is collected using sample home-collection device.

Embodiment 11a. The method of any one of the preceding embodiments, further comprising prior to step (a): i) isolating a plurality of microvesicles from the saliva sample from the subject; and ii) extracting microvesicular RNA from the plurality of isolated microvesicles.

Embodiment 11b. The method of any one of the preceding embodiments, wherein prior to step (i), at least one stabilizing agent is added to the saliva sample, preferably wherein the at least one stabilizing agent is an RNAse inhibitor.

Embodiment 11c. The method of any one of the preceding embodiments, wherein the saliva samples are filtered, preferably wherein the saliva samples are filtered using a filter with an average pore size of about 0.8 μm.

Embodiment 11d. The method of any one of the preceding embodiments, further comprising fragmenting the extracted microvesicular RNA.

Embodiment 11e. The method of any one of the preceding embodiments, further comprising contacting the extracted microvesicular RNA with Solid-phase reversible immobilization (SPRI) beads.

Embodiment 11f. The method of any one of the preceding embodiments, wherein the extracted microvesicular RNA is amplified using PCR, preferably wherein the amplification is performed for about 18 cycles.

Embodiment 11g. The method of any one of the preceding embodiments, wherein the at least one microvesicle is isolated from the saliva sample by contacting the saliva sample with at least one affinity agent that binds to at least one surface marker present on the surface the at least one microvesicle.

Embodiment 12a. The method of any one of the preceding embodiments, wherein step (a) further comprises: (i) determining the expression level of at least one reference biomarker; (ii) normalizing the expression level of the at least one biomarker to the expression level of the at least one reference biomarker.

Embodiment 12b. The method of any one of the preceding embodiments, wherein inputting the expression levels from step (a) into an algorithm to generate a score comprises inputting the normalized expression levels from step (a) into an algorithm to generate a score.

Embodiment 13a. The method of any one of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

Embodiment 14a. The method of any one of the preceding embodiments, wherein determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

Embodiment 15a. The method of any one of the preceding embodiments, wherein the predetermined cutoff value has a negative predictive value of at least about 80%, or at least about 85%, or at least about 90%, or at least about 95%, or at least about 99%, or at least about 99.9%.

Embodiment 15b. The method of any one of the preceding embodiments, wherein the predetermined cutoff value has a positive predictive value of at least about 80%, or at least about 85%, or at least about 90%, or at least about 95%, or at least about 99%, or at least about 99.9%.

Embodiment 15c. The method of any one of the preceding embodiments, wherein the predetermined cutoff value has a sensitivity of at least about 80%, or at least about 85%, or at least about 90%, or at least about 95%, or at least about 99%, or at least about 99.9%.

Embodiment 15d. The method of any one of the preceding embodiments, wherein the predetermined cutoff value has a specificity of at least about 80%, or at least about 85%, or at least about 90%, or at least about 95%, or at least about 99%, or at least about 99.9%.

Embodiment 15e. The method of any one of the preceding embodiments, wherein the predetermined cutoff value is calculated using at least one receiver operating characteristic (ROC) curve.

Embodiment 16a. The method of any one of the preceding embodiments, wherein measuring expression levels in step (a) further comprises selectively enriching for the at least one biomarker.

Embodiment 16b. The method of any one of the preceding embodiments, wherein the at least one biomarker is selectively enriched by hybrid-capture, preferably wherein: i) the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome; and/or ii) the hybrid-capture results in a significant depletion in microbial nucleic acids

Embodiment 17a. The method of any one of the preceding embodiments, further comprising administering at least one treatment to a subject identified as having Sjögren's syndrome.

Embodiment 17b. The method of any one of the preceding embodiments, wherein the at least one treatment comprises: i) administering at least one therapeutically effective amount of an cevimeline, pilocarpine, a supersaturated calcium phosphate rinse, cyclosporine, tacrolimus eye drops, abatacept, rituximab, tocilizumab, hydroxypropyl cellulose, lifitegrast, LO2A eye drops, rebamipide eye drops, topical autologous serum, intravenous immunoglobulins, dexamethasone eye drops, an immunosuppressive medication, a nonsteroidal anti-inflammatory medication, an arthritis medication, an antifungal medication, hydroxychloroquine, alone or in combination with leflunomide, methotrexate, LOU064, INCB050465 or any combination thereof; ii) surgery, preferably wherein the surgery comprises sealing the tear ducts of the subject; iii) administering at least one therapeutically effective amount of UCB5857, CFZ533, AMG557, IL-2, a combination of rituximab and belimumab, tocilizumab, abatacept, RSLV-132, VIB4920, iscalimab, baricitinib, nipocalimab, dazodalibep, MHV370, S95011, efgartigimod, tofacitinib, iguratomid, anifrolumab, branebrutinib, telitacicept, SAR441344, or any combination thereof; iv) at least one AAV-based therapy, preferably wherein the at least one AAV-based therapy comprises an AAV-based vector comprising a nucleic acid sequence encoding at least one aquaporin protein, or a functional fragment thereof; or iv) a combination thereof.

Embodiment 18a. The method of any one of the preceding embodiments, wherein the Sjögren's syndrome is SSA positive Sjögren's syndrome.

Embodiment 18b. The method of any one of the preceding embodiments, wherein the Sjögren's syndrome is SSA negative Sjögren's syndrome.

Embodiment 19a. The method of any one of the preceding embodiments, wherein a subject that is identified as Sjögren's syndrome negative is a subject who does not have Sjögren's syndrome but has at least one alternative disease/disorder.

Embodiment 19b. The method of any one of the preceding embodiments, wherein the alternative disease/disorder causes the subject to exhibit one or more symptoms that are also symptoms of Sjögren's syndrome.

Embodiment 19c. The method of any one of the preceding embodiments, wherein a subject that is identified as Sjögren's syndrome negative is a subject who does not have Sjögren's syndrome but has sicca symptoms.

Embodiment 19d. The method of any one of the preceding embodiments, wherein a subject that is identified as Sjögren's syndrome negative is a subject who does not have Sjögren's syndrome and does not have at least one alternative disease/disorder, but has sicca symptoms.

Embodiment 19e. The method of any one of the preceding embodiments, wherein a subject that is identified as Sjögren's syndrome negative is a subject who does not have Sjögren's syndrome, does not have at least one alternative disease/disorder, and does not have sicca symptoms.

Embodiment 19f. The method of any one of the preceding embodiments, wherein sicca symptoms are dry eyes and/or dry mouth.

Embodiment 19g. The method of any one of the preceding embodiments, wherein

Embodiment 19h. The method of any one of the preceding embodiments, wherein the alternative disease/disorder is rheumatoid arthritis (RA).

Embodiment 19i. The method of any one of the preceding embodiments, wherein the alternative disease/disorder is Systemic Lupus Erythematosus (SLE).

### Additional Exemplary Embodiments

Embodiment 1a. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: RSAD2, OAS3, OAS2, IFIT1, OAS1, IFIT5, ISG15, IFIT3, IFI6, DDX60, OASL, USP18, GRAMD1B, LY6E, TRIM38, IFI44L, SLC4A11, PML, MX1, EPSTI1, IFIH1, EIF2AK2, XAF1, IFIT2, TRIM22, RFLNB, RTP4, KPTN, IFITM1, TMEM123, LINC01473, OTOF, GPRC5C, ISY1-RAB43, ZBP1, DDX58, IFITM3, NT5C3A, CMPK2, TBC1D16, IFI16, SHISA5, SERPING1, SP100, HERC5, BATF2, SHC2, UBE2L6, GLIS2, ZC3HAV1, GRAMD1A, TNFSF10, APOBEC3F, SNHG15, PDCD4-AS1, TOX, VAMP5, ERICH1, MRAS, SAMHD1, and TP5313; b) inputting the expression levels from step (a) into an algorithm to generate a score; and c) identifying the presence or absence of Sjögren's syndrome in the subject based on the score.

Embodiment 1b. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: DDX60, IFIH1, OAS3, ZC3HAV1, RSAD2, CMPK2, IFIT5, IFI6, OASL, OAS1, ISG15, MRAS, GRAMD1B, TRIM38, EPSTI1, SLC4A11, IFI16, TRIM22, RFLNB and PML; b) inputting the expression levels from step (a) into an algorithm to generate a score; and c) identifying the presence or absence of Sjögren's syndrome in the subject based on the score.

Embodiment 1c. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: DDX60, OAS3, IFI6, RSAD2, CMPK2, OAS1, OASL, ISG15, EPSTI1, USP27X, LY6E, OAS2, IFIT3, ABO, BST2, IFIT1, IFI35, SLFN5, BATF2 and DEFA1; b) inputting the expression levels from step (a) into an algorithm to generate a score; and c) identifying the presence or absence of Sjögren's syndrome in the subject based on the score.

Embodiment 1d. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: ISG15, RSAD2, TRIM38, and IFI6; b) inputting the expression levels from step (a) into an algorithm to generate a score; and c) identifying the presence or absence of Sjögren's syndrome in the subject based on the score.

Embodiment 1e. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: TP5313, NT5C3A, SAMHD1, IFITM3, XAF1, GRAMD1A, SHC2, TBC1D16, ERICH1, OTOF, APOBEC3F, SP100, GLIS2, RTP4, SERPING1, TMEM123, EIF2AK2, HERC5, LINC01473, KPTN, IFITM1, IFIT2, DDX58, SHISA5, IFI44L, IFIT1, TNFSF10, UBE2L6, USP18, BATF2, VAMP5, OAS2, GPRC5C, ZBP1, SNHG15, TOX, LY6E, IFIT3, RFLNB, MX1, PML, TRIM22, IFI16, SLC4A11, EPSTI1, MRAS, ISG15, GRAMD1B, OAS1, OASL, TRIM38, IFI6, IFIT5, RSAD2, CMPK2, ZC3HAV1, IFIH1, OAS3 and DDX60; b) inputting the expression levels from step (a) into an algorithm to generate a score; and c) identifying the presence or absence of Sjögren's syndrome in the subject based on the score.

Embodiment 1f. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: IFIH1, DDX60, OAS3 and ZC3HAV1; b) inputting the expression levels from step (a) into an algorithm to generate a score; and c) identifying the presence or absence of Sjögren's syndrome in the subject based on the score.

Embodiment 1g. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: RSAD2, IFI6, IFIT5 and CMPK2; b) inputting the expression levels from step (a) into an algorithm to generate a score; and c) identifying the presence or absence of Sjögren's syndrome in the subject based on the score.

Embodiment 1h. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: DDX60, OAS3, IFI6 and RSAD2; b) inputting the expression levels from step (a) into an algorithm to generate a score; and c) identifying the presence or absence of Sjögren's syndrome in the subject based on the score.

Embodiment 1i. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: CMPK2, OAS1, OASL and ISG15; b) inputting the expression levels from step (a) into an algorithm to generate a score; and c) identifying the presence or absence of Sjögren's syndrome in the subject based on the score.

Embodiment 1j. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: IFIH1, ISG15, EPSTI1, IFI16, RSAD2 and OAS1; b) inputting the expression levels from step (a) into an algorithm to generate a score; and c) identifying the presence or absence of Sjögren's syndrome in the subject based on the score.

Embodiment 1k. A method of identifying if a subject has Systemic Lupus Erythematosus (SLE) or Sjögren's syndrome, the method comprising: a) determining the expression of at least one biomarker selected from at least one biomarker signature in microvesicular RNA isolated from a saliva sample from the subject, wherein the at least one biomarker signature comprises the biomarkers: ISG15, IFI16, RSAD2 and OAS1; b) inputting the expression levels from step (a) into an algorithm to generate a score; and c) identifying the subject as having either SLE or Sjögren's syndrome based on the score.

Embodiment 1l. A method of identifying if a subject has Rheumatoid Arthritis (RA) or Sjögren's syndrome, the method comprising: a) determining the expression of at least one biomarker selected from at least one biomarker signature in microvesicular RNA isolated from a saliva sample from the subject, wherein the at least one biomarker signature comprises the biomarkers: IFIH1, ISG15, EPSTI1 and IFI16; b) inputting the expression levels from step (a) into an algorithm to generate a score; and c) identifying the subject as having either RA or Sjögren's syndrome based on the score.

Embodiment 1m. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: ISG15, RSAD2, IFI16 and OAS1; b) inputting the expression levels from step (a) into an algorithm to generate a score; and c) identifying the presence or absence of Sjögren's syndrome in the subject based on the score.

Embodiment 1n. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: SERPING1, RTP4, SLC4A11 and MRAS; b) inputting the expression levels from step (a) into an algorithm to generate a score; and c) identifying the presence or absence of Sjögren's syndrome in the subject based on the score.

Embodiment 1o. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: ISG15, IFIH1, EPSTI1 and IFI16; b) inputting the expression levels from step (a) into an algorithm to generate a score; and c) identifying the presence or absence of Sjögren's syndrome in the subject based on the score.

Embodiment 1p. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: NT5C3A, IFIH1, RTP4 and IFI44L; b) inputting the expression levels from step (a) into an algorithm to generate a score; and c) identifying the presence or absence of Sjögren's syndrome in the subject based on the score.

Embodiment 1q. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: ISG15, IFIH1, IFI16 and SLC4A11; b) inputting the expression levels from step (a) into an algorithm to generate a score; and c) identifying the presence or absence of Sjögren's syndrome in the subject based on the score.

Embodiment 1r. A method of identifying the presence or absence of Sjögren's syndrome in a subject, the method comprising: a) determining the expression of at least one biomarker selected from at least one biomarker signature in microvesicular RNA isolated from a saliva sample from the subject, wherein the at least one biomarker signature comprises the biomarkers: AQP1, AQP3, AQP4, AQP4-AS1, AQP5 and AQP7; b) comparing the expression levels from step (a) to corresponding predetermined cutoff values; and c) identifying the presence Sjögren's syndrome in the subject when the expression levels from step (a) are less than or equal to the corresponding predetermined cutoff values or identifying the absence of Sjögren's syndrome in the subject when the expression levels from step (a) are greater than the corresponding predetermined cutoff values.

Embodiment 1s. A method of identifying the presence or absence of Sjögren's syndrome in a subject, the method comprising: a) determining the expression of AQP9 in microvesicular RNA isolated from a saliva sample from the subject; b) comparing the expression level from step (a) to a corresponding predetermined cutoff value; and c) identifying the presence Sjögren's syndrome in the subject when the expression level from step (a) is greater than or equal to the corresponding predetermined cutoff value or identifying the absence of Sjögren's syndrome in the subject when the expression levels from step (a) is less than the corresponding predetermined cutoff value.

Embodiment 1t. A method of identifying the presence or absence of Sjögren's syndrome in a subject, the method comprising: a) determining the expression of at least one biomarker selected from at least one biomarker signature, wherein the at least one biomarker signature is a biomarker signature related to response to interferons; b) inputting the expression levels from step (a) into an algorithm to generate a score; and c) identifying the presence or absence of Sjögren's syndrome in the subject based on the score.

Embodiment 2. The method of claim embodiment 1t, wherein the biomarker signature related to response to interferons comprises the biomarkers: ISG15, IFI6, RXRA, IFIT1, STAT1, APOBEC3G, SP110, ERG, MORC3, IFI44L, MX1, SP100, LY6E, IFI44, ADAR, OAS1, IRF9, IFIT3, EIF2AK2, TGIF1, BST2, OAS2, CMTR1, UBE2L6, BRD3, IFI35 and IFI30.

Embodiment 3. The method of embodiment 1t, wherein the biomarker signature related to response to interferons is a biomarker signature related to response to interferon alpha.

Embodiment 4. The method of embodiment 3, wherein the biomarker signature related to response to interferon alpha comprises the biomarkers: IFIH1, MX1, GBP1, TNFSF10, OAS1, IFIT1, IFIT5, IFI44L, CXCL10, IFIT3, OAS2, OASL, IFI16, STAT1, ZC3HAV1, TRIM22, RSAD2, IFITM1, IFI44, IFIT2, DDX58, DDX60, USP18, RTP4, SAMD9, HERC5, SAMD9L, CMPK2, CD274, EPSTI1 and DDX60L.

Embodiment 5. The method of embodiment 1t, wherein the biomarker signature related to response to interferons is a biomarker signature related to response to interferon beta.

Embodiment 6. The method of embodiment 5, wherein the biomarker signature related to response to interferon beta comprises the biomarkers: PDZK1IP1, MYL9, SPON2, IFI44, IFI44L, LILRA3, CHI3L1, CMTM5, CLU, CMPK2, CMTM2, DTX3L, TYMP, EGR1, EGR2, APOBEC3A, SAMD9L, FCER1A, DDX58, IFIT5, IFI6, STAP1, GBP1, GGTA1, LAMP3, GP9, TRBV27, FFAR2, GZMB, TREML1, IFI27, IFI35, IFIT2, IFIT1, IFIT3, CXCL8, CXCL10, IRF7, ITGA2B, JUP, KCNJ15, KLRD1, ARG1, IFITM3P7, LGALS3BP, CYP4F3, LY6E, MMP9, MX1, MX2, OAS1, OAS2, OAS3, GOS2, LAP3, HERC5, MS4A4A, PLSCR1, XAF1, SAMD9, HERC6, TMEM140, DDX60, EIF2AK2, PROS1, CABP5, HES4, RPS23, CCL2, CCL8, RTP4, MMP25, PARP12, SIGLEC1, STAT1, SERPING1, C1QA, C1QB, TNFAIP6, C1QC, C3AR1, DHX58, ZBP1, TCL1A, PARP9, SH3BGRL2, OASL, PNPT1, MGAM, RSAD2, CD163, EPSTI1, APOBEC3B, ISG15 and SCO2.

Embodiment 7. The method of any one of the preceding embodiments, wherein step (a) comprises determining the expression level of each of the biomarkers in the biomarker signature.

Embodiment 8. The method of any one of the preceding embodiments, wherein identifying the presence or absence of Sjögren's syndrome in the subject based on the score comprises: i) comparing the score to a predetermined cutoff value; and ii) determining that the at the subject has Sjögren's syndrome when the score is greater than or equal to the predetermined cutoff value or determining that the subject does not have Sjögren's syndrome when the score is less than the predetermined cutoff value.

Embodiment 9. The method of any one of the preceding embodiments, wherein the algorithm is the product of a feature selection wrapper algorithm, a machine learning algorithm, a trained classifier built from at least one predictive classification algorithm or any combination thereof.

Embodiment 10. The method of embodiment 9, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

Embodiment 11. The method of embodiment 9 or 10, wherein the algorithm is the product of a trained classifier is trained to identify Sjögren's syndrome in a subject using: i) the expression level of the at least one biomarker in at least one biological sample from at least one subject who does not have Sjögren's syndrome; and ii) the expression levels of the at least one biomarker in at least one biological sample from at least one subject who has Sjögren's syndrome.

Embodiment 12. The method of any of the preceding embodiments, wherein the sample is collected using sample home-collection device.

Embodiment 13. The method of any of the preceding embodiments, further comprising prior to step (a): i) isolating a plurality of microvesicles from the saliva sample from the subject; and ii) extracting microvesicular RNA from the plurality of isolated microvesicles.

Embodiment 14. The method of embodiment 12, wherein prior to step (i), at least one stabilizing agent is added to the saliva sample, preferably wherein the at least one stabilizing agent is an RNAse inhibitor.

Embodiment 15. The method of any of the preceding embodiments, wherein the saliva samples are filtered, preferably wherein the saliva samples are filtered using a filter with an average pore size of about 0.8 μm.

Embodiment 16. The method of anyone of embodiments 12-15, further comprising fragmenting the extracted microvesicular RNA.

Embodiment 17. The method of any one of embodiments 12-16, further comprising contacting the extracted microvesicular RNA with Solid-phase reversible immobilization (SPRI) beads.

Embodiment 18. The method of any one of embodiments 12-17, wherein the extracted microvesicular RNA is amplified using PCR, preferably wherein the amplification is performed for about 18 cycles.

Embodiment 19. The method of any of the preceding embodiments, wherein the at least one microvesicle is isolated from the saliva sample by contacting the saliva sample with at least one affinity agent that binds to at least one surface marker present on the surface the at least one microvesicle.

Embodiment 20. The method of any of the preceding embodiments, wherein step (a) further comprises: (i) determining the expression level of at least one reference biomarker; (ii) normalizing the expression level of the at least one biomarker to the expression level of the at least one reference biomarker.

Embodiment 21. The method of any of the preceding embodiments, wherein inputting the expression levels from step (a) into an algorithm to generate a score comprises inputting the normalized expression levels from step (a) into an algorithm to generate a score.

Embodiment 22. The method of any of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

Embodiment 23. The method of embodiment 22, wherein determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

Embodiment 24. The method of any of the preceding embodiments, wherein the predetermined cutoff value has a negative predictive value of at least about 80%, or at least about 85%, or at least about 90%, or at least about 95%, or at least about 99%, or at least about 99.9%.

Embodiment 25. The method of any of the preceding embodiments, wherein the predetermined cutoff value has a positive predictive value of at least about 80%, or at least about 85%, or at least about 90%, or at least about 95%, or at least about 99%, or at least about 99.9%.

Embodiment 26. The method of any of the preceding embodiments, wherein the predetermined cutoff value has a sensitivity of at least about 80%, or at least about 85%, or at least about 90%, or at least about 95%, or at least about 99%, or at least about 99.9%.

Embodiment 27. The method of any of the preceding embodiments, wherein the predetermined cutoff value has a specificity of at least about 80%, or at least about 85%, or at least about 90%, or at least about 95%, or at least about 99%, or at least about 99.9%.

Embodiment 28. The method of any of the preceding embodiments, wherein the predetermined cutoff value is calculated using at least one receiver operating characteristic (ROC) curve.

Embodiment 29. The method of any of the preceding embodiments, wherein measuring expression levels in step (a) further comprises selectively enriching for the at least one biomarker.

Embodiment 30. The method of any of the preceding embodiments, wherein the at least one biomarker is selectively enriched by hybrid-capture, preferably wherein: i) the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome; and/or ii) the hybrid-capture results in a significant depletion in microbial nucleic acids

Embodiment 31. The method of any of the preceding embodiments, further comprising administering at least one treatment to a subject identified as having Sjögren's syndrome.

Embodiment 32. The method of embodiment 31, wherein the at least one treatment comprises: i) administering at least one therapeutically effective amount of an cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof; ii) surgery, preferably wherein the surgery comprises sealing the tear ducts of the subject; or iii) a combination thereof.

### EXAMPLES

### Example 1-Cohort 1

Saliva samples were collected from 11 subjects with Sjögren's syndrome, 10 subjects who did not have Sjögren's syndrome (“healthy matched controls”), five subjects with rheumatoid arthritis (RA) and five subjects who have systemic lupus erythematosus (SLE).

Microvesicles were isolated from the saliva samples using the methods of the present disclosure. Microvesicular RNA was then extracted from the isolated microvesicles and the RNA was analyzed using next-generation sequencing following the preparation of a sequencing library from the extracted microvesicular RNA. As part of the next-generation sequencing analysis, hybrid-capture was used to enrich for human exome transcripts and long intervening/intergenic noncoding RNAs (lincRNAs). ERCC RNA spike-in mix was also used as a control. Hybrid-capture was also used to enrich for human transcripts of any size of a defined panel of genes consisting of at least two genes, and in any combination with lincRNA and ERCC RNA as a control.

Specific alternatives within the microvesicle isolation/microvesicular RNA extraction workflow were tested to determine their effect on overall quality of the final next-generation sequencing analysis.

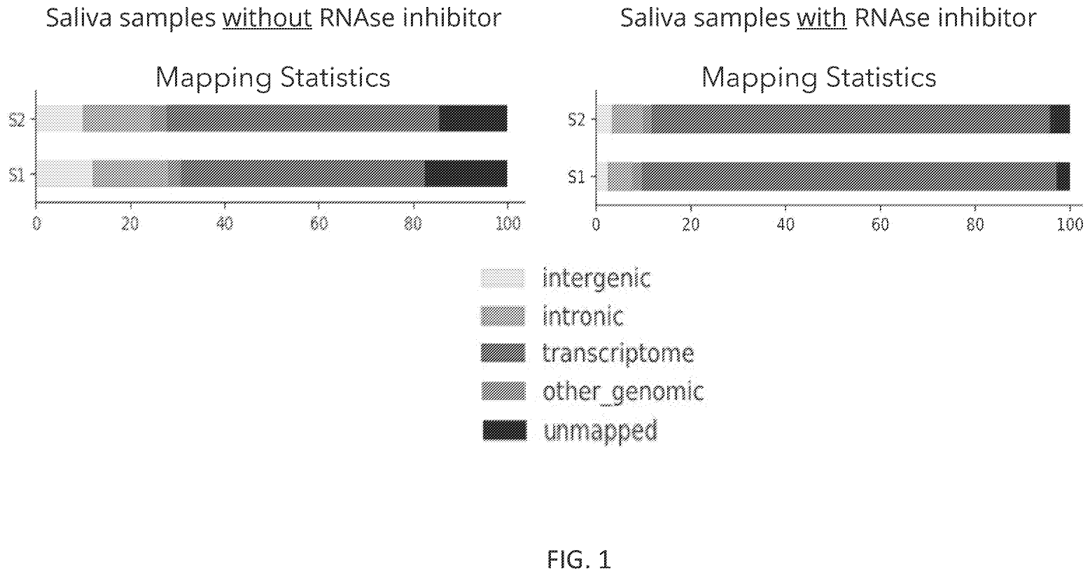

First, saliva samples were processed and analyzed with or without the addition of an RNAse inhibitor to the saliva sample. FIG. 1 shows the mapping statistics (i.e. the percentage of final sequencing reads that are mapped to either intergenic nucleic acid, intronic nucleic acid, transcriptome nucleic acid, other genomic nucleic acid, or nucleic acid that is unmappable) obtained from saliva samples that were analyzed without the addition of an RNase inhibitor (left panel) and from saliva samples that were analyzed with the addition of an RNAse inhibitor. As would be appreciated by the skilled artisan, increases in the number of reads mapped to transcriptome nucleic acid represents a final analysis of higher quality. As shown in FIG. 1, the addition of RNAse inhibitor resulted in approximately a 30% increase in the percentage of final sequencing reads that are mapped to transcriptome nucleic acid.

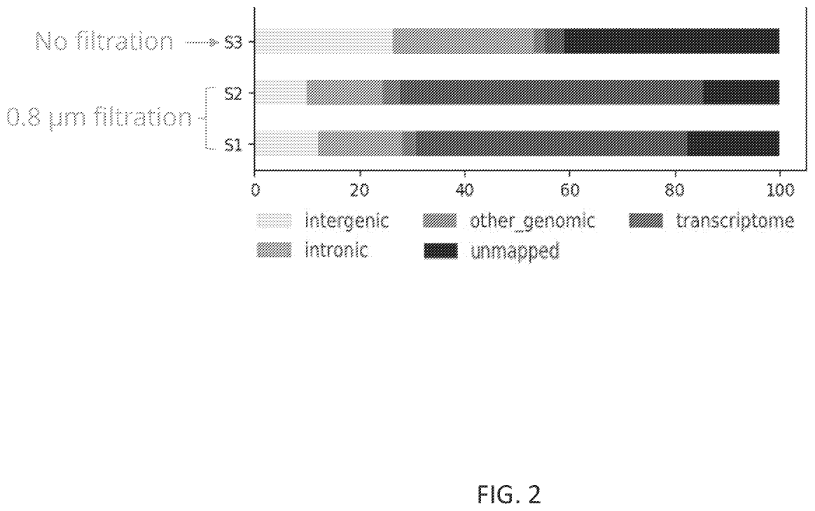

Saliva samples were also processed and analyzed with or without filtering of the saliva sample. FIG. 2 shows the mapping statistics of final sequencing reads obtained from saliva samples that were analyzed without filtering and from saliva samples that were analyzed with filtering. As shown in FIG. 2, filtering the saliva samples resulted in an approximately 10× increase in the percentage of final sequencing reads that were mapped to transcriptome nucleic acid.

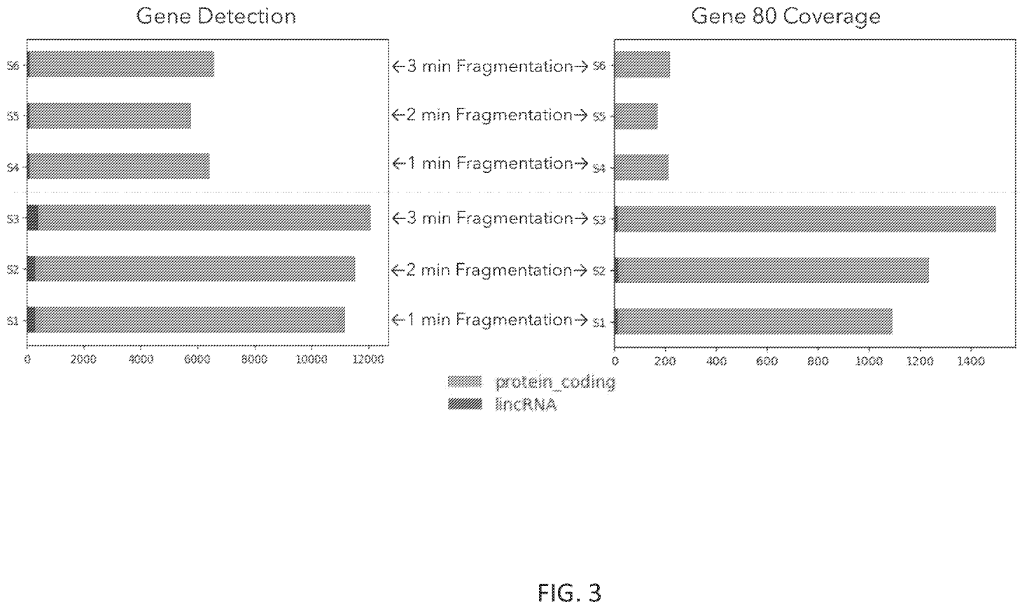

Extracted microvesicular RNA was also subjected to fragmentation for either 1 minute, 2 minutes or 3 minutes at 85° C. FIG. 3 shows the number of genes detected (left panel) and the Gene 80 coverage (right panel) for the varying fragmentation times.

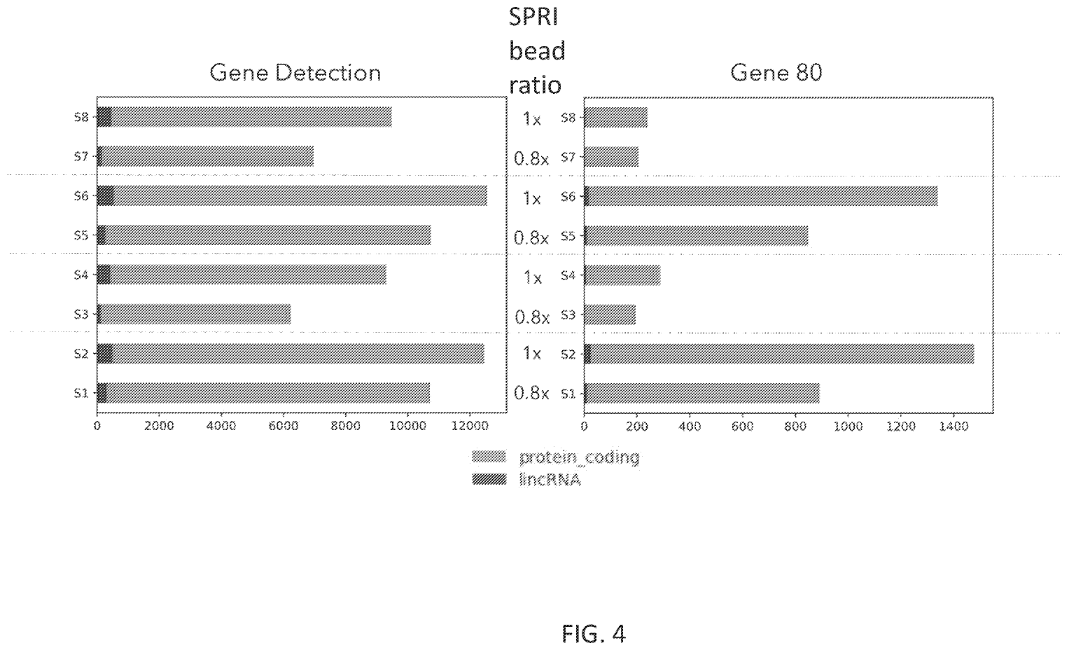

Extracted microvesicular RNA was also further purified using solid-phase reversible immobilization (SPRI) beads at varying ratios of beads to sample. FIG. 4 shows the number of genes detected (left panel) and the Gene 80 coverage (right panel) for the varying SPRI bead ratios tested.

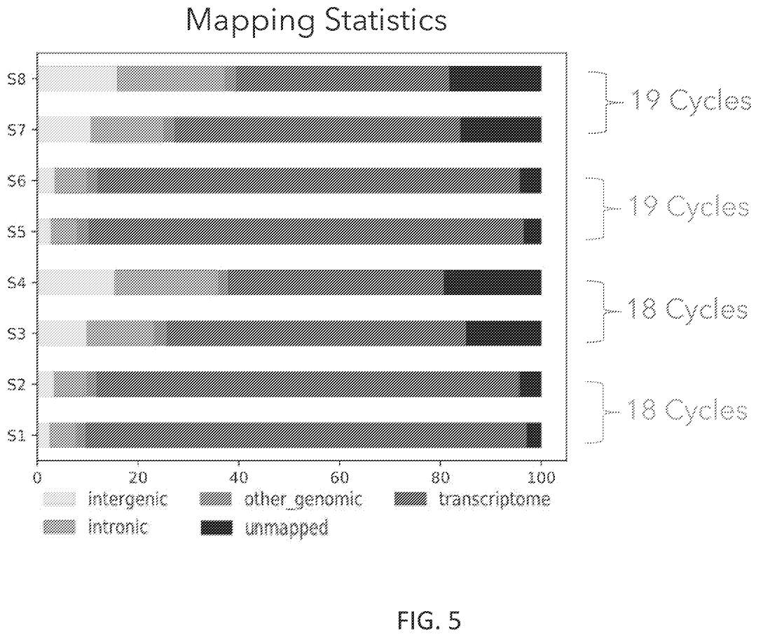

The number of final PCR amplification cycles in the library construction step of the analysis was also varied to determine its effect on the quality of the final sequencing analysis. FIG. 5 shows the mapping statistics for libraries that were produced with a final PCR amplification of 19 cycles or 18 cycles.

For the final analyses described below, the saliva samples were treated with RNAse inhibitor and filtered, a three-minute fragmentation at 85° C. was used, an SPRI bead purification ratio of 1:1 was used, and 18 cycles of final PCR amplification was used.

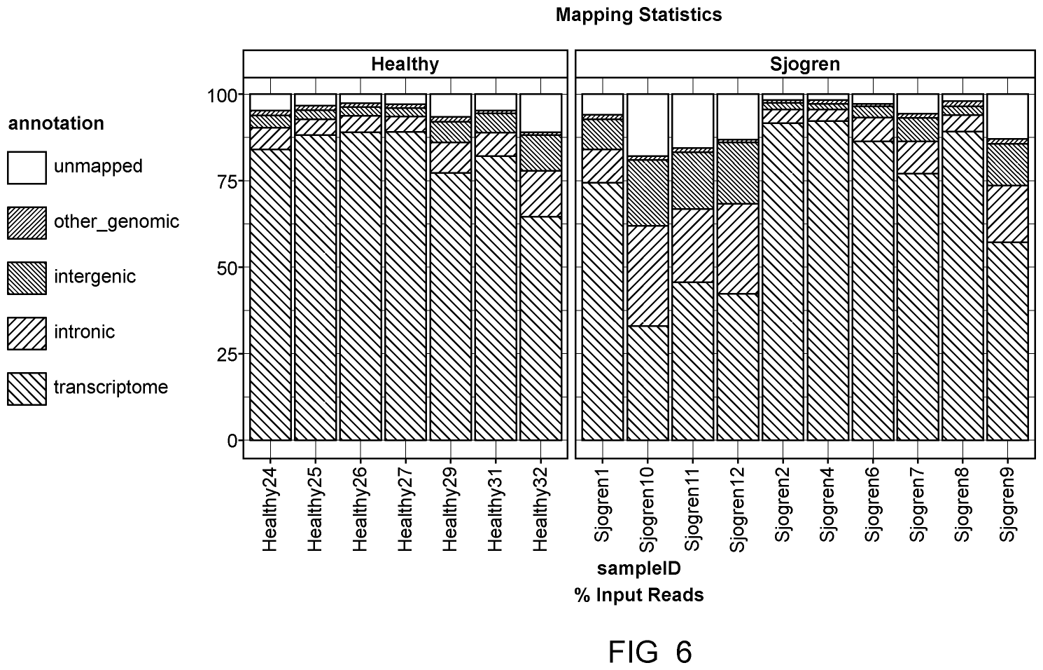

Sequencing reads were aligned to the human genome (GRCh38) with Spliced Transcripts Alignment to a Reference (STAR) software. Gene level expression was quantified from the mapped reads with Salmon using gencode.v25 gene annotations. FIG. 6 shows the mapping statistics for the final sequencing reads obtained in the analysis of the Sjögren's syndrome saliva samples and various healthy matched control samples.

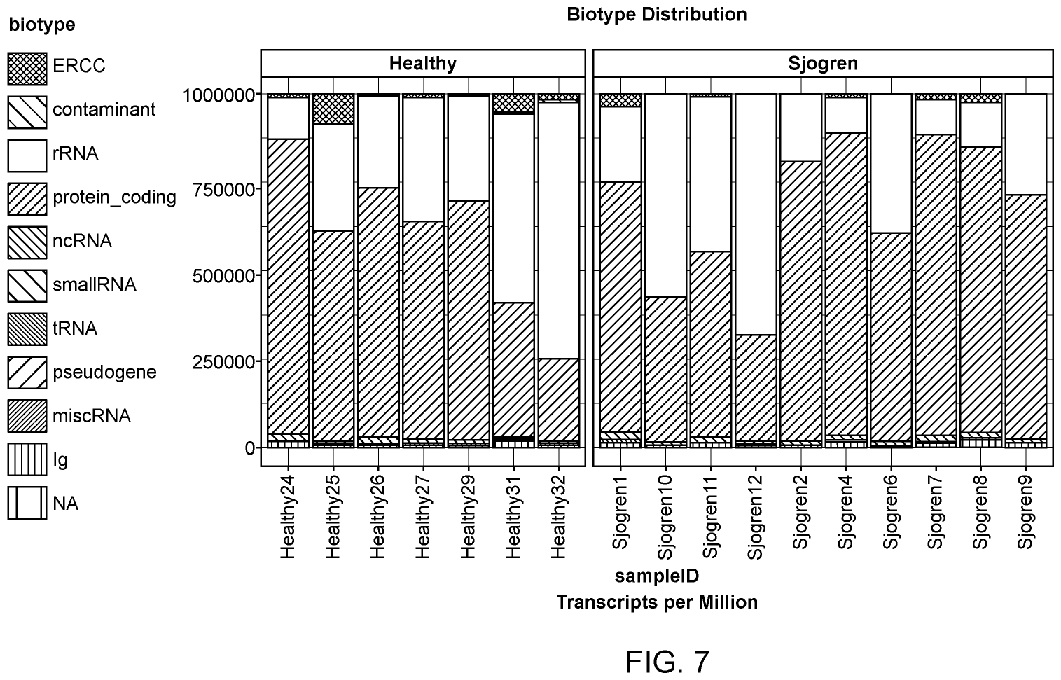

FIG. 7 shows the biotype distribution for the final sequencing reads obtained in the analysis of the Sjögren's syndrome saliva samples and various healthy matched control saliva samples.

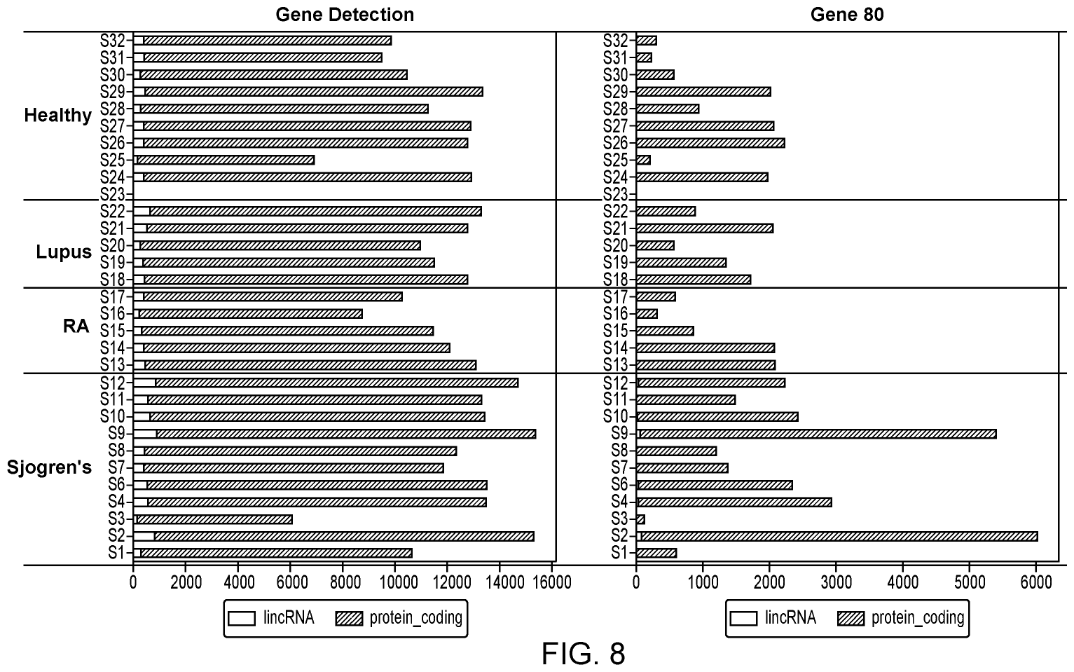

FIG. 8 shows the number of genes detected (left panel) and the Gene 80 coverage (right panel) in the final sequencing analysis for the Sjögren's syndrome saliva samples, the healthy matched control saliva samples, the RA saliva samples and the SLE saliva samples.

Only samples with at least ten million sequencing reads passed the quality control inclusion criteria for downstream analysis. Differential Expression analysis was then performed on the sequencing data obtained from the Sjögren's syndrome saliva samples, the healthy matched control saliva samples, the RA saliva samples and the SLE saliva samples.

Table 1 shows the number of differentially expressed genes between the healthy samples and the specific disease samples (i.e., either Sjögren's syndrome, RA or SLE samples), the number of upregulated genes in the disease samples, and the number of upregulated genes in the healthy control matched samples.

TABLE 1

| | Differentially | Genes | Genes |
|---|---|---|---|
| Disease vs | Expressed | Upregulated | Upregulated in |
| Healthy | Genes | in Disease | Healthy |
| Sjögren's | 70 | 64 | 6 |
| Syndrome | | | |
| RA | 8 | 2 | 6 |
| SLE | 9 | 5 | 4 |

Table 2 shows the number of differentially expressed genes between the Sjögren's syndrome samples and either the RA samples or the SLE samples specific disease samples, the number of upregulated genes in the RA or the SLE samples, and the number of upregulated genes in the Sjögren's syndrome samples.

TABLE 2

| | Differentially | Genes | |
|---|---|---|---|
| Sjögren's vs | Expressed | Upregulated | Genes Upregulated |
| Disease | Genes | in Disease | in Sjögren's |
| RA | 105 | 19 | 85 |
| SLE | 4 | 1 | 3 |

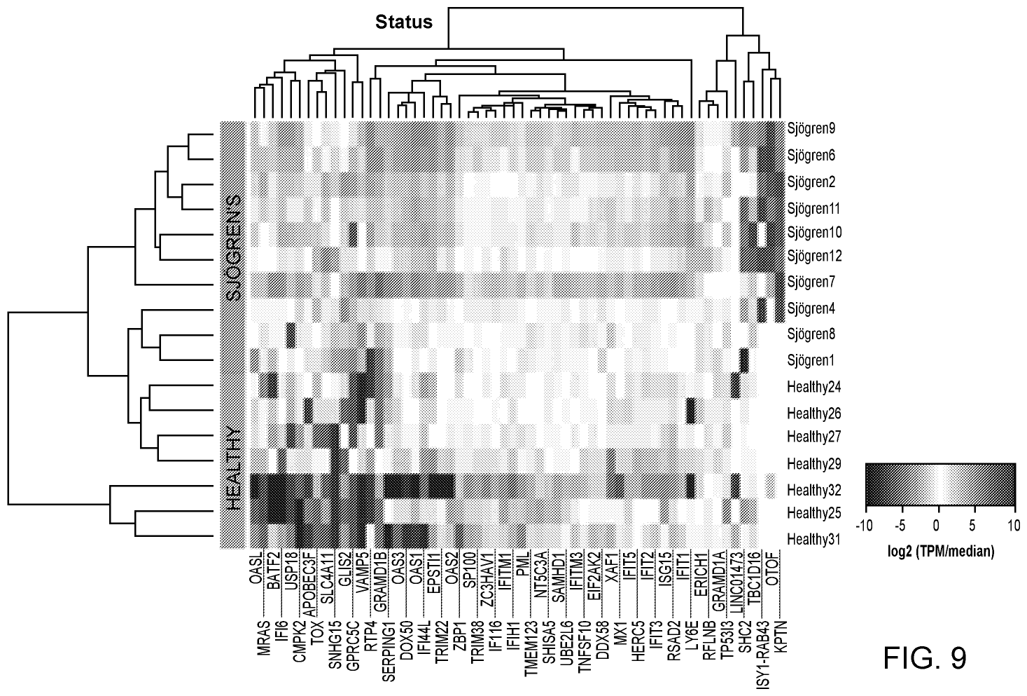

FIG. 9 shows a heatmap of genes that are differentially expressed between healthy control saliva samples and Sjögren's syndrome saliva samples.

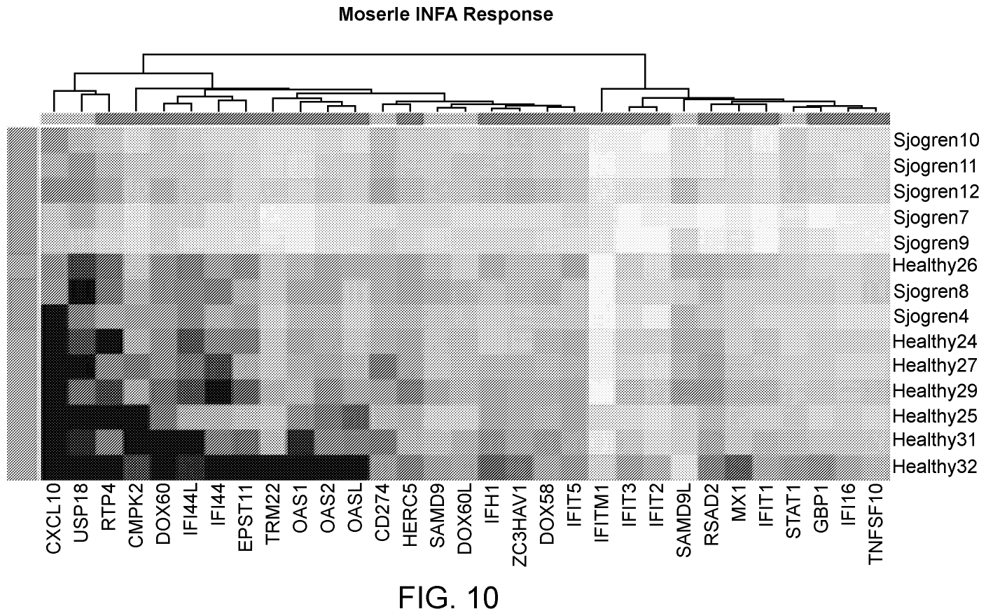

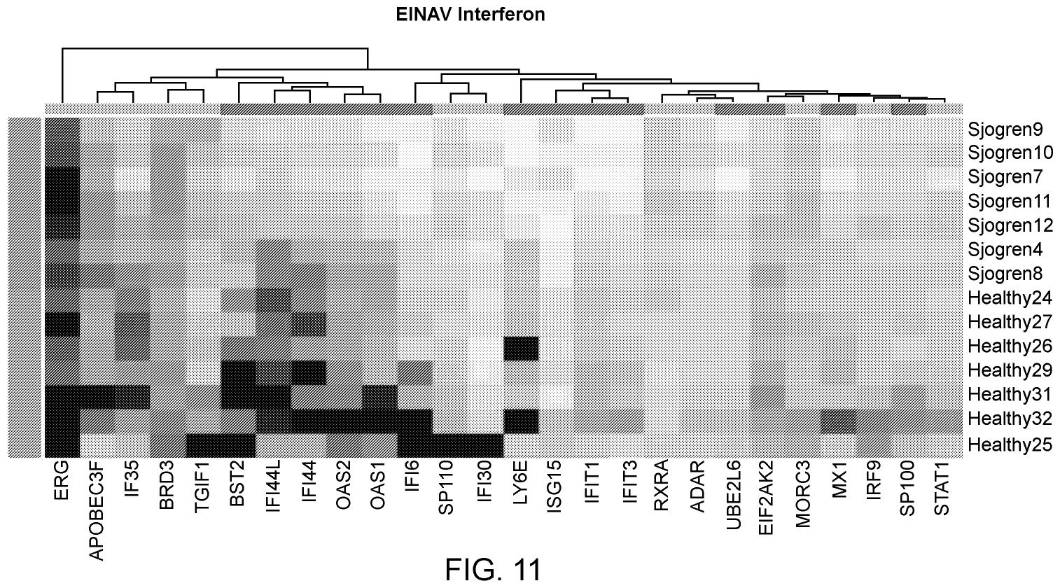

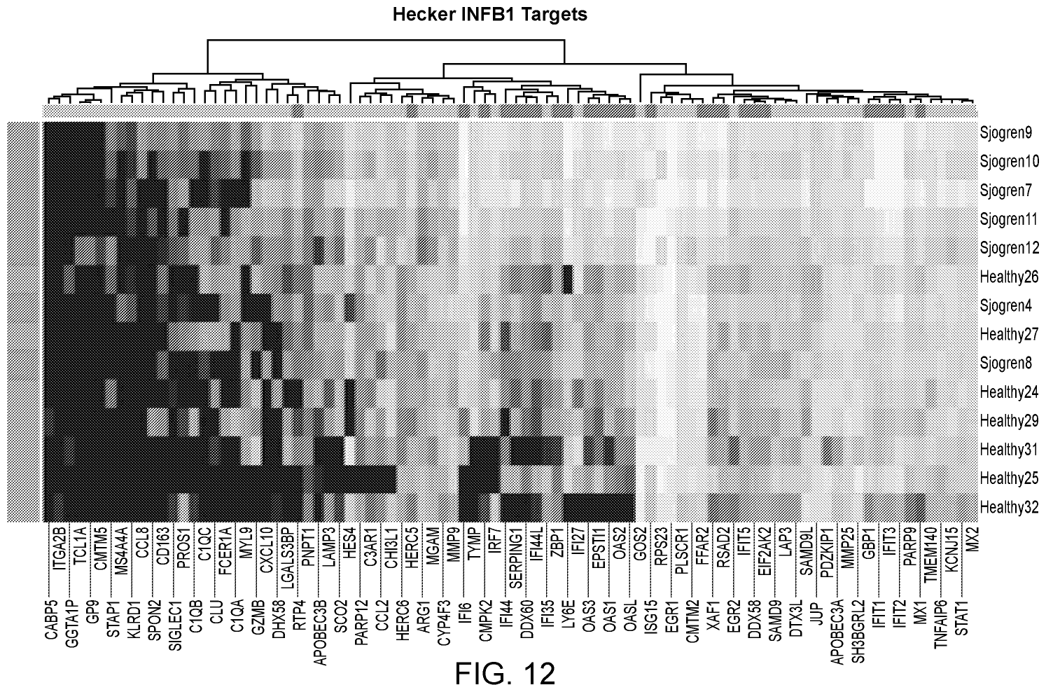

FIG. 10-12 is a series of heat maps of genes that are implicated in the interferon alpha and interferon beta response pathways that are differentially expressed between healthy control saliva samples and Sjögren's syndrome saliva samples.

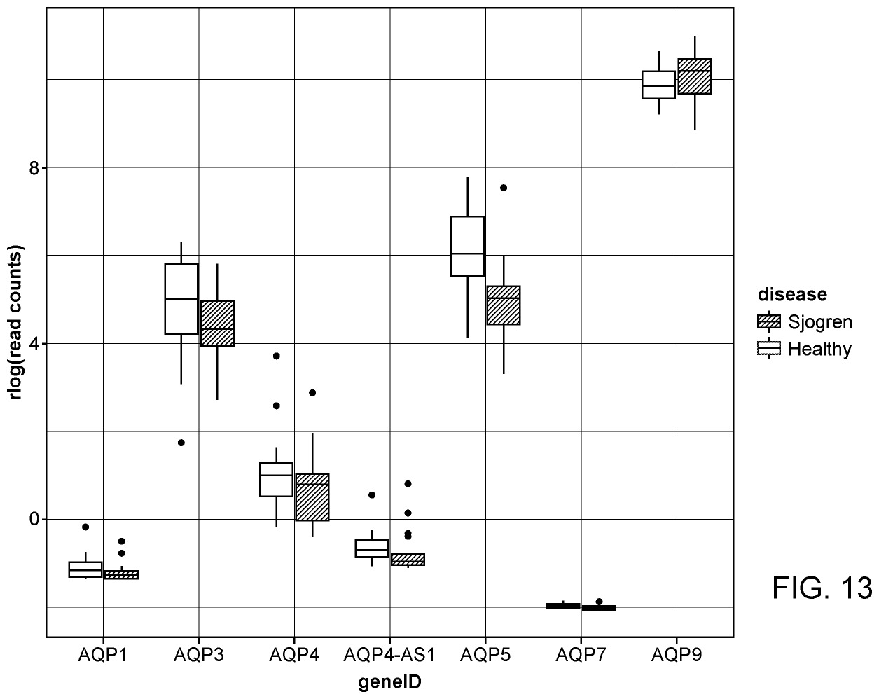

FIG. 13 is a graph showing the expression of various aquaporin genes in healthy control saliva samples (left box plots in each group) and Sjögren's syndrome saliva samples (right box plot in each group).

The differential expression analysis described above was then used to derive different biomarker signatures to differentiate between saliva samples that are from a subject having Sjögren's syndrome and from a subject that is healthy. These biomarker signatures were then tested on sequencing results obtained from a different set of saliva samples obtained from subjects with Sjögren's syndrome (n=7) and healthy (n=10) (the “test set”).

Feature selection using Boruta was performed on the differentially expressed genes. Table 3 shows the top 20 feature selected genes chosen from the genes that are differentially expressed in Sjögren's syndrome samples vs healthy samples. Table 4 shows the top 20 feature selected genes with the highest variance between Sjögren's syndrome samples vs healthy samples.

TABLE 3

| | | log2 Fold | | |
|---|---|---|---|---|
| | | Change | | |
| | | (Sjögren's | | |
| | | syndrome vs | | |
| | | healthy; | | |
| | | positive | | |
| | | values are | | |
| | | upregulated | | |
| | | in Sjögren's | | |
| | | syndrome | | |
| | Mean | compared to | Adjusted | |
| Gene ID | Importance | healthy) | p-value | Gene Type |
| DDX60 | 4.992905482 | 4.0914134 | 7.35E−05 | protein_coding |
| IFIH1 | 4.986984896 | 2.0966794 | 0.00138185 | protein_coding |
| OAS3 | 4.843020053 | 4.5740925 | 9.75E−08 | protein_coding |
| ZC3HAV1 | 4.834549054 | 1.66319 | 0.0234422 | protein_coding |
| RSAD2 | 3.593170676 | 4.7561277 | 4.46E−12 | protein_coding |
| CMPK2 | 3.545392077 | 3.7506837 | 0.00929672 | protein_coding |
| IFIT5 | 3.537024069 | 2.8264963 | 1.67E−06 | protein_coding |
| IFI6 | 3.499555691 | 4.7481125 | 4.81E−05 | protein_coding |
| OASL | 3.131214259 | 3.7553271 | 7.35E−05 | protein_coding |
| OAS1 | 3.060352749 | 4.7818835 | 1.02E−06 | protein_coding |
| ISG15 | 3.025524022 | 3.6499034 | 3.61E−06 | protein_coding |
| MRAS | 2.995083605 | 3.4319097 | 0.03733606 | protein_coding |
| GRAMD1B | 2.983315302 | 6.2665667 | 0.00055398 | protein_coding |
| TRIM38 | 2.970128035 | 1.7191789 | 0.00071766 | protein_coding |
| EPSTI1 | 2.929822083 | 3.8989574 | 0.00110474 | protein_coding |
| SLC4A11 | 2.444391242 | 4.9912484 | 0.00074211 | protein_coding |
| IFI16 | 2.317303475 | 1.6626067 | 0.00935569 | protein_coding |
| TRIM22 | 2.205356317 | 2.8246683 | 0.00341726 | protein_coding |
| RFLNB | 2.009191728 | −1.717199 | 0.00370067 | |
| PML | 1.980142079 | 2.2463157 | 0.00077413 | protein_coding |

TABLE 4

| | | log2 Fold | | |
|---|---|---|---|---|
| | | Change | | |
| | | (Sjögren's | | |
| | | syndrome vs | | |
| | | healthy; | | |
| | | positive | | |
| | | values are | | |
| | | upregulated | | |
| | | in Sjögren's | | |
| | | syndrome | | |
| | Mean | compared to | Adjusted | |
| Gene ID | Importance | healthy) | p-value | Gene Type |
| DDX60 | 6.23193172 | 4.091413 | 7.35E−05 | protein_coding |
| OAS3 | 6.141999717 | 4.574093 | 9.75E−08 | protein_coding |
| IFI6 | 4.634477036 | 4.748113 | 4.81E−05 | protein_coding |
| RSAD2 | 4.624079118 | 4.756128 | 4.46E−12 | protein_coding |
| CMPK2 | 4.478280895 | 3.750684 | 0.0093 | protein_coding |
| OAS1 | 4.102339304 | 4.781883 | 1.02E−06 | protein_coding |
| OASL | 4.070719514 | 3.755327 | 7.35E−05 | protein_coding |
| ISG15 | 3.945309925 | 3.649903 | 3.61E−06 | protein_coding |
| EPSTI1 | 3.77531128 | 3.898957 | 0.0011 | protein_coding |
| USP27X | 2.76995075 | | | protein_coding |
| LY6E | 2.706073977 | 4.192293 | 0.00072 | protein_coding |
| OAS2 | 2.522857492 | 4.334145 | 1.19E−07 | protein_coding |
| IFIT3 | 2.512984317 | 3.215549 | 4.34E−06 | protein_coding |
| ABO | 2.459571444 | | | protein_coding |
| BST2 | 2.192809029 | 3.603226 | 0.11029 | protein_coding |
| IFIT1 | 1.954941934 | 3.567131 | 1.97E−07 | protein_coding |
| IFI35 | 1.866418366 | 2.567945 | 0.25631 | protein_coding |
| SLFN5 | 1.657592392 | 2.948754 | 0.07376 | protein_coding |
| BATF2 | 1.460328566 | 3.815423 | 0.01408 | protein_coding |
| DEFA1 | 1.441332142 | −2.11576 | 0.94131 | protein_coding |

Single gene logistic modeling was performed on the differentially expressed genes and the area under the curve (AUC) values for each single-gene model was calculated based on the model's performance on the independent “test set”. Table 5 shows the top 19 genes based on AUC. For each combination of four single-gene models evaluated, they were also modeled as a multiple four-gene logistic regression model to demonstrate their utility as a single biomarker signature.

TABLE 5

| Gene | AUC |
|---|---|
| ISG15 | 0.979592 |
| RSAD2 | 0.959184 |
| TRIM38 | 0.959184 |
| IFI6 | 0.918367 |
| IFIH1 | 0.897959 |
| EPSTI1 | 0.877551 |
| IFI16 | 0.877551 |
| IFIT3 | 0.877551 |
| IFIT5 | 0.877551 |
| MX1 | 0.857143 |
| TRIM22 | 0.857143 |
| LY6E | 0.836735 |
| OAS3 | 0.836735 |
| ZC3HAV1 | 0.836735 |
| DDX60 | 0.816327 |
| OAS1 | 0.816327 |
| OASL | 0.816327 |
| CMPK2 | 0.795918 |
| PML | 0.795918 |

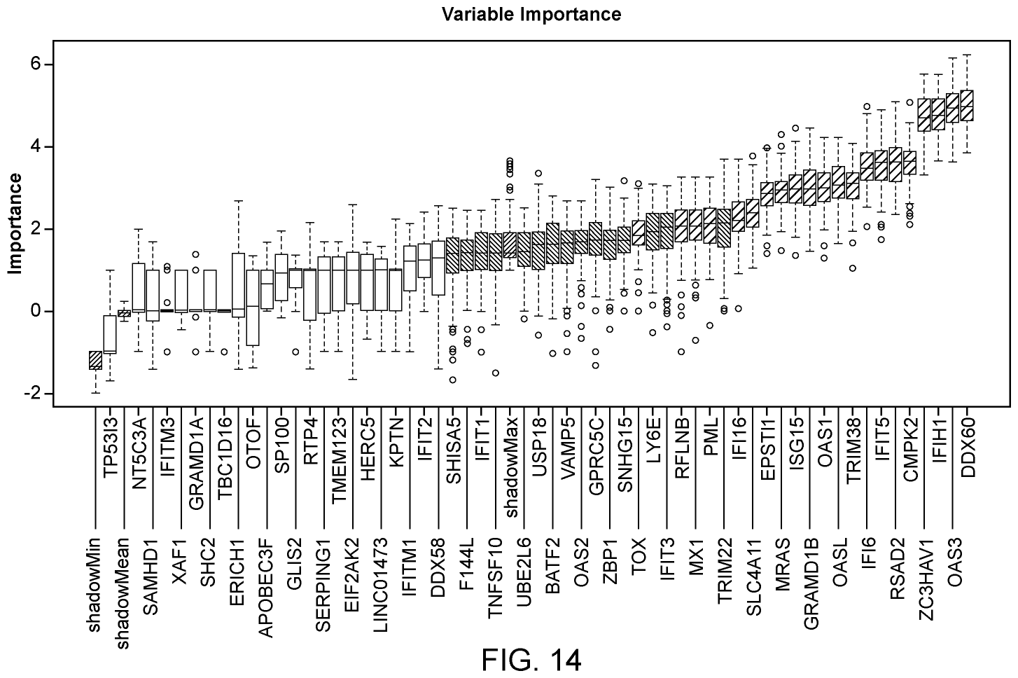

FIG. 14 shows the variable importance for various genes identified by the feature selection.

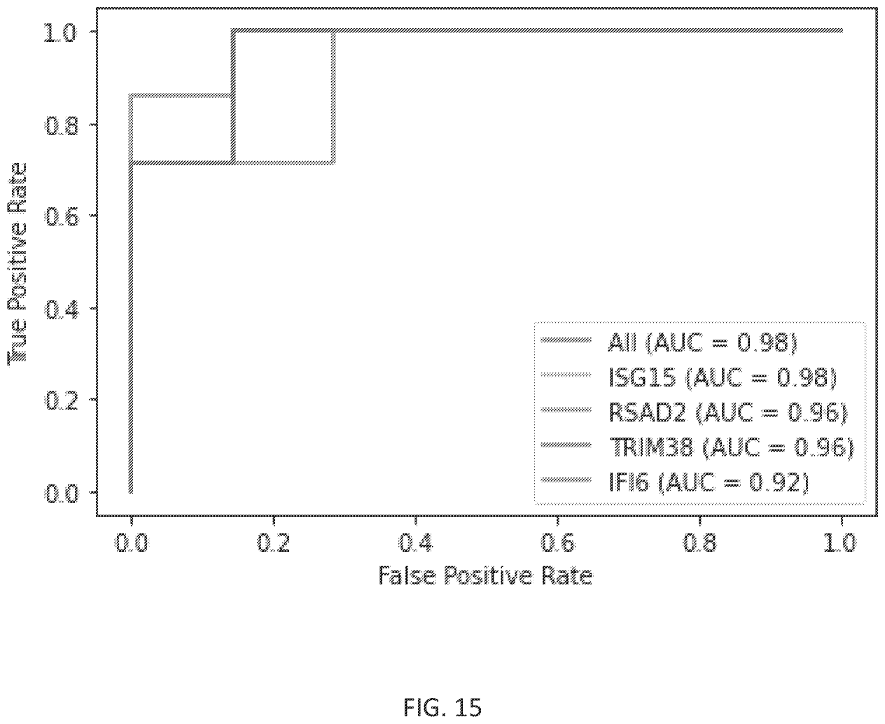

FIG. 15 shows receiver-operating characteristic (ROC) curve analysis for ISG15, RSAD2, TRIM38 and IFI6, as well as all four genes together as a single biomarker signature. The AUC for the combined four biomarker signature was 0.98, as shown in FIG. 15.

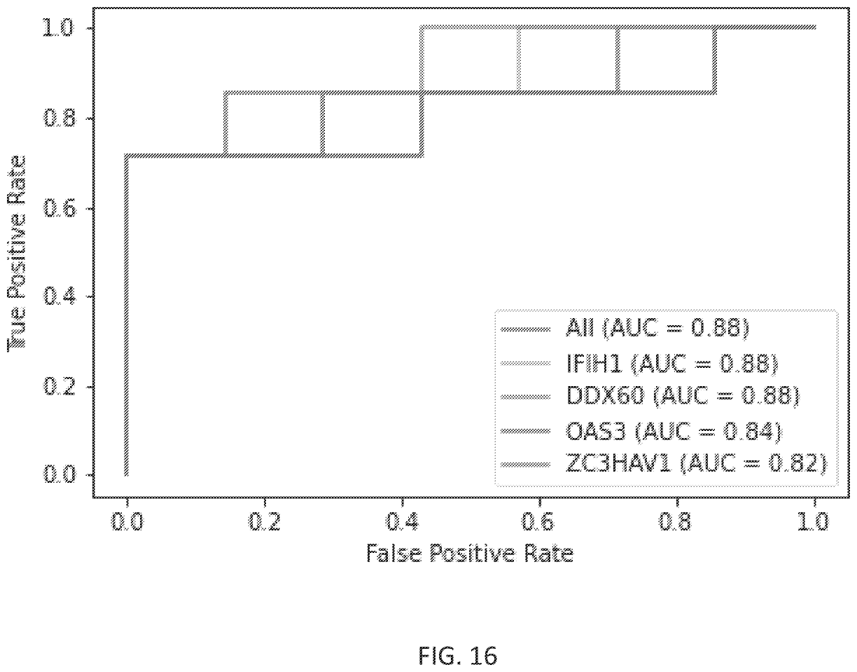

FIG. 16 shows ROC curve analysis for IFIH1, DDX60, OAS3 and ZC3HAV1, as well as all four genes together as a single biomarker signature. The AUC for the combined four biomarker signature was 0.88, as shown in FIG. 16.

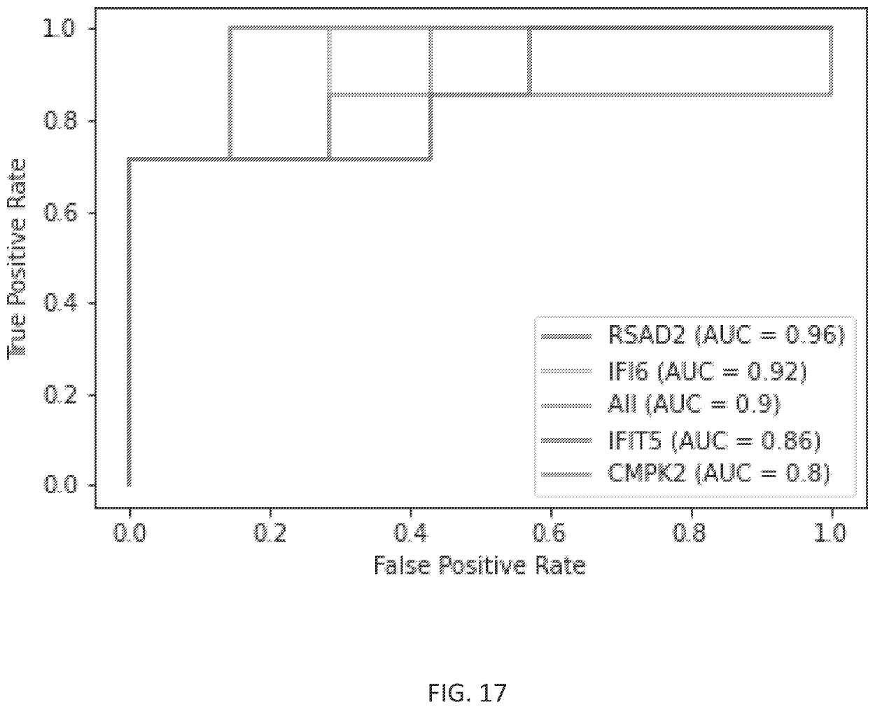

FIG. 17 shows ROC curve analysis for RSAD2, IFI6, IFIT5 and CMPK2, as well as all four genes together as a single biomarker signature. The AUC for the combined four biomarker signature was 0.9, as shown in FIG. 17.

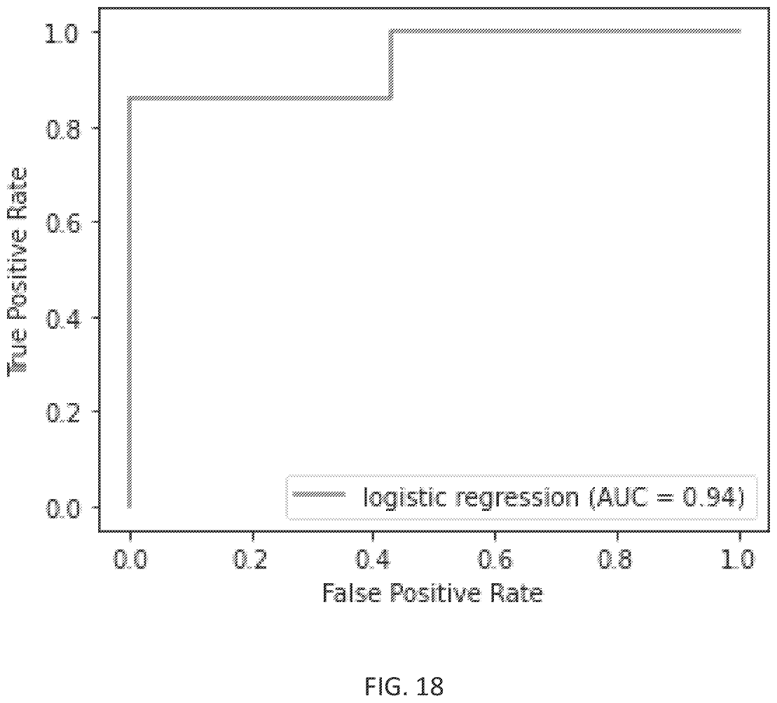

FIG. 18 shows ROC curve analysis for a biomarker signature comprising the biomarkers DDX60, OAS3, IFI6 and RSAD2. The AUC for the four-biomarker signature was 0.94, as shown in FIG. 18.

The differential expression analysis described above was also used to derive different biomarker signatures to differentiate between saliva samples that are from a subject having Sjögren's syndrome and from a subject that is healthy by selecting gene models whose scores correctly classified saliva samples from a subject having RA and/or SLE as Sjögren's syndrome negative. Thus, the biomarkers were selected based on whether or not they had an association with RA and/or SLE.

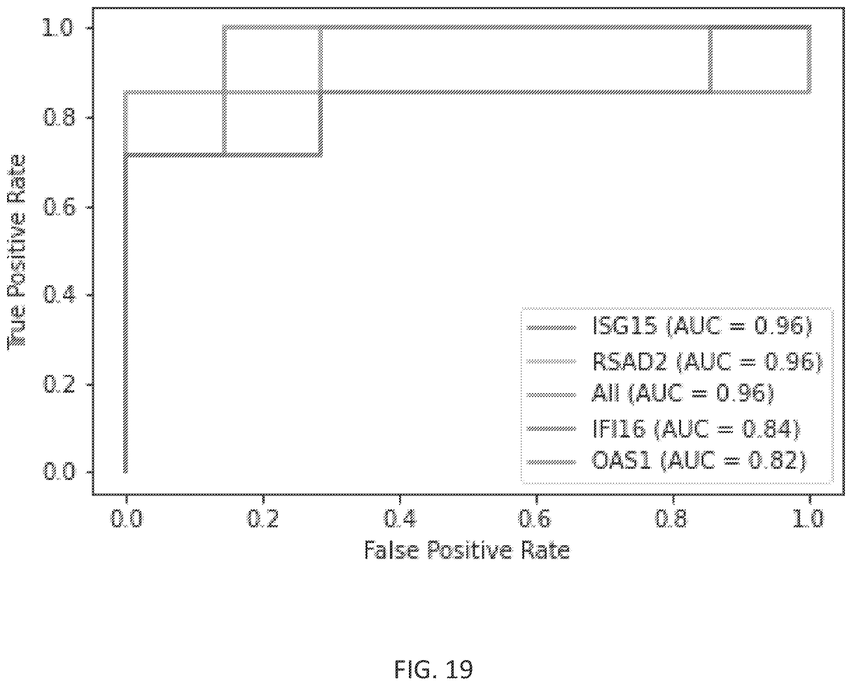

The genes ISG15, RSAD2, IFI16 and OAS1 were identified as having low association to saliva samples from subjects having SLE. FIG. 19 shows ROC curve analysis for ISG15, RSAD2, IFI16 and OAS1, as well as all four genes together as a single biomarker signature. The AUC for the combined four biomarker signature was 0.96, as shown in FIG. 19.

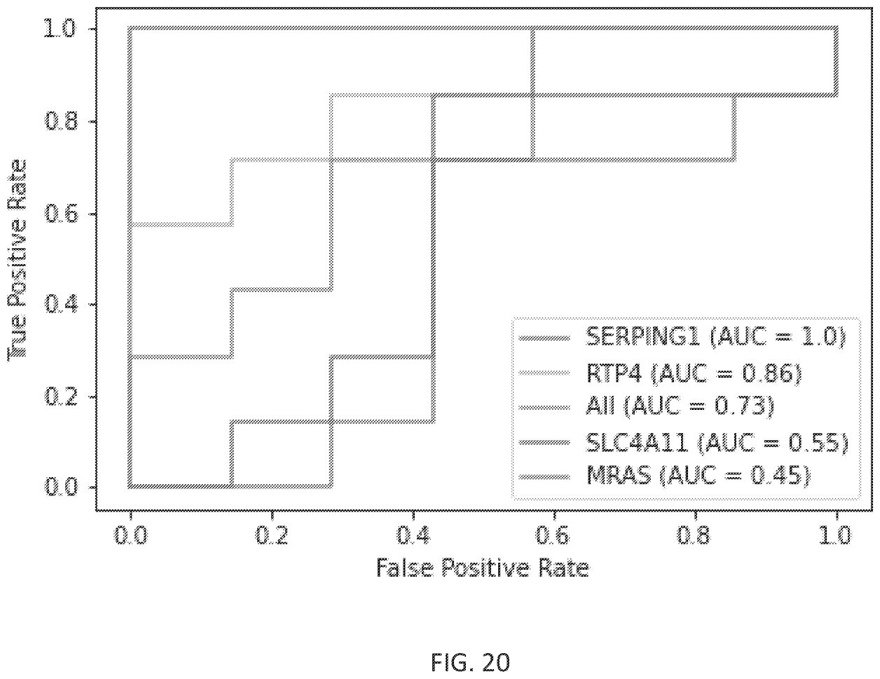

The genes SERPING1, RTP4, SLC4A11 and MRAS were also identified as having low association to saliva samples from subjects having SLE. FIG. 20 shows ROC curve analysis SERPING1, RTP4, SLC4A11 and MRAS, as well as all four genes together as a single biomarker signature. The AUC for the combined four biomarker signature was 0.73, as shown in FIG. 20.

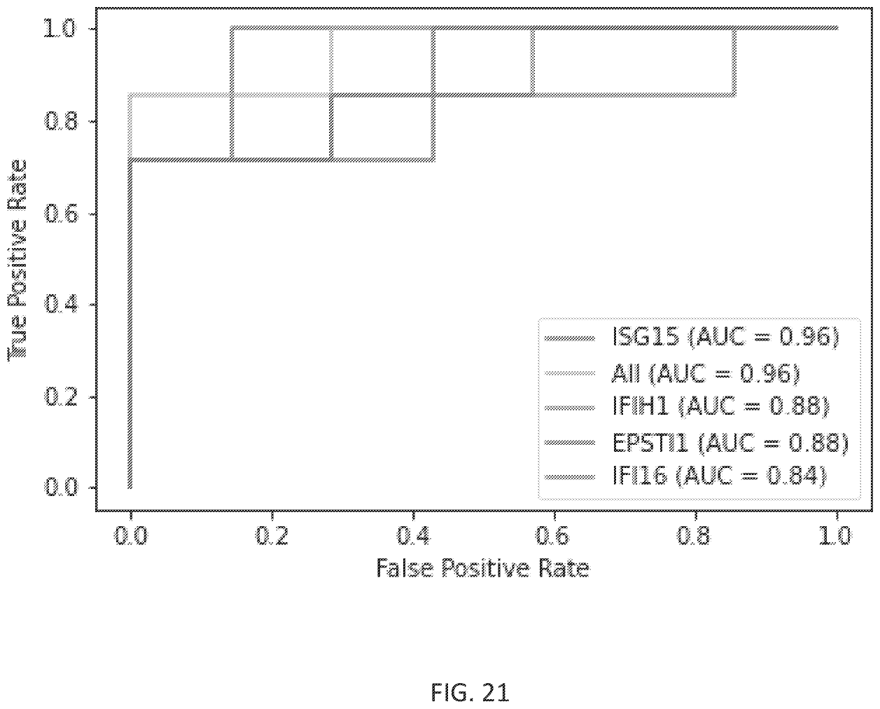

The genes ISG15, IFIH1, EPSTI1 and IFI16 were identified as having low association to saliva samples from subjects having RA. FIG. 21 shows ROC curve analysis for ISG15, IFIH1, EPSTI1 and IFI16, as well as all four genes together as a single biomarker signature. The AUC for the combined four biomarker signature was 0.96, as shown in FIG. 21.

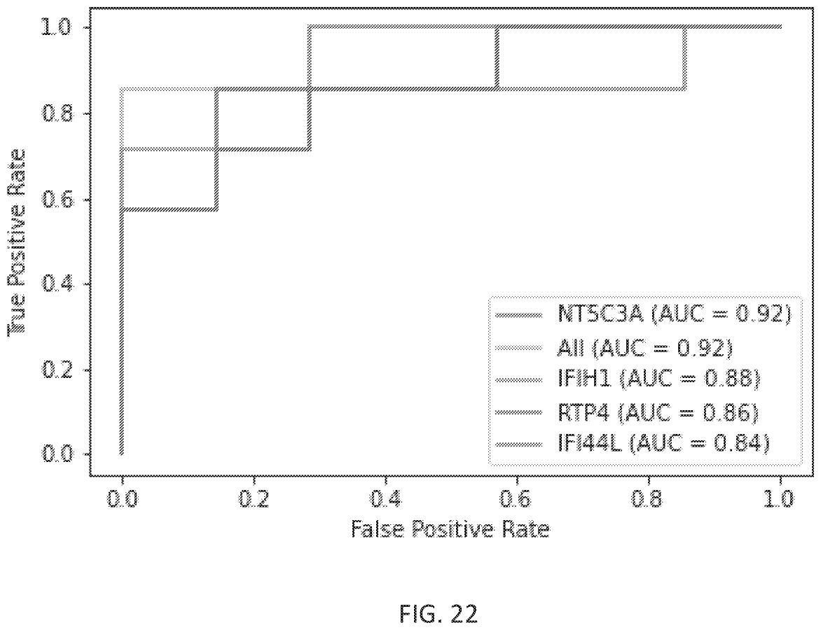

The genes NT5C3A, IFIH1, RTP4 and IFI44L were also identified as having low association to saliva samples from subjects having RA. FIG. 22 shows ROC curve analysis NT5C3A, IFIH1, RTP4 and IFI44L, as well as all four genes together as a single biomarker signature. The AUC for the combined four biomarker signature was 0.92, as shown in FIG. 22.

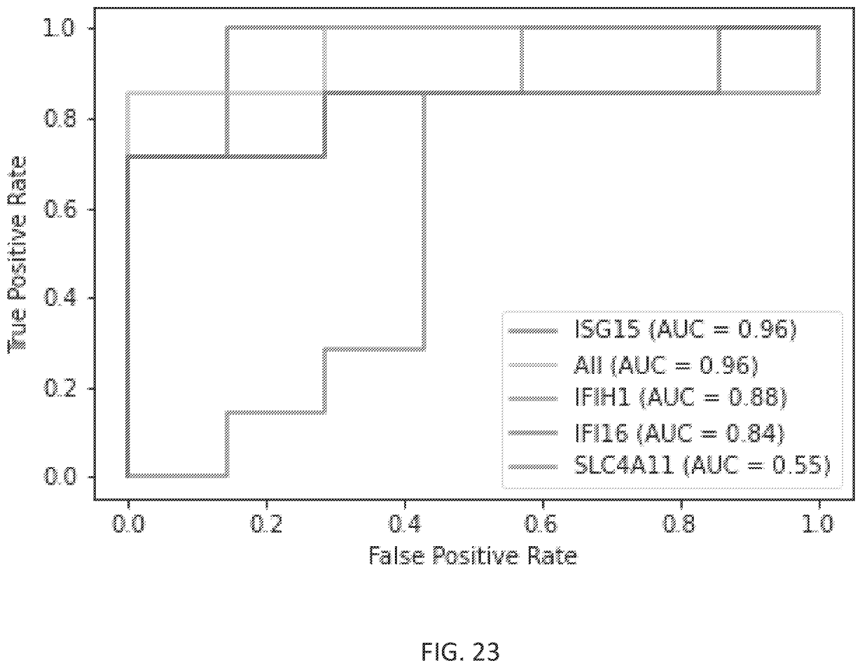

The genes ISG15, IFIH1, IFI16 and SLC4A11 were identified as having low association to saliva samples from subjects having SLE and a low association to saliva samples from subject having RA. FIG. 23 shows ROC curve analysis ISG15, IFIH1, IFI16 and SLC4A11, as well as all four genes together as a single biomarker signature. The AUC for the combined four biomarker signature was 0.96, as shown in FIG. 23.

Taken together, these results demonstrate that microvesicular RNA isolated from saliva samples can be used to differentiate between subjects having Sjögren's syndrome and subject that do not have Sjögren's syndrome. Moreover, these results demonstrate that the biomarker signatures described herein can be used to differentiate between subjects having Sjögren's syndrome and subject that do not have Sjögren's syndrome.

### Example 2—Cohort 2

Example 2 was performed according to the methods of Example 1, and methods described herein. The methods of Example 1 were replicated with an additional 53 subjects. Saliva samples were collected from 26 subjects with Sjögren's syndrome (15 subjects with SSA positive SS and 11 subjects with SSA negative SS) and 28 subjects who did not have Sjögren's syndrome (8 healthy subjects with no sicca symptoms and 20 healthy subjects with sicca symptoms). SSA positive samples were accepted as Sjögren's Syndrome in the medical community. SSA negative samples have been confirmed as Sjögren's Syndrome via lip biopsy. Samples were sequenced on Illumina NextSeq 2000 sequencers (paired-end sequencing, 76 bp read lengths) to an average of 106 million reads per sample (minimum read depth: 45,030,099 reads)

As in Example 1, Differential Expression analysis was performed on the sequencing data obtained from the SSA positive Sjögren's syndrome saliva samples (SSA+SS), SSA negative Sjögren's syndrome saliva samples (SSA-SS), saliva samples from all Sjögren's syndrome subjects (SSA+/−SS), saliva samples from heathy subjects without sicca symptoms (Healthy, Sicca−), Saliva samples from healthy subjects with sicca symptoms (Healthy, Sicca+), and saliva samples from all healthy subjects (Healthy, Sicca+/−) in cohort 2.

Table 6 shows the number of differentially expressed genes between the SSA positive Sjögren's syndrome samples compared to healthy (with and without sicca symptoms) and SSA negative Sjögren's syndrome samples; the number of upregulated genes in SSA positive Sjögren's syndrome samples; and the number of downregulated genes in SSA positive Sjögren's syndrome samples. Values in parentheses are the numbers of differentially expressed genes after a log fold shrink change filter.

TABLE 6
Differential Expression: SSA + Sjögren's syndrome

| | | Genes | Genes |
|---|---|---|---|
| | Differentially | Upregulated in | Downregulated |
| SSA + SS vs.: | Expressed Genes | SSA + SS | in SSA + SS |
| Healthy, Sicca− | 530 | 463 (264) | 67 (22) |
| Healthy, Sicca+ | 151 | 85 (63) | 66 (17) |
| SSA − SS | 73 | 47 (33) | 26 (2) |
| Healthy, Sicca+/− | 144 | 71 (57) | 73 (14) |

Table 7 shows the number of differentially expressed genes between the SSA negative Sjögren's syndrome samples compared to healthy (with and without sicca symptoms) and SSA positive Sjögren's syndrome samples; the number of upregulated genes in SSA negative Sjögren's syndrome samples; and the number of downregulated genes in SSA negative Sjögren's syndrome samples. Values in parentheses are the numbers of differentially expressed genes after a log fold shrink change filter.

TABLE 7
Differential Expression: SSA − Sjögren's syndrome

| | | Genes | Genes |
|---|---|---|---|
| | Differentially | Upregulated in | Downregulated in |
| SSA − SS vs.: | Expressed Genes | SSA − SS | SSA − SS |
| Healthy, Sicca− | 116 | 22 (7) | 94 (7) |
| Healthy, Sicca+ | 51 | 31 (15) | 20 (1) |
| SSA + SS | 73 | 26 (2) | 47 (33) |
| Healthy, Sicca+/− | 44 | 13 (1) | 31 (14) |

Table 8 shows the number of differentially expressed genes between all Sjögren's syndrome samples (SSA positive and SSA negative) compared to healthy (with and without sicca symptoms); the number of upregulated genes in all Sjögren's syndrome samples; and the number of downregulated genes in all Sjögren's syndrome samples. Values in parentheses are the numbers of differentially expressed genes after a log fold shrink change filter.

TABLE 8
Differential Expression: All Sjögren's Syndrome Samples

| | | Genes | Genes |
|---|---|---|---|
| All Sjögren's | Differentially | Upregulated in | Downregulated |
| Samples vs.: | Expressed Genes | SS | in SS |
| Healthy, Sicca+/− | 177 | 135 (74) | 42 (8) |
| Healthy, Sicca− | 155 | 109 (58) | 46 (18) |

Table 9 shows the number of differentially expressed genes between samples from healthy subjects without sicca symptoms compared to samples from healthy subjects with sicca symptoms; the number of upregulated genes in samples from healthy subjects with sicca symptoms; and the number of downregulated genes in samples from healthy subjects with sicca symptoms. Values in parentheses are the numbers of differentially expressed genes after a log fold shrink change filter.

### Table 9—Differential Expression: Healthy, Sicca-Vs Healthy, Sicca+

TABLE 9
Differential Expression: Healthy, Sicca− vs Healthy, Sicca+

| | | Genes | Genes |
|---|---|---|---|
| | Differentially | Upregulated | Downregulated |
| Healthy, | Expressed | in Healthy, | in Healthy, |
| Sicca− vs.: | Genes | Sicca+ | Sicca+ |
| Healthy, Sicca+ | 60 | 45 (5) | 15 (2) |

Gene signatures used to train logistic regression models using cohort 1 data in Example 1 were tested using SSA positive Sjögren's syndrome subjects and healthy subjects (with and without sicca symptoms) from cohort 2. Sample numbers 1-5, 7-12, 14-17 are samples collected from SSA positive Sjögren's syndrome subjects. Sample numbers 29-44, 46-52, 54-58 are samples collected from healthy subjects.

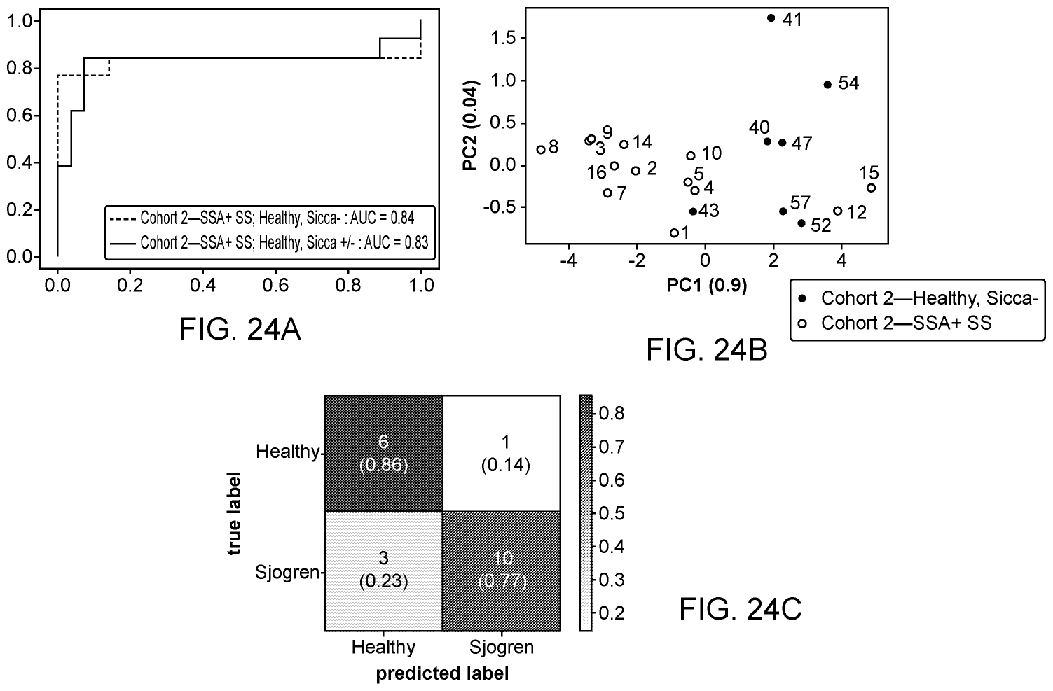

FIG. 24A shows receiver-operating characteristic (ROC) curve analysis for a biomarker signature comprising the biomarkers ISG15, RSAD2, TRIM38 and IFI6. The AUC for the biomarker signature was 0.84 for SSA positive SS samples compared to samples from healthy subjects without sicca symptoms. The AUC for the biomarker signature was 0.83 for SSA positive SS samples compared to samples from healthy subjects with and without sicca symptoms. FIG. 24B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 24C shows a Confusion Matrix summarizing the performance of the gene signature on the subjects from cohort 2.

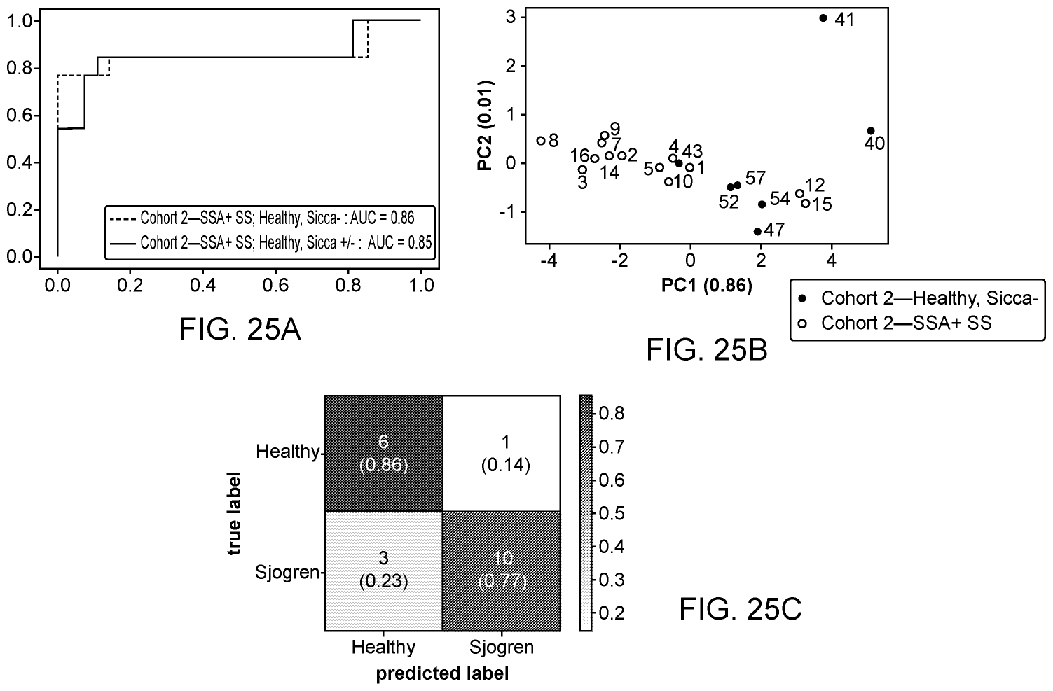

FIG. 25A shows ROC curve analysis for a biomarker signature comprising the biomarkers IFIH1, DDX60, OAS3 and ZC3HAV1. The AUC for the biomarker signature was 0.86 for SSA positive SS samples compared to samples from healthy subjects without sicca symptoms. The AUC for the biomarker signature was 0.85 for SSA positive SS samples compared to samples from healthy subjects with and without sicca symptoms. FIG. 25B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 25C shows a Confusion Matrix summarizing the performance of the gene signature on the subjects from cohort 2.

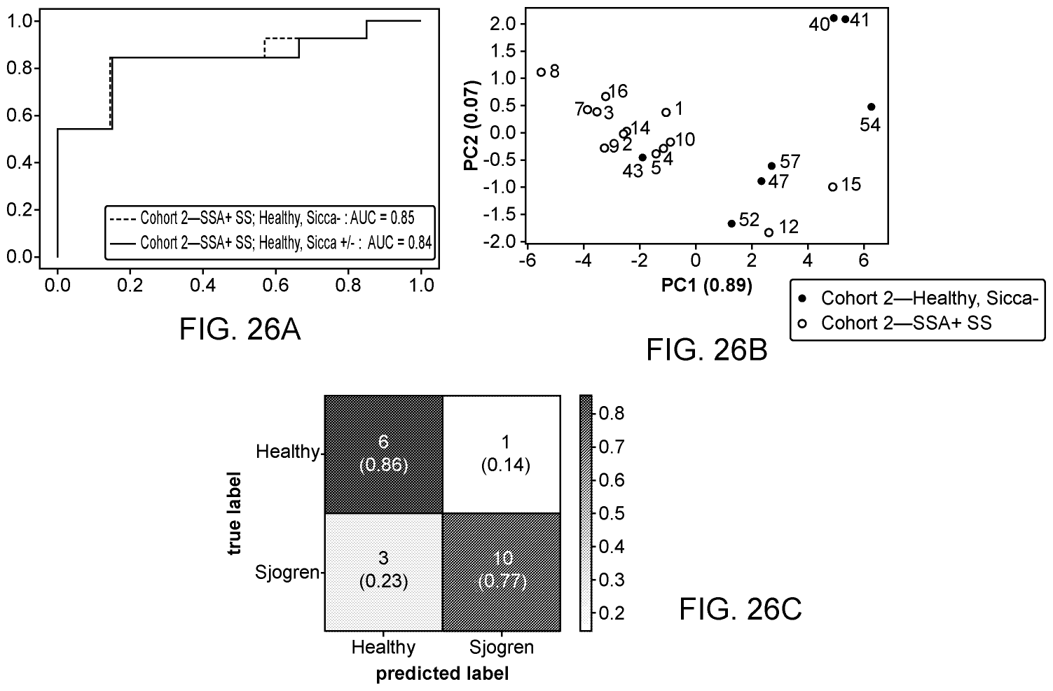

FIG. 26A shows ROC curve analysis for a biomarker signature comprising the biomarkers RSAD2, IFI6, IFIT5 and CMPK2. The AUC for the biomarker signature was 0.85 for SSA positive SS samples compared to samples from healthy subjects without sicca symptoms. The AUC for the biomarker signature was 0.84 for SSA positive SS samples compared to samples from healthy subjects with and without sicca symptoms. FIG. 26B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 26C shows a Confusion Matrix summarizing the performance of the gene signature on the subjects from cohort 2.

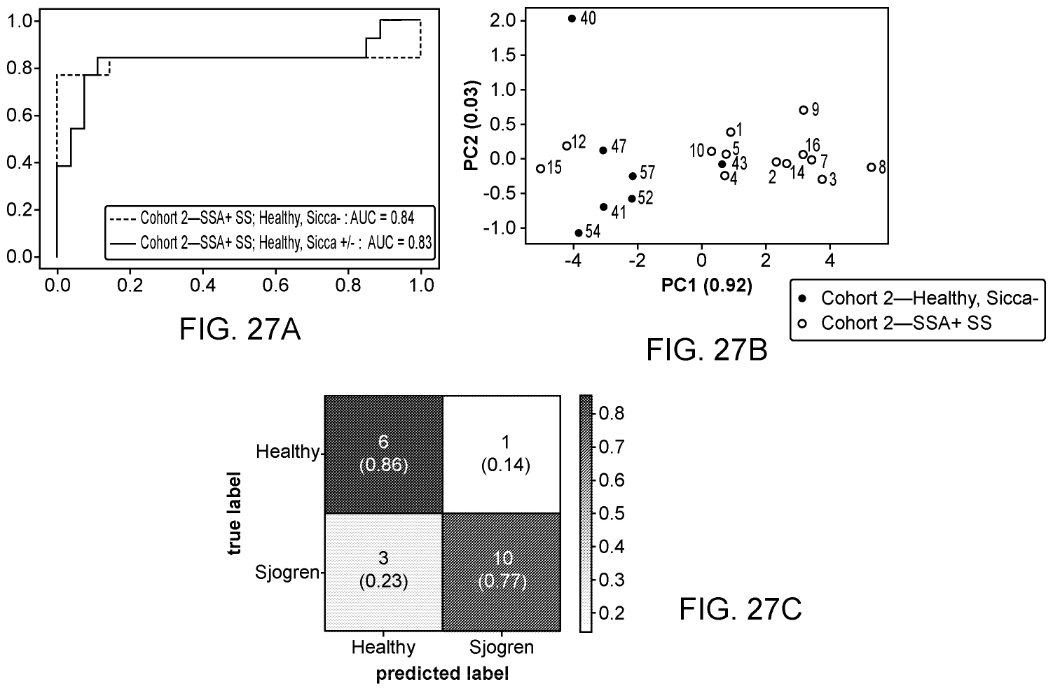

FIG. 27A shows ROC curve analysis for a biomarker signature comprising the biomarkers DDX60, OAS3, IFI6 and RSAD2. The AUC for the biomarker signature was 0.84 for SSA positive SS samples compared to samples from healthy subjects without sicca symptoms. The AUC for the biomarker signature was 0.83 for SSA positive SS samples compared to samples from healthy subjects with and without sicca symptoms. FIG. 27B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 27C shows a Confusion Matrix summarizing the performance of the gene signature on the subjects from cohort 2.

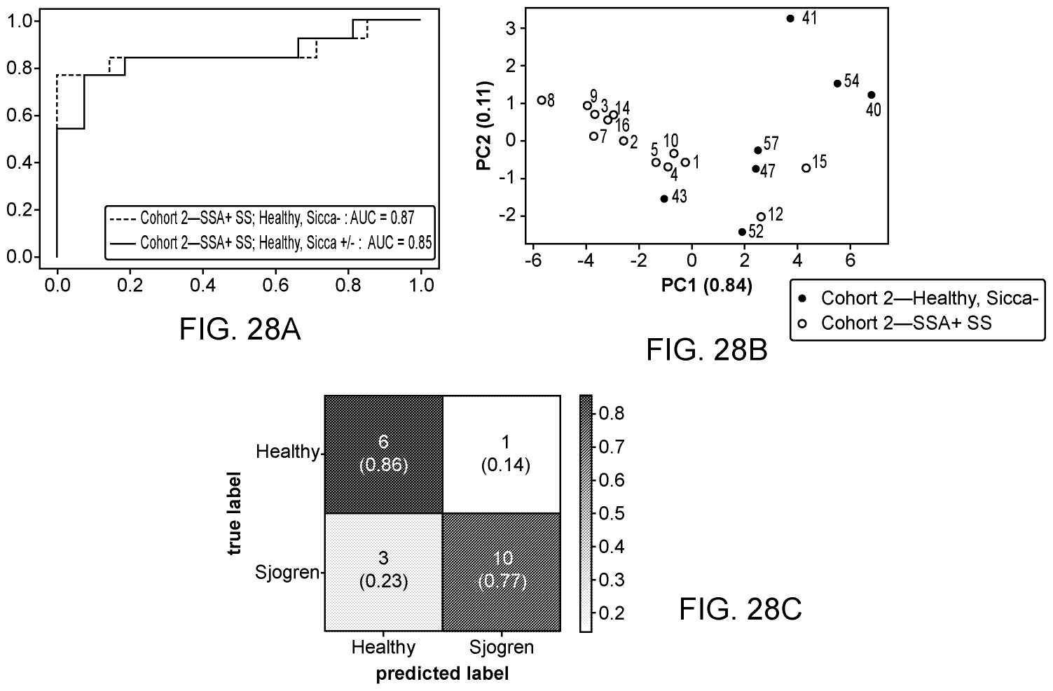

FIG. 28A shows ROC curve analysis for a biomarker signature comprising the biomarkers CMPK2, OAS1, OASL and ISG15. The AUC for the biomarker signature was 0.87 for SSA positive SS samples compared to healthy samples from subjects without sicca symptoms. The AUC for the biomarker signature was 0.85 for SSA positive SS samples compared to healthy samples from subjects with and without sicca symptoms. FIG. 28B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 28C shows a Confusion Matrix summarizing the performance of the gene signature on the subjects from cohort 2.

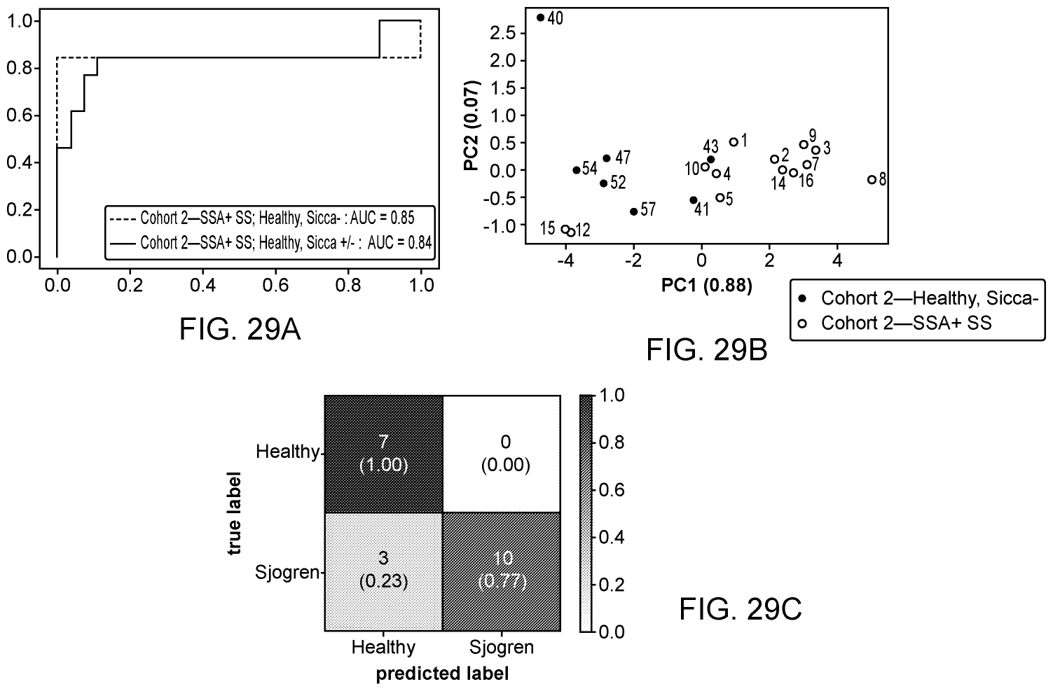

FIG. 29A shows ROC curve analysis for a biomarker signature comprising the biomarkers ISG15, RSAD2, IFI16 and OAS1. The AUC for the biomarker signature was 0.85 for SSA positive SS samples compared to samples from healthy subjects without sicca symptoms. The AUC for the biomarker signature was 0.84 for SSA positive SS samples compared to healthy samples from subjects with and without sicca symptoms. FIG. 29B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 29C shows a Confusion Matrix summarizing the performance of the gene signature on the subjects from cohort 2.

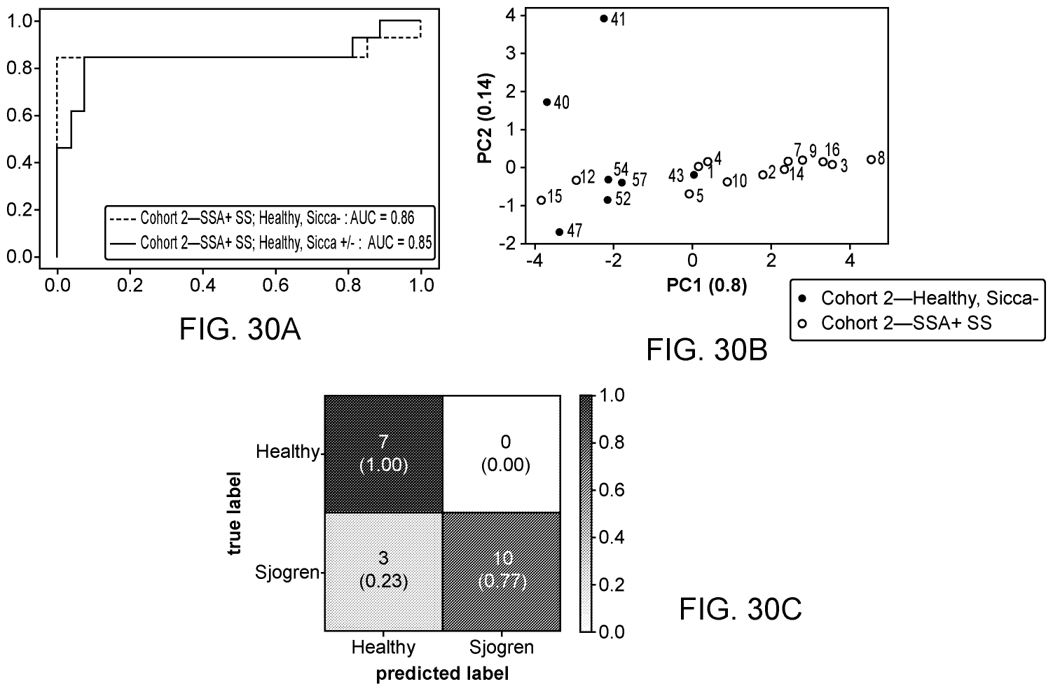

FIG. 30A shows ROC curve analysis for a biomarker signature comprising the biomarkers ISG15, IFIH1, EPSTI1 and IFI16. The AUC for the biomarker signature was 0.86 for SSA positive SS samples compared to samples from healthy subjects without sicca symptoms. The AUC for the biomarker signature was 0.85 for SSA positive SS samples compared to samples from healthy subjects with and without sicca symptoms. FIG. 30B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 30C shows a Confusion Matrix summarizing the performance of the gene signature on the subjects from cohort 2.

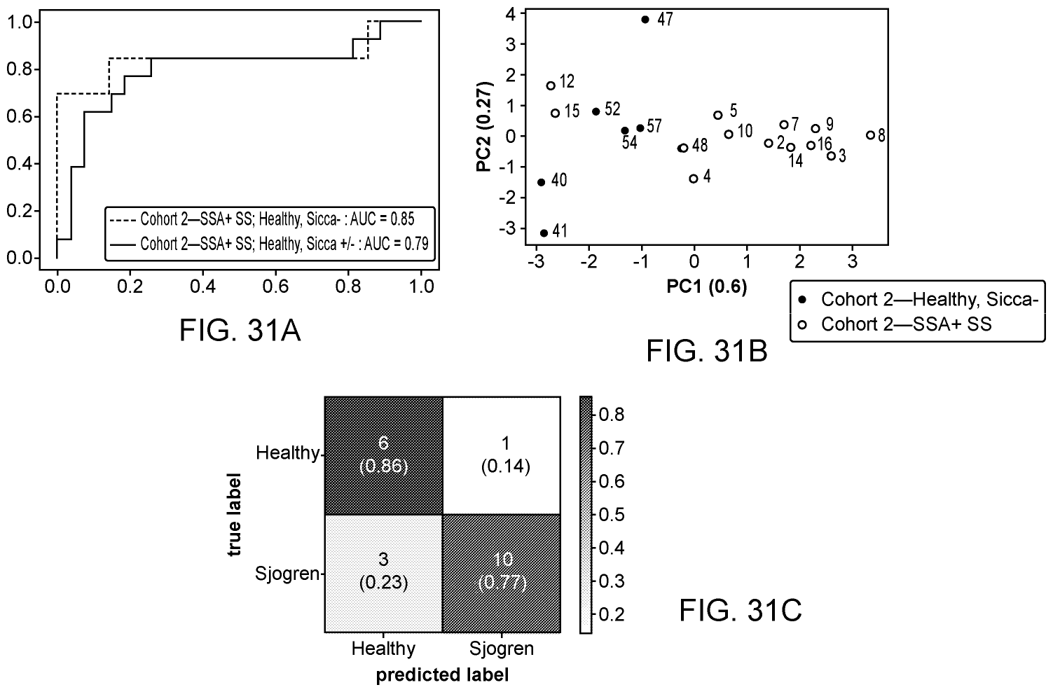

FIG. 31A shows ROC curve analysis for a biomarker signature comprising the biomarkers ISG15, IFIH1, IFI16 and SLC4A11. The AUC for the biomarker signature was 0.85 for SSA positive SS samples compared to samples from healthy subjects without sicca symptoms. The AUC for the biomarker signature was 0.79 for SSA positive SS samples compared to healthy samples from subjects with and without sicca symptoms. FIG. 31B shows a Principal Component Analysis (PCA) of the expression of the gene signature in sequencing analysis of microvesicular RNA isolated from salivary microvesicles from subjects with Sjögren's syndrome and healthy subjects in cohort 2. FIG. 31C shows a Confusion Matrix summarizing the performance of the gene signature on the subjects from cohort 2.

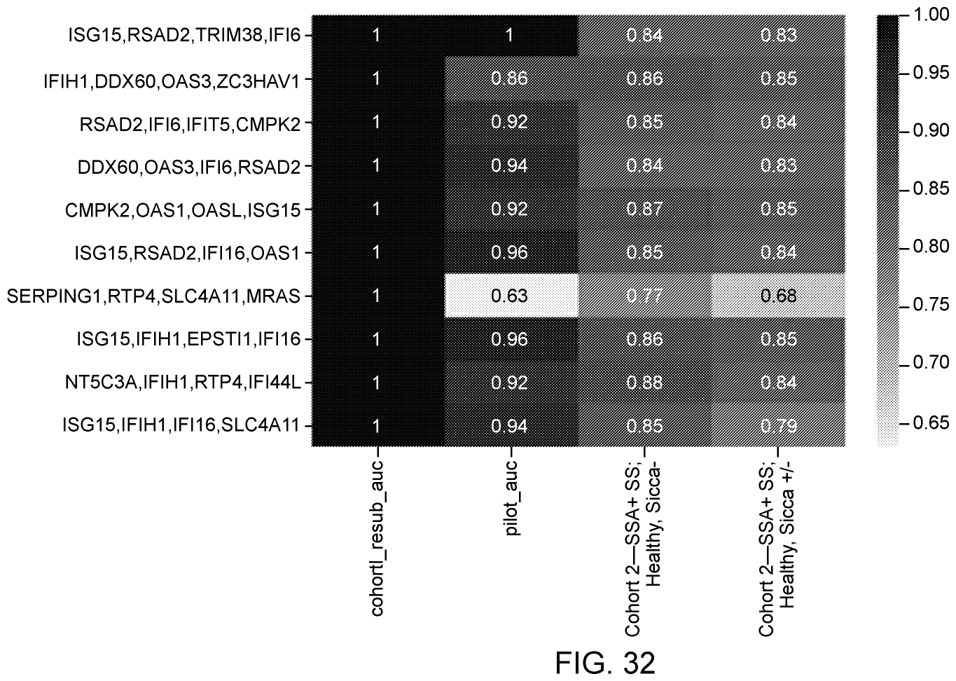

When the biomarker signatures developed using cohort 1 data in Example 1 were tested using the data in cohort 2, most biomarker sets achieved an AUC of above 0.8. FIG. 32 shows a heatmap summarizing the AUCs for 10 gene signatures developed using cohort 1 data from Example 1, tested using a pilot cohort and cohort 2 samples. Without wishing to be bound by theory, these results demonstrate that the biomarker signatures and models developed herein have the potential to be generalized. Taken together, these results support the conclusion that microvesicular RNA isolated from saliva samples can be used to differentiate between subjects having Sjögren's syndrome and subject that do not have Sjögren's syndrome, regardless of presentation of sicca symptoms.

In each of the above cases, some samples in cohort 2 do not cluster with their respective groups. A similar set of samples does not cluster appropriately in cohort 2. Specifically, two samples were not able to be classified correctly with any of the gene signatures developed using cohort 1 data in Example 1.

### Example 3—Cohort 2

Example 3 was preformed according to the methods of Example 2, and methods described herein. Sequencing data from Example 2/cohort 2 was used to preform Differential Expression analysis. Table 10 shows the number of differentially expressed exosome genes between the SSA positive Sjögren's syndrome samples compared to healthy (with and without sicca symptoms) and SSA negative Sjögren's syndrome samples; SSA negative Sjögren's syndrome samples compared to healthy (with and without sicca symptoms); and healthy samples with sicca symptoms compared to healthy samples without sicca symptoms.

Differential expression was defined as having an absolute value of log 2 fold change of >0.05 after shrinkage and an adjusted P-value of <0.05. Feature selection using Boruta was performed on the differentially expressed genes. Boruta confirmed genes were entered into model fitting where Recursive feature elimination (RFE) was used to select the genes with distinguishing power. Genes confirmed by RFE were included in a model where leave-one-out cross validation (LOOCV) was applied to select the best number and best features to be included in the final model (maximum set to four features to avoid overfitting). All models used herein were logistic regression.

TABLE 10
Differential Expression

| | | | | Leave |
|---|---|---|---|---|
| | | | | one-out |
| | Differentially | | Gene signature | cross |
| | Expressed | Boruta | included in the | validation |
| | Genes | confirmed | model | AUC |
| SSA + SS vs Healthy, | 243 | 26 | C19orf48, RNF26, NOS2 | 0.97 |
| Sicca − (n = 23) | | | | |
| SSA + SS vs Healthy, | 66 | 26 | ZNF775, ANKRD29, OAS1 | 0.91 |
| Sicca + (n = 35) | | | | |
| SSA − SS vs Healthy, | 9 | 9 | ARSL, LAMB1, TUBB3 | 0.93 |
| Sicca − (n = 19) | | | | |
| SSA − SS vs Healthy, | 11 | 2 | ARSL, CTSC | 0.80 |
| Sicca + (n = 31) | | | | |
| SSA + SS vs SSA − SS | 32 | 16 | ZCCHC4, UGT2A1, IFIT1, | 0.97 |
| (n = 26) | | | CD101-AS1 | |
| Healthy, Sicca − vs | 6 | 0 | | — |
| Healthy, Sicca + (n = 28) | | | | |

After model fitting, 243 genes were identified as differentially expressed between SSA positive Sjögren's syndrome samples and samples from healthy subjects without sicca symptoms. Of those 243 genes, 26 were Boruta confirmed and C19orf48, RNF26, and NOS2 were included in the model. Using LOOCV, AUC for the biomarker signature was 0.97.

After model fitting, 66 genes were identified as differentially expressed between SSA positive Sjögren's syndrome samples and samples from healthy subjects with sicca symptoms. Of those 66 genes, 26 were Boruta confirmed and ZNF775, ANKRD29, and OAS1 were included in the model. Using LOOCV, AUC for the biomarker signature was 0.91.

After model fitting, 9 genes were identified as differentially expressed between SSA negative Sjögren's syndrome samples and samples from healthy subjects without sicca symptoms. Of those 9 genes, 9 were Boruta confirmed and ARSL, LAMB1, and TUBB3 were included in the model. Using LOOCV, AUC for the biomarker signature was 0.93.

After model fitting, 11 genes were identified as differentially expressed between SSA negative Sjögren's syndrome samples and samples from healthy subjects with sicca symptoms. Of those 11 genes, 2 were Boruta confirmed and ARSL and CTSC were included in the model. Using LOOCV, AUC for the biomarker signature was 0.80.

After model fitting, 32 genes were identified as differentially expressed between SSA negative Sjögren's syndrome samples and SSA positive Sjögren's syndrome samples. Of those 32 genes, 16 were Boruta confirmed and ZCCHC4, UGT2A1, IFIT1, and CD101-AS1 were included in the model. Using LOOCV, AUC for the biomarker signature was 0.97.

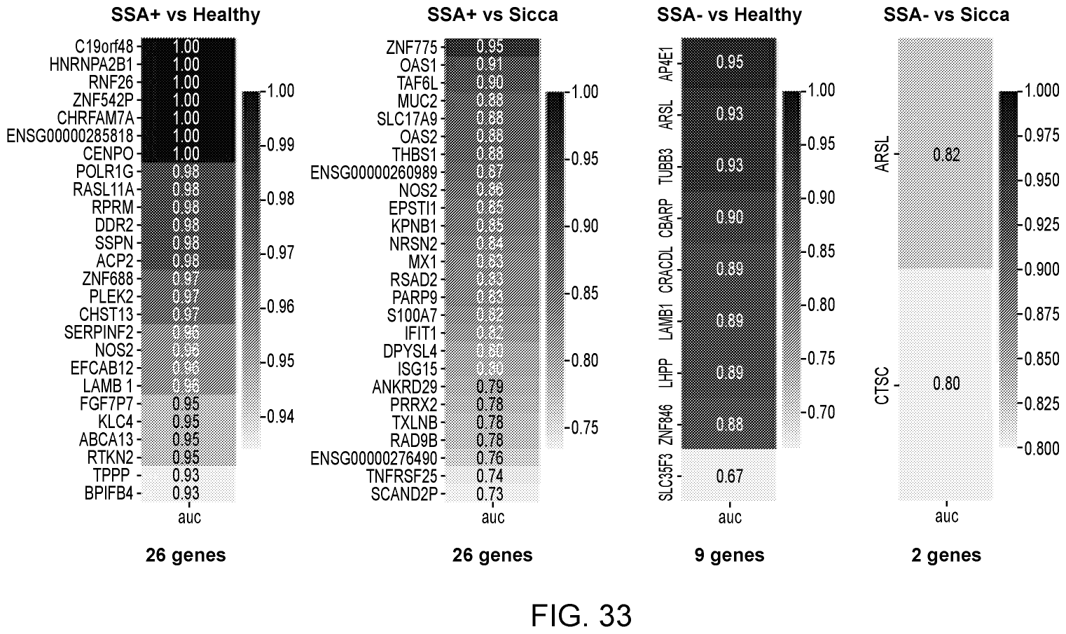

FIG. 33 is a heatmap summarizing the univariate analysis of all Boruta selected genes in the pairwise comparisons outlined in Table 10.

### SSA Positive Sjögren's Syndrome Gene Signature:

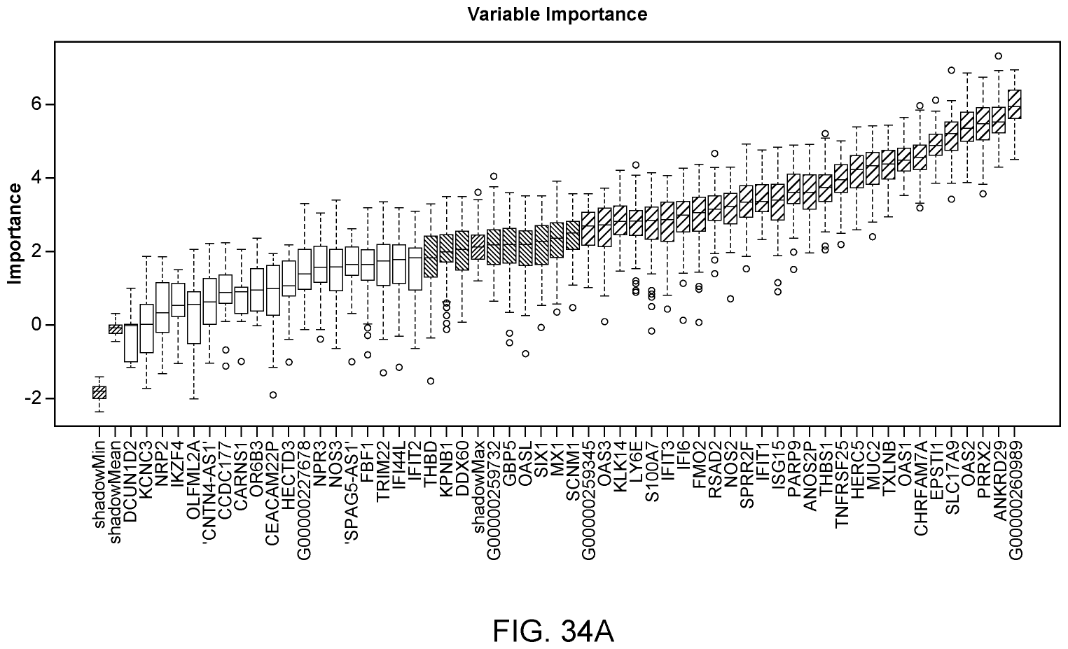

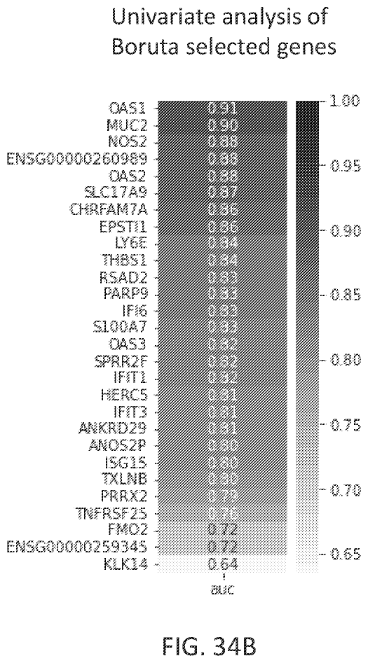

FIG. 34A shows the variable importance for various genes identified by the feature selection (RFE). FIG. 34B shows a heatmap summarizing univariate analysis of 28 Boruta selected genes. The 28 genes were selected from 56 genes that were differentially expressed between healthy control saliva samples (with and without sicca symptoms) and SSA positive Sjögren's syndrome saliva samples (n=40). 7 out of 28 Boruta genes overlap with those identified in Example 1 from cohort 1.

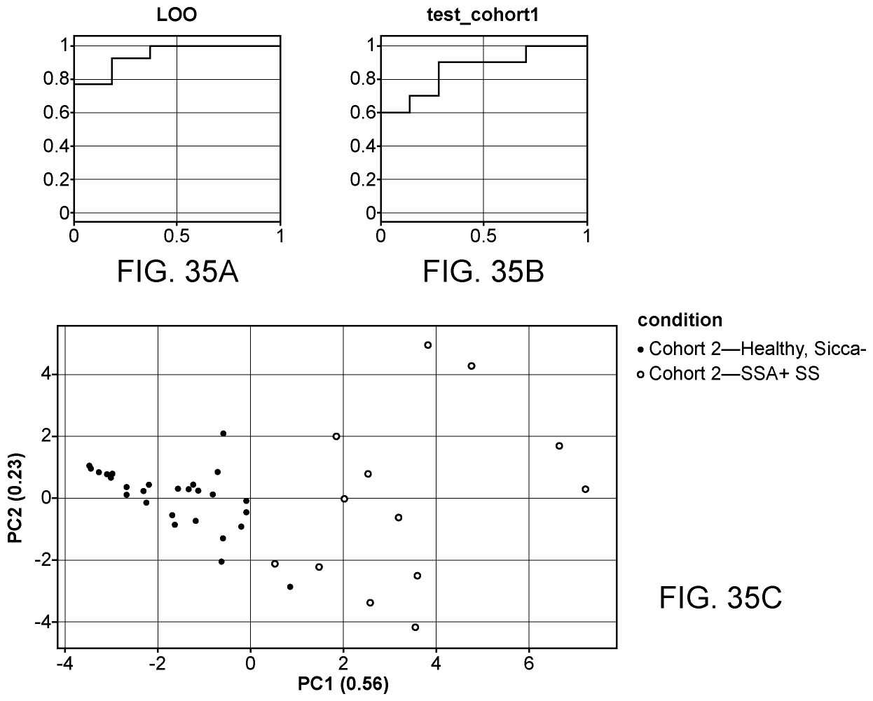

FIG. 35A shows an ROC curve analysis for a biomarker signature comprising the biomarkers ANKRD29, PRRX2, OAS1, and MUC2. This gene signature developed by training on cohort 2 data was tested using samples from cohort 1. FIG. 35B shows an ROC curve analysis for the biomarker signature for use in identifying the presence SSA positive Sjögren's syndrome in a subject based on the expression of the genes in microvesicular RNA extracted from a saliva sample from the subject from cohort 1. FIG. 35C shows a Principal Component Analysis (PCA) of the expression of the gene signature in sequencing analysis of microvesicular RNA isolated from salivary microvesicles from subjects with SSA positive Sjögren's syndrome and healthy (with and without sicca symptoms) subjects in cohort 2. These results demonstrate a clear separation between healthy samples and SS samples with only 1 sample not clearly separated.

Taken together, these results demonstrate that microvesicular RNA isolated from saliva samples can be used to differentiate between subjects having SSA positive Sjögren's syndrome and subject that do not have Sjögren's syndrome that the biomarker signatures described herein can be used to differentiate between subjects having SSA positive Sjögren's syndrome and subject that do not have Sjögren's syndrome.

### SSA Negative Sjögren's Syndrome Gene Signature:

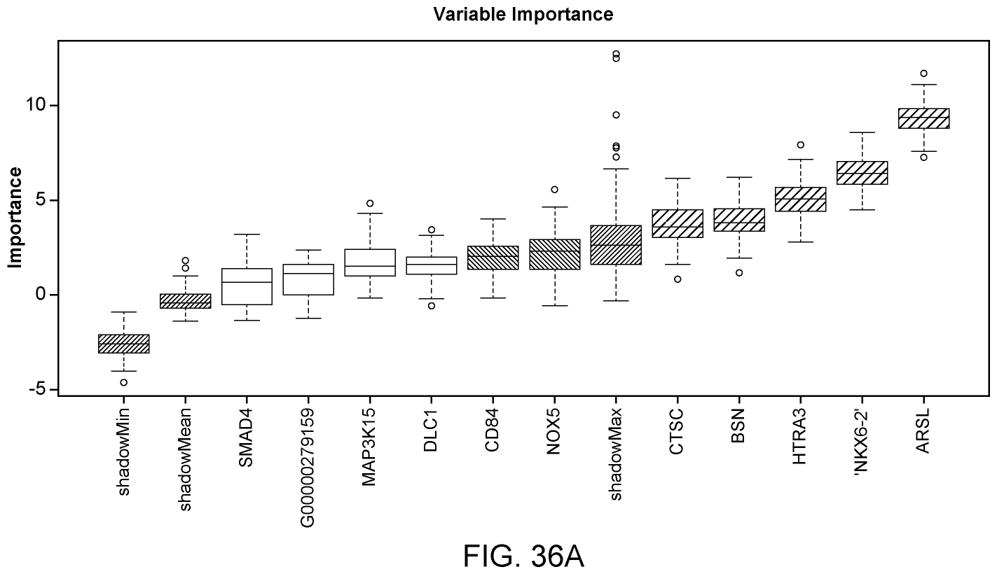

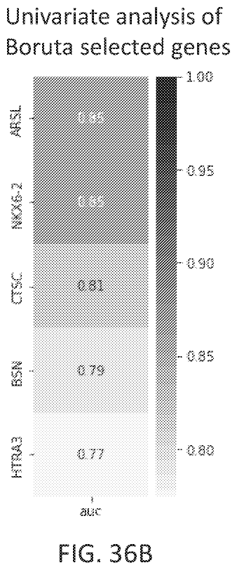

FIG. 36A shows the variable importance for various genes identified by the feature selection (RFE). FIG. 36B shows a heatmap summarizing univariate analysis of 5 Boruta selected genes. The 5 genes were selected from 11 genes that were differentially expressed between healthy control saliva samples (with and without sicca symptoms) and SSA negative Sjögren's syndrome saliva samples (n=38).

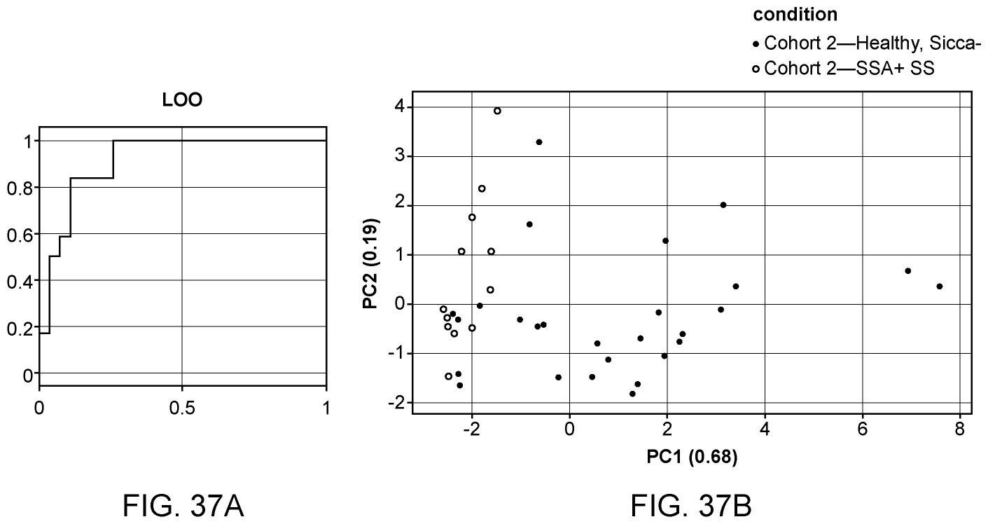

FIG. 37A shows an ROC curve analysis for a biomarker signature comprising the biomarkers ARSL, NKX6-2, HTRA3, and BSN. FIG. 37B shows a Principal Component Analysis (PCA) of the expression of the gene signature in sequencing analysis of microvesicular RNA isolated from salivary microvesicles from subjects with SSA negative Sjögren's syndrome and healthy subjects (with and without sicca symptoms) in cohort 2. These results demonstrate that Healthy samples are mostly able to be separated from SS samples. Those samples that were not clearly separated did not belong to a specific subgroup of healthy samples (i.e., those with sicca symptoms or those without sicca symptoms).

Taken together, these results demonstrate that microvesicular RNA isolated from saliva samples can be used to differentiate between subjects having SSA negative Sjögren's syndrome and subjects that do not have Sjögren's syndrome and that the biomarker signatures described herein can be used to differentiate between subjects having SSA negative Sjögren's syndrome and subjects that do not have Sjögren's syndrome.

### SSA Negative Sjögren's Syndrome Vs SSA Positive Sjögren's Syndrome Gene Signature:

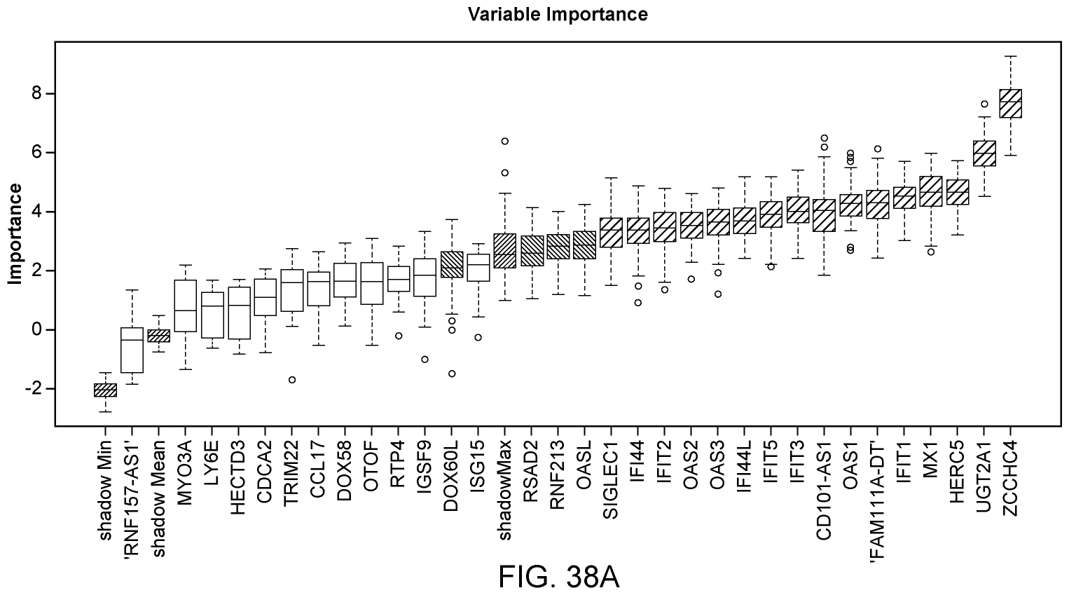

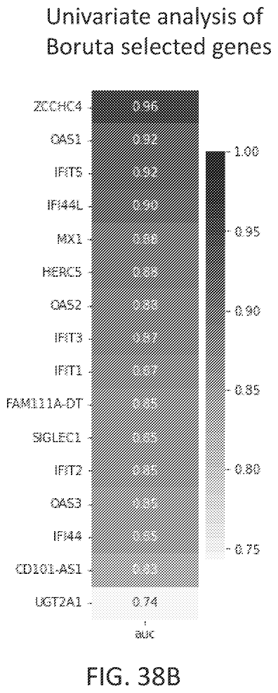

FIG. 38A shows the variable importance for various genes identified by the feature selection. FIG. 38B shows a heatmap summarizing univariate analysis of 16 Boruta selected genes. The 16 genes were selected from 32 genes that were differentially expressed between SSA positive Sjögren's syndrome saliva samples and SSA negative Sjögren's syndrome saliva samples (n=26).

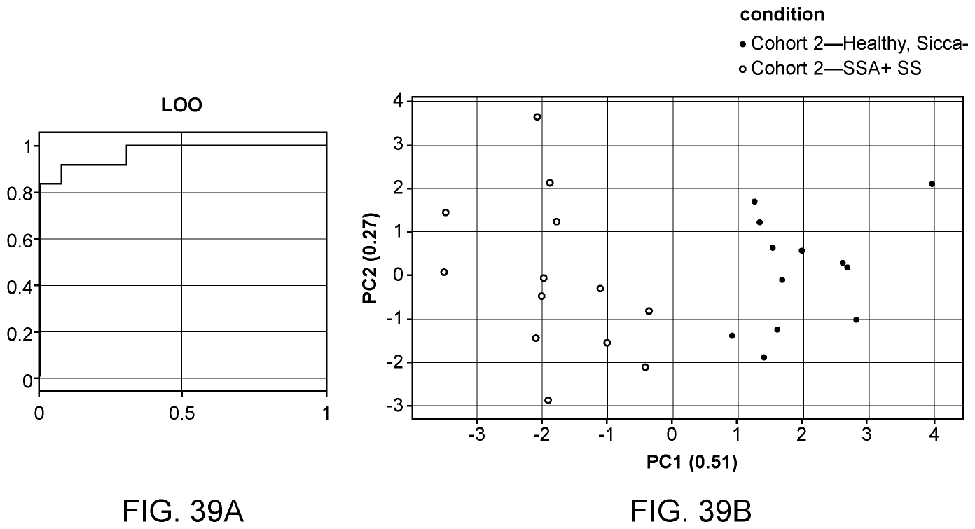

FIG. 39A shows an ROC curve analysis for a biomarker signature comprising the biomarkers ZCCHC4, UGT2A1, IFIT1, CD101-AS1. FIG. 39B shows a Principal Component Analysis (PCA) of the expression of the gene signature in sequencing analysis of microvesicular RNA isolated from salivary microvesicles from subjects with SSA negative Sjögren's syndrome and from salivary microvesicles from subjects with SSA positive Sjögren's syndrome in cohort 2. SSA negative SS was confirmed by lip biopsy. These results demonstrate a clear separation between SSA negative SS samples (confirmed by Lip biopsy) and SSA positive SS samples. Without wishing to be bound by theory, these data support the conclusion that SSA positive SS and SSA-SS are highly distinct populations.

Taken together, these results demonstrate that microvesicular RNA isolated from saliva samples can be used to differentiate between subjects having SSA positive Sjögren's syndrome and subjects having SSA negative Sjögren's syndrome and that the biomarker signatures described herein can be used to differentiate between subjects having SSA positive Sjögren's syndrome and subjects having SSA negative Sjögren's syndrome.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A method of determining if a subject is Sjögren's syndrome positive or is Sjögren's syndrome negative, the method comprising:
a) determining the expression level of at least one biomarker selected from at least one biomarker signature in microvesicular RNA isolated from a saliva sample from the subject;
b) inputting the expression levels from step (a) into an algorithm to generate a score; and
c) identifying if the subject is Sjögren's syndrome positive or is Sjögren's syndrome negative syndrome based on the score.

2. A method of identifying the risk of Sjögren's syndrome in a subject, the method comprising:
a) determining the expression level of at least one biomarker selected from at least one biomarker signature in microvesicular RNA isolated from a saliva sample from the subject;
b) inputting the expression levels from step (a) into an algorithm to generate a score; and
c) identifying the risk of Sjögren's syndrome based on the score.

3. A method of treating Sjögren's syndrome in a subject, the method comprising: a) determining the expression level of at least one biomarker selected from at least one biomarker signature in microvesicular RNA isolated from a saliva sample from the subject;
b) inputting the expression levels from step (a) into an algorithm to generate a score; c) administering at least one treatment to the subject based on the score.

4. A method of distinguishing between SSA positive Sjögren's syndrome and SSA negative Sjögren's syndrome in a subject, the method comprising:
a) determining the expression level of at least one biomarker selected from at least one biomarker signature in microvesicular RNA isolated from a saliva sample from the subject;
b) inputting the expression levels from step (a) into an algorithm to generate a score;
c) distinguishing between SSA positive Sjögren's syndrome and SSA negative Sjögren's syndrome in the subject based on the score.

5. The method of any one of the preceding claims, wherein the at least one biomarker signature is selected from:
i) ISG15, RSAD2, TRIM38, and IFI6;
ii) IFIH1, DDX60, OAS3 and ZC3HAV1;
iii) RSAD2, IFI6, IFIT5 and CMPK2;
iv) DDX60, OAS3, IFI6 and RSAD2;
v) CMPK2, OAS1, OASL and ISG15;
vi) ISG15, IFI16, RSAD2 and OAS1;
vii) IFIH1, ISG15, EPSTI1 and IFI16;
viii) SERPING1, RTP4, SLC4A11 and MRAS;
ix) NT5C3A, IFIH1, RTP4 and IFI44L; and
x) ISG15, IFIH1, IFI16 and SLC4A11.

6. The method of any one of claims 1-4, wherein the at least one biomarker signature is selected from:
i) ANKRD29, PRRX2, OAS1, and MUC2;
ii) ARSL, NKX6-2, HTRA3, and BSN; and
iii) ZCCHC4, UGT2A1, IFIT1, and CD101-AS1.

7. The method of any one of the preceding claims, wherein, step (a) comprises determining the expression level of at least two, or at least three of the biomarkers in the at least one biomarker signature.

8. The method of any one of the preceding claims, wherein, step (a) comprises determining the expression level of each of the biomarkers in the at least one biomarker signature.

9. The method of any one of the preceding claims, wherein the algorithm is the product of a feature selection wrapper algorithm, a machine learning algorithm, a trained classifier built from at least one predictive classification algorithm or any combination thereof.

10. The method of claim 9, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

11. The method of any one of the preceding claims, wherein the algorithm is the product of a feature selection wrapper algorithm, machine learning algorithm, trained classifier, logistic regression model or any combination thereof, that was trained to identify Sjögren's syndrome in a subject using:
a) the expression levels of the at least one biomarker selected from the at least one biomarker signature in at least one biological sample from at least one subject who does not have Sjögren's syndrome;
b) the expression levels of the at least one biomarker selected from the at least one biomarker signature in at least one biological sample from at least one subject who has Sjögren's syndrome;
c) the expression levels of the at least one biomarker selected from the at least one biomarker signature in at least one biological sample from at least one subject who has SSA positive Sjögren's syndrome;
d) the expression levels of the at least one biomarker selected from the at least one biomarker signature in at least one biological sample from at least one subject who has SSA negative Sjögren's syndrome;
e) the expression levels of the at least one biomarker selected from the at least one biomarker signature in at least one biological sample from a subject who does not have Sjögren's syndrome and who does not exhibit sicca symptoms;
f) the expression levels of the at least one biomarker selected from the at least one biomarker signature in at least one biological sample from a subject who does not have Sjögren's syndrome but exhibits sicca symptoms;
g) the expression levels of the at least one biomarker selected from the at least one biomarker signature in at least one biological sample from a subject who has at least on alternative disease/disorder, or
h) any combination thereof.

12. The method of any one of the preceding claims, wherein the saliva sample is collected using sample home-collection device.

13. The method of any one of the preceding claims, further comprising prior to step (a):
i) isolating a plurality of microvesicles from the saliva sample from the subject; and
ii) extracting microvesicular RNA from the plurality of isolated microvesicles.

14. The method of claim 13, further comprising at least one of:
i) prior to step (i), adding at least one stabilizing agent to the saliva sample, preferably wherein the at least one stabilizing agent is an RNAse inhibitor;
ii) filtering the saliva samples, preferably filtering comprises using a filter with an average pore size of about 0.8 μm;
iii) fragmenting the extracted microvesicular RNA;
iv) contacting the extracted microvesicular RNA with Solid-phase reversible immobilization (SPRI) beads; and
v) amplifying the extracted microvesicular RNA is using PCR, preferably wherein the amplification is performed for about 18 cycles.

15. The method of any one of the preceding claims, wherein the plurality of microvesicles is isolated from the saliva sample by contacting the saliva sample with at least one affinity agent that binds to at least one surface marker present on the surface the at least one microvesicle.

16. The method of any one of the preceding claims, wherein step (a) further comprises:
(i) determining the expression level of at least one reference biomarker;
(ii) normalizing the expression level of the at least one biomarker to the expression level of the at least one reference biomarker.

17. The method of any one of the preceding claims, wherein inputting the expression levels from step (a) into an algorithm to generate a score comprises inputting the normalized expression levels from step (a) into an algorithm to generate a score.

18. The method of any one of the preceding claims, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

19. The method of claim 18, wherein determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

20. The method of any one of the preceding claims, wherein the method
i) has a negative predictive value of at least about 80%, or at least about 85%, or at least about 90%, or at least about 95%, or at least about 99%, or at least about 99.9%;
ii) has a positive predictive value of at least about 80%, or at least about 85%, or at least about 90%, or at least about 95%, or at least about 99%, or at least about 99.9%;
iii) has a sensitivity of at least about 80%, or at least about 85%, or at least about 90%, or at least about 95%, or at least about 99%, or at least about 99.9%;
iv) has a specificity of at least about 80%, or at least about 85%, or at least about 90%, or at least about 95%, or at least about 99%, or at least about 99.9%; or
v) any combination thereof.

21. The method of any one of the preceding claims, wherein measuring expression levels in step (a) further comprises selectively enriching for the at least one biomarker.

22. The method of any one of the preceding claims, wherein the at least one biomarker is selectively enriched by hybrid-capture, preferably wherein:
i) the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome; and/or
ii) the hybrid-capture results in a significant depletion in microbial nucleic acids

23. The method of any one of the preceding claims, further comprising administering at least one treatment to a subject identified as having Sjögren's syndrome.

24. The method of any one of the preceding claims, wherein the at least one treatment comprises:
i) administering at least one therapeutically effective amount of cevimeline, pilocarpine, a supersaturated calcium phosphate rinse, cyclosporine, tacrolimus eye drops, abatacept, rituximab, tocilizumab, hydroxypropyl cellulose, lifitegrast, LO2A eye drops, rebamipide eye drops, topical autologous serum, intravenous immunoglobulins, dexamethasone eye drops, an immunosuppressive medication, a nonsteroidal anti-inflammatory medication, an arthritis medication, an antifungal medication, hydroxychloroquine, methotrexate, LOU064, INCB050465 or any combination thereof;
ii) surgery, preferably wherein the surgery comprises sealing the tear ducts of the subject;
iii) administering at least one therapeutically effective amount of UCB5857, CFZ533, AMG557, IL-2, a combination of rituximab and belimumab, tocilizumab, abatacept, RSLV-132, VIB4920, iscalimab, baricitinib, nipocalimab, dazodalibep, MHV370, S95011, efgartigimod, tofacitinib, iguratomid, anifrolumab, branebrutinib, telitacicept, SAR441344, or any combination thereof;
iv) at least one AAV-based therapy, preferably wherein the at least one AAV-based therapy comprises an AAV-based vector comprising a nucleic acid sequence encoding at least one aquaporin protein, or a functional fragment thereof; or
iv) any combination thereof.
